(19) United States
(12) Patent Application Publication
(10) Pub. No.: US 2011/0030103 A1
(43) Pub. Date: Feb. 3, 2011

(54) LIPOPEPTIDES AND LIPOPEPTIDE SYNTHETASES

(76) Inventors: Gabriel Reznik, Brookline, MA (US); Kevin A. Jarrell, Lincoln, MA (US); Prashanth Vishwanath, Brighton, MA (US)

Correspondence Address: CHOATE, HALL & STEWART LLP, TWO INTERNATIONAL PLACE, BOSTON, MA, 02110, US

(21) Appl. No.: 12/596,276

(22) PCT Filed: Apr. 16, 2008
(86) PCT No.: PCT/US08/60497

§ 371 Date: Jul. 6, 2010

Related U.S. Application Data
(60) Provisional application No. 60/923,679, filed on Apr. 16, 2007.
(60) Provisional application No. 60/945,527, filed on Jun. 21, 2007.
(60) Provisional application No. 61/026,610, filed on Feb. 6, 2008.

(51) Int. Cl. C12N 9/00; A01H 5/00; C07K 7/06

(52) U.S. Cl. 800/298; 435/183; 530/329; 530/321

(57) ABSTRACT

Novel lipopeptides, and engineered polypeptides useful in synthesizing lipopeptides are provided. Also provided are methods of making lipopeptides using engineered polypeptides, and methods of using lipopeptides, e.g., as insecticidal and/or antimicrobial agents.

### CROSS REFERENCE TO RELATED APPLICATIONS

This application is copending with, shares at least one common inventor with, and claims priority to U.S. provisional patent application No. 60/923,679, filed Apr. 16, 2007. This application is also copending with, shares at least one common inventor with, and claims priority to U.S. provisional patent application No. 60/945,527, filed Jun. 21, 2007. This application is also copending with, shares at least one common inventor with, and claims priority to U.S. provisional patent application No. 61/026,610, filed Feb. 6, 2008. The entire contents of each of these applications are hereby incorporated by reference.

### BACKGROUND

Nonribosomal peptide synthetases are large multienzyme complexes that produce bioactive peptide compounds. Peptides assembled by nonribosomal synthetases incorporate common amino acids as well as uncommon and modified amino acids, including D-amino acids, acylated amino acids, methylated residues, formylated residues, and glycosylated residues. Lipopeptides, which include an amino acid modified by a fatty acid, are commercially important compounds in that many possess surfactant, antimicrobial, and insecticidal properties. The fatty acid moiety is a structural feature that confers useful characteristics, such as the ability to bind hydrophobic targets (e.g., cell membranes).

Lipopeptides with surfactant, antibiotic, and/or insecticidal properties are produced naturally in microorganisms. For example, Bacillus species produce numerous different types of lipopeptides including surfactin, fengycin, and plipastatin. Surfactin is a cyclic heptapeptide with both antimicrobial and potent surfactant activities. Fengycin is an antifungal cyclic decapeptide. Plipastatins, originally isolated as inhibitors of porcine pancreatic phospholipase A2, are decapeptides with fungicidal activity to plant pathogens such as Botrytis, Pyricularia and Alternaria. Many other microbial species produce lipopeptides with beneficial properties. Daptomycin is a thirteen amino acid lipopeptide produced by Streptomyces roseosporus, which has bactericidal activity against drug-resistant enterococcal, Staphylococcal, and Streptococcal species. Daptomycin (Cubicin®) is approved for treatment of methicillin-resistant and methicillin-susceptible Staphylococcus aureus infections in humans.

### SUMMARY OF THE INVENTION

In certain embodiments, the present invention provides compositions and methods useful in the generation of lipopeptides. In certain embodiments, the present invention provides an engineered lipopeptide synthetase polypeptide. For example, in some embodiments, the present invention provides an engineered lipopeptide synthetase polypeptide which is a deletion mutant of a naturally occurring lipopeptide synthetase polypeptide, and which produces a lipopeptide having a different number of amino acids (e.g., one, two, three, or four amino acids fewer) than the lipopeptide produced by the corresponding naturally ocurring lipopeptide synthetase polypeptide. In certain embodiments, an engineered lipopeptide synthetase polypeptide is a deletion mutant of a naturally occurring lipopeptide synthetase polypeptide that comprises a first and second peptide synthetase domain, wherein each peptide synthetase domain comprises a condensation (C) domain, an adenylation (A) domain, and a thiolation (T) domain, and wherein the engineered polypeptide comprises a deletion of at least a portion of a C domain, a portion of an A domain, or a portion of a T domain relative to the corresponding naturally occurring polypeptide.

In certain embodiments, the present invention provides an engineered lipopeptide synthetase polypeptide comprising a first peptide synthetase domain of a first peptide synthetase, and a second peptide synthetase domain of a second peptide synthetase.

In certain embodiments, the present invention provides an engineered lipopeptide synthetase polypeptide comprising a first peptide synthetase domain of a lipopeptide synthetase polypeptide, a second peptide synthetase domain of a lipopeptide synthetase polypeptide, wherein the second peptide synthetase domain is linked to a thioesterase domain of a peptide synthetase polypeptide.

The present invention also provides nucleic acids encoding the engineered polypeptides, host cells (e.g., bacterial cells, plant cells), and host organisms (e.g., plants) in which the engineered lipopeptide synthetase polypeptides are expressed. The present invention also provides methods for producing the engineered polypeptides.

The invention also provides novel lipopeptides, e.g., engineered lipopeptides produced by the engineered lipopeptide synthetases described herein. In certain embodiments, an engineered lipopeptide comprises one or more amino acid insertions, deletions, or substitutions relative to a naturally occurring lipopeptide (i.e., the novel lipopeptide is an analog of the naturally occurring lipopeptide). In certain embodiments, an engineered lipopeptide comprises one less amino acid than a corresponding naturally occurring lipopeptide. In certain embodiments, an engineered lipopeptide comprises a substitution of an amino acid relative to a naturally occurring form of the lipopeptide. In some embodiments, an engineered lipopeptide is a di-peptide linked to a fatty acid. In some embodiments, an engineered lipopeptide comprises a deletion of an N-terminal amino acid that is acylated in a naturally occurring form of the lipopeptide, and the N-terminal residue in the engineered lipopeptide is acylated. In some embodiments, an engineered lipopeptide comprises a fatty acid moiety that is not found on a naturally occurring lipopeptide.

The invention also provides methods of using engineered lipopeptide synthetase polypeptides, and lipopeptides produced by the synthetase polypeptides. In certain embodiments, lipopeptides are used as insecticidal agents. In certain embodiments, lipopeptides are used as antimicrobial (e.g., antifungal, antibacterial, antiviral, or antiprotazoal) agents. In certain embodiments, lipopeptides are used as surfactants. In certain embodiments, lipopeptides are used as food or feed additives (e.g., as a nutritional supplement). In certain embodiments, lipopeptides are incorporated into cosmetic compositions (e.g., for application to skin, hair, or nails). In certain embodiments, lipo-dipeptides (e.g., lipo-dipeptides that include a methionine residue) are used as food or feed additives or in cosmetic compositions.

In certain embodiments, an engineered polypeptide of the present invention produces a lipopeptide of interest. For example, an engineered polypeptide of the present invention may produce a surfactin analog having six amino acids. The surfactin analog may include an acyl moiety on an amino acid that is not acylated in native surfactin. Those of ordinary skill in the art will be able to use the teachings of the present invention to construct engineered polypeptides useful in the generation of any of a variety lipopeptides of interest. In certain embodiments, a lipopeptide of interest is produced in a commercially useful quantity.

In certain embodiments, an engineered lipopeptide synthetase polypeptide of the present invention is introduced into a host cell. Useful host cells encompassed by the present invention include, without limitation, bacterial hosts such as Bacillus subtilis. In certain embodiments, an engineered polypeptide of the present invention is introduced into a plant cell. Transgenic plants may be produced that comprise engineered polypeptides of the present invention, which transgenic plants exhibit one or more advantageous characteristics such as, without limitation, resistance to any of a variety of insect pests or microbial pathogens.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All cited patents, patent applications, and references (including references to public sequence database entries) are incorporated by reference in their entireties for all purposes. U.S. Provisional App. No. 60/923,679, filed Apr. 16, 2007, U.S. Provisional App. No. 60/945,527, filed Jun. 21, 2007, and U.S. Provisional App. No. 61/026,610, filed Feb. 6, 2008, are incorporated by reference in their entireties for all purposes.

The details of one or more embodiments of the invention are set forth in the description below. Other features, objects, and advantages of the invention will be apparent from the description and from the claims.

### BRIEF DESCRIPTION OF THE DRAWINGS

FIG. 1 is a schematic diagram of the surfactin synthetases, SrfA-A, SrfA-B, SrfA-C, and SrfA-TE. The amino acid encoded by the peptide synthetase domains of SrfA-A, SrfA-B, and SrfA-C are listed on each domain.

FIG. 2 shows MALDI spectra analysis of a sample from strain 14311_F6.

FIG. 3 shows MALDI spectra analysis of a sample from strain 14311_D3.

FIG. 4 shows MALDI spectra analysis of a sample from strain 15399-A1.

FIG. 5 shows MALDI spectra analysis of a sample from strain 15399-A1 or 1655-A1.

FIG. 6 is a schematic diagram of the structure of a surfactin analog with 7 amino acids and an analog produced by an engineered synthetase in which module 2 has been deleted.

FIG. 7 is a schematic diagram of the structure of a surfactin analog produced by an engineered synthetase in which module 1 has been deleted.

FIG. 8 shows MALDI spectra analysis of a sample from strain 15399-B6.

FIG. 9 shows MALDI spectra analysis of a sample from strain 15399-E5.

FIG. 10 shows MALDI spectra analysis of a sample from strain 15399-C6.

FIG. 11 shows a comparison of MALDI spectra analysis of samples from a strain that produces wild type surfactin (top) and strain 16923_G4 (bottom).

FIG. 12 shows a comparison of MALDI spectra analysis of samples from a strain that produces wild type surfactin (top) and strain 18499-B7 (bottom).

FIG. 13 shows MALDI spectra analysis of a sample from strain 16612_H2.

FIG. 14 shows MALDI spectra analysis of a sample from strain 16612_H2.

FIG. 15 shows MALDI spectra analysis of samples from strains expressing fatty acid (FA)-Glu-Leu.

FIG. 16 shows MALDI spectra analysis of samples from strains expressing FA-Glu-Leu.

FIG. 17 is a schematic diagram of an embodied strategy for engineering a FA-GLU-ASP construct.

FIG. 18 shows MALDI spectra analysis of a sample from a strain expressing FA-GLU-ASP-TE.

FIG. 19 shows MALDI spectra analysis of samples from strains expressing FA-GLU-ASP-TE-MG.

FIG. 20 shows MALDI spectra indicating the presence of surfactin (m/z=1074.7 (potassium adduct)) from the anaerobically grown Media E culture of Bacillus subtilis (strain OKB105 Δ(upp)Spect^{R}) (A), the low aeration M9YE culture of Bacillus subtilis (strain OKB105 Δ(upp)Spect^{R}) (B), the background spectra due to the MALDI matrix did not show a product at m/z=1074.7 (C).

FIG. 21 shows MALDI spectra indicating the presence of surfactin (m/z=1074.7 (potassium adduct)) from the anaerobically grown Media E culture of Bacillus subtilis (strain OKB105 Δ(upp)Spect^{R}) (A), the low aeration M9YE culture of Bacillus subtilis (strain OKB105 Δ(upp)Spect^{R}) (B), the background spectra due to the MALDI matrix did not show a product at m/z=1074.7 (C).

FIG. 22 shows MALDI spectra indicating the presence of FA-Glu-Leu (m/z=523.3 (sodium adduct) and 539.3 (potassium adduct) from the anaerobically grown Media E culture of Bacillus subtilis (27124-C1, strain OKB105 Δ(upp)Spect^{R} lacking modules 3-7 of wild-type surfactin synthetase) containing Media E with 80 g/L glucose (A), the anaerobically grown Media E culture of Bacillus subtilis (27124-C1, strain OKB105 Δ(upp)Spect^{R} lacking modules 3-7 of wild-type surfactin synthetase) containing Media E with 8 g/L ammonium nitrate (A) (B), and the background spectra due to the MALDI matrix did not show a product at m/z=539.3 (C).

### DESCRIPTION OF CERTAIN EMBODIMENTS

### Definitions

Beta-hydroxy fatty acid: The term “beta-hydroxy fatty acid” as used herein refers to a fatty acid chain comprising a hydroxy group at the beta position of the fatty acid chain. As is understood by those skilled in the art, the beta position corresponds to the third carbon of the fatty acid chain, the first carbon being the carbon of the carboxylate group. Thus, when used in reference to an acyl amino acid, where the carboxylate moiety of the fatty acid has been covalently attached to the nitrogen of the amino acid, the beta position corresponds to the carbon two carbons removed from the carbon having the ester group. A beta-hydroxy fatty acid to be used in accordance with the present invention may contain any number of carbon atoms in the fatty acid chain. As non-limiting examples, a beta-hydroxy fatty acid may contain 3, 4, 5, 6, 7, 8, 9, 10, 11, 12, 3, 14, 15, 15, 16, 17, 18, 19, 20 or more carbon atoms. Beta-hydroxy fatty acids to be used in accordance with the present invention may contain linear carbon chains, in which each carbon of the chain, with the exception of the terminal carbon atom and the carbon attached to the nitrogen of the amino acid, is directly covalently linked to two other carbon atoms. Additionally or alternatively, beta-hydroxy fatty acids to be used in accordance with the present invention may contain branched carbon chains, in which at least one carbon of the chain is directly covalently linked to three or more other carbon atoms. Beta-hydroxy fatty acids to be used in accordance with the present invention may contain one or more double bonds between adjacent carbon atoms. Alternatively, beta-hydroxy fatty acids to be used in accordance with the present invention may contain only single-bonds between adjacent carbon atoms. A non-limiting exemplary beta-hydroxy fatty acid that may be used in accordance with the present invention is beta-hydroxy myristic acid, which contains 13 to 15 carbons in the fatty acid chain. Those of ordinary skill in the art will be aware of various beta-hydroxy fatty acids that can be used in accordance with the present invention. Different beta-hydroxy fatty acid linkage domains that exhibit specificity for other beta-hydroxy fatty acids (e.g., naturally or non-naturally occurring beta-hydroxy fatty acids) may be used in accordance with the present invention to generate a lipopeptide of the practitioner's choosing.

Beta-hydroxy fatty acid linkage domain: The term “beta-hydroxy fatty acid linkage domain” as used herein refers to a polypeptide domain that covalently links a beta-hydroxy fatty acid to an amino acid to form an acyl amino acid. A variety of beta-hydroxy fatty acid linkage domains are known to those skilled in the art. However, different beta-hydroxy fatty acid linkage domains often exhibit specificity for one or more beta-hydroxy fatty acids. As one non-limiting example, the beta-hydroxy fatty acid linkage domain from surfactin synthetase is specific for the beta-hydroxy myristic acid, which contains 13 to 15 carbons in the fatty acid chain. Thus, the beta-hydroxy fatty acid linkage domain from surfactin synthetase can be used in accordance with the present invention to construct an engineered polypeptide useful in the generation of a lipopeptide that comprises the fatty acid beta-hydroxy myristic acid.

Characertistic sequence element: A “characteristic sequence element” refers to a a stretch of at least 4-500, 4-250, 4-100, 4-75, 4-50, 4-25, 4-15, or 4-10 amino acids that shows at least about 80%, 85%, 90%, 95%, 96%, 97%, 98%, 99%, or 100% identity with another polypeptide. In some embodiments, a characteristic sequence element participates in or confers function on a polypeptide.

Domain, Polypeptide domain: The terms “domain” and “polypeptide domain” as used herein generally refer to polypeptide moieties that naturally occur in longer polypeptides, or to engineered polypeptide moieties that are homologous to such naturally occurring polypeptide moieties, which polypeptide moieties have a characteristic structure (e.g., primary structure such as the amino acid sequence of the domain, although characteristic structure of a given domain also encompasses secondary, tertiary, quaternary, etc. structures) and/or exhibit one or more distinct functions. As will be understood by those skilled in the art, in many cases polypeptides are modular and are comprised of one or more polypeptide domains, each domain exhibiting one or more distinct functions that contribute to the overall function of the polypeptide. The structure and function of many such domains are known to those skilled in the art. For example, Fields and Song (Nature, 340(6230): 245-6, 1989) showed that transcription factors are comprised of at least two polypeptide domains: a DNA binding domain and a transcriptional activation domain, each of which contributes to the overall function of the transcription factor to initiate or enhance transcription of a particular gene that is under control of a particular promoter sequence. A polypeptide domain, as the term is used herein, also refers an engineered polypeptide that is homologous to a naturally occurring polypeptide domain.

Engineered: The term “engineered” as used herein refers to an entity that has been created or manipulated by the hand of man, and typically does not occur in nature. For example, in reference to a polypeptide, the term “engineered polypeptide” refers to a polypeptide that has been designed, produced, and/or manipulated by the hand of man; in most embodiments, the engineered polypeptide does not exist in nature. In various embodiments, an engineered polypeptide comprises two or more covalently linked polypeptide domains. Typically such domains will be linked via peptide bonds, although the present invention is not limited to engineered polypeptides comprising polypeptide domains linked via peptide bonds, and encompasses other covalent linkages known to those skilled in the art. One or more polypeptide domains of engineered polypeptides may be naturally occurring. In certain embodiments, engineered polypeptides of the present invention comprise two or more covalently linked domains, at least one of which is naturally occurring. In certain embodiments, two or more naturally occurring polypeptide domains are covalently linked to generate an engineered polypeptide. For example, naturally occurring polypeptide domains from two or more different polypeptides may be covalently linked to generate an engineered polypeptide. In certain embodiments, naturally occurring polypeptide domains of an engineered polypeptide are covalently linked in nature, but are covalently linked in the engineered polypeptide in a way that is different from the way the domains are linked nature. For example, two polypeptide domains that naturally occur in the same polypeptide but which are separated by one or more intervening amino acid residues may be directly covalently linked (e.g., by removing the intervening amino acid residues) to generate an engineered polypeptide of the present invention. Alternatively, or additionally, two polypeptide domains that naturally occur in the same polypeptide which are directly covalently linked together (e.g., not separated by one or more intervening amino acid residues) may be indirectly covalently linked (e.g., by inserting one or more intervening amino acid residues) to generate an engineered polypeptide of the present invention. In certain embodiments, one or more covalently linked polypeptide domains of an engineered polypeptide may not exist naturally. For example, such polypeptide domains may be engineered themselves. In some embodiments, a polypeptide domain includes a first portion derived from a first naturally occurring polypeptide, and a second portion derived from a second naturally occurring polypeptide (i.e., the polypeptide domain is a hybrid domain).

Fatty acid linkage domain: The term “fatty acid linkage domain” as used herein refers to a polypeptide domain that covalently links a fatty acid to an amino acid to form an acyl amino acid. A variety of fatty acids are known to those of ordinary skill in the art, as are a variety of fatty acid linkage domains, such as for example, fatty acid linkage domains present in various peptide synthetase complexes that produce lipopeptides. In certain embodiments, a fatty acid linkage domain of the present invention comprises a beta-hydroxy fatty acid linkage domain. In some embodiments, an engineered beta-hydroxy fatty acid linkage domain as described herein is homologous to a naturally occuring beta-hyroxy fatty acid linkage domain.

Homologous: “Homologous”, as the term is used herein, refers to the characteristic of being similar at the nucleotide or amino acid level to a reference nucleotide or polypeptide. For example, a polypeptide domain that has been altered at one or more positions such that the amino acids of the reference polypeptide have been substituted with amino acids exhibiting similar biochemical characteristics (e.g., hydrophobicity, charge, bulkiness) will generally be homologous to the reference polypeptide. Percent identity and similarity at the nucleotide or amino acid level are often useful measures of whether a given nucleotide or polypeptide is homologous to a reference nucleotide or amino acid. Those skilled in the art will understand the concept of homology and will be able to determine whether a given nucleotide or amino acid sequence is homologous to a reference nucleotide or amino acid sequence. In certain embodiments, a polypeptide (including a polypeptide domain or moiety) is considered to be homologous to a corresponding other polypeptide (e.g., a corresponding naturally occurring polypeptide) if it shows at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more overall sequence identity and/or shares at least one characteristic sequence element. In some embodiments, a polypeptide is homologous to another polypeptide if it shows both a specified degree of overall sequence identity and a characteristic sequence element.

Lipopeptide: The term “lipopeptide” as used herein refers to a peptide of two or more amino acids that is covalently linked to a fatty acid. In certain embodiments, lipopeptides according to the present invention comprise a beta-hydroxy fatty acid. In certain embodiments, lipopeptides comprise a beta-amino fatty acid. In certain embodiments, lipopeptides are produced according to the present invention by an engineered lipopeptide synthetase. For example, in some embodiments, lipopeptides are produced by engineered lipopeptide synthetase polypeptides comprising a deletion, relative to a corresponding naturally occurring lipopeptide synthetase, such that the engineered synthetase polypeptide produces a lipopeptide having one less amino acid than the corresponding naturally occurring lipopeptide synthetase. In some embodiments, the deletion is, for example, a deletion of at least a portion of a condensation (C) domain, adenylation (A) domain, or thioloation (T) domain. In certain embodiments, the present invention provides compositions and methods for producing lipopeptides by employing engineered polypeptides comprising a first peptide synthetase domain of a first lipopeptide synthetase polypeptide, and a second peptide synthetase domain of a second peptide synthetase polypeptide (e.g., a second nonribosomal peptide synthetase, e.g., a lipopeptide synthetase). The first and second peptide synthetase domains are covalently linked such that the engineered polypeptide produces a lipopeptide comprising an amino acid encoded by the first peptide synthetase domain linked to an amino acid encoded by the second peptide synthetase domain. In certain embodiments, the present invention provides compositions and methods for producing lipopeptides by employing engineered polypeptides comprising a first peptide synthetase domain of a naturally occurring lipopeptide synthetase polypeptide and a second peptide synthetase domain of a naturally occurring peptide synthetase polypeptide (e.g., a lipopeptide synthetase polypeptide), covalently linked to a thioesterase domain of a peptide synthetase polypeptide.

Engineered lipopeptide synthetase polypeptides described herein often include a peptide synthetase domain comprising a fatty acid linkage domain. Typically, the identity of the amino acid moiety of the fatty acid linked amino acid (also referred to herein as an acyl amino acid) is determined by the amino acid specificity of the peptide synthetase domain. For example, the peptide synthetase domain may specify any one of the naturally occurring amino acids known by those skilled the art to be used in ribosome-mediated polypeptide synthesis. Alternatively, a peptide synthetase domain may specify a non-naturally occurring amino acid, e.g., a modified amino acid or amino acid analog. Similarly, the identity of the fatty acid moiety of the acyl amino acid is determined by the fatty acid specificity of the fatty acid linkage domain, such as for example a fatty acid linkage domain that is specific for a beta-hydroxy fatty acid. For example, the beta-hydroxy fatty acid may be any of a variety of naturally occurring or non-naturally occurring beta-hydroxy fatty acids. In some embodiments, an engineered beta-hydroxy fatty acid linkage domain as described herein is homologous to a naturally occuring beta-hyroxy fatty acid linkage domain.

Naturally occurring: The term “naturally occurring”, as used herein when referring to an amino acid, refers to one of the standard group of twenty amino acids that are the building blocks of polypeptides of most organisms, including alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, the term “naturally occurring” also refers to amino acids that are used less frequently and are typically not included in this standard group of twenty but are nevertheless still used by one or more organisms and incorporated into certain polypeptides. For example, the codons UAG and UGA normally encode stop codons in most organisms. However, in some organisms the codons UAG and UGA encode the amino acids selenocysteine and pyrrolysine. Thus, in certain embodiments, selenocysteine and pyrrolysine are naturally occurring amino acids. The term “naturally occurring”, as used herein when referring to a polypeptide or polypeptide domain, refers to a polypeptide or polypeptide domain that occurs in one or more organisms. Certain naturally-occurring polypeptides are exemplified herein. Others can be found in public databases such as the GenBank® and EMBL databases. In certain embodiments, engineered polypeptides of the present invention comprise one or more naturally occurring polypeptide domains that naturally exist in different polypeptides. In certain embodiments, engineered polypeptides of the present invention comprise two or more naturally occurring polypeptide domains that are covalently linked (directly or indirectly) in the polypeptide in which they occur, but are linked in the engineered polypeptide in a non-natural manner. As a non-limiting example, two naturally occurring polypeptide domains that are directly covalently linked may be separated in the engineered polypeptide by one or more intervening amino acid residues. Additionally or alternatively, two naturally occurring polypeptide domains that are indirectly covalently linked may be directly covalently linked in the engineered polypeptide, e.g. by removing one or more intervening amino acid residues. Such engineered polypeptides are not naturally occurring, as the term is used herein.

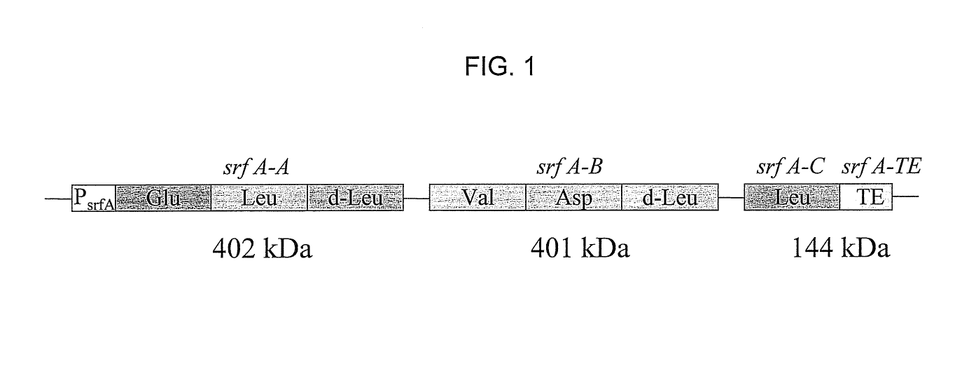

Peptide synthetase complex: The term “peptide synthetase complex” as used herein refers to an enzyme that catalyzes the non-ribosomal production of peptides or set of peptides. A peptide synthetase complex may comprise a single enzymatic subunit (e.g., a single polypeptide), or may comprise two or more enzymatic subunits (e.g., two or more polypeptides). A peptide synthetase complex typically comprises at least one peptide synthetase domain, and may further comprise one or more additional domains such as for example, a fatty acid linkage domain, a thioesterase domain, a reductase domain, etc. Peptide synthetase domains of a peptide synthetase complex may comprise two or more enzymatic subunits, with two or more peptide synthetase domains present in a given enzymatic subunit. For example the surfactin peptide synthetase complex (also referred to herein simply as “surfactin synthetase complex”) comprises three distinct polypeptide enzymatic subunits: the first two subunits comprise three peptide synthetase domains, while the third subunit comprises a single peptide synthetase domain. FIG. 1 is a schematic diagram of the surfactin synthetases, SrfA-A, SrfA-B, SrfA-C, and SrfA-TE (which contains a thioesterase domain). The amino acid encoded by the peptide synthetase domains of SrfA-A, SrfA-B, and SrfA-C are listed on each domain.

Peptide synthetase domain: The term “peptide synthetase domain” as used herein refers to a polypeptide domain that minimally comprises two subdomains: an adenylation (A) domain, responsible for selectively recognizing and activating a specific amino acid, and a thiolation (T) domain, which tethers the activated amino acid to a cofactor via thioester linkage. Peptide synthetase domains can also include a condensation (C) domain, which links amino acids joined to successive units of the peptide synthetase by the formation of amide bonds. Peptide synthetase domains can also include a fatty acid linkage domain.

An A domain has approximately 550 amino acid residues. A domains of nonribosomal polypeptide synthetases typically share 30%-60% overall identity with each other and take on a characteristic three dimensional fold that is similar to that of firefly luciferase (Weber and Marahiel, Struct. 9:R3-R9, 2001). A domains include highly conserved core motifs which are described in Stachelhaus et al., Chem. Biol. 6(8):493-505. In some embodiments, such T domains (also known as peptidyl carrier domains, or PCP domains) have approximately 100 amino acids and are located C-terminal to A domains. T domains have a conserved sequence motif: (L/I)GG(D/H)S(L/I)(SEQ ID NO:______) and have a similar three dimensional structure to that of acyl carrier proteins of fatty acid and polyketide synthetases (Weber and Marahiel, Struct. 9:R3-R9, 2001). C domains have approximately 450 amino acid residues including a conserved HHXXXDG (SEQ ID NO:______) motif, and are located at the N-terminus of peptide synthetase domains. A table listing conserved motifs in A, T, and C domains of lipopeptide synthetases is found in Lin et al., J. Bacter. 181(16):5060-5067, 1999. Conserved motifs are also disclosed in Marahiel et al., Chem. Rev. 97:2651-2673, 1997.

A peptide synthetase domain typically recognizes and activates a single amino acid, and in the situation where the peptide synthetase domain is not the first domain in the pathway, links the specific amino acid to the growing peptide chain. Some peptide synthetase domains are specific for a single amino acid (e.g., a domain incorporates only Glu residues). Some peptide synthetase domains show relaxed specificity and will incorporate more than one type of amino acid (e.g., a domain incorporates a Glu residue, or another amino acid). A variety of peptide synthetase domains are known to those skilled in the art, e.g. such as those present in a variety of nonribosomal peptide synthetase complexes. Those skilled in the art will be aware of methods to determine whether a give polypeptide domain is a peptide synthetase domain. Different peptide synthetase domains often exhibit specificity for one or more amino acids. As one non-limiting example, the first peptide synthetase domain from the surfactin synthetase Srf-A subunit is specific for glutamate. Thus, the peptide synthetase domain from surfactin synthetase can be used in accordance with the present invention to construct an engineered polypeptide useful in the generation of a lipopeptide that comprises the amino acid glutamate. Different peptide synthetase domains that exhibit specificity for other amino acids (e.g., naturally or non-naturally occurring amino acids) may be used in accordance with the present invention to generate a lipopeptide of the practitioner's choosing. Herein, the term “peptide synthetase domain” is used interchangeably with the “module” or “peptide synthetase module”.

Polypeptide: The term “polypeptide” as used herein refers to a series of amino acids joined together in peptide linkages, such as polypeptides synthesized by ribosomal machinery in naturally occurring organisms. The term “polypeptide” also refers to a series of amino acids joined together by non-ribosomal machinery, such as by way of non-limiting example, polypeptides synthesized by various peptide synthetases. Such non-ribosomally produced polypeptides exhibit a greater diversity in covalent linkages than polypeptides synthesized by ribosomes (although those skilled in the art will understand that the amino acids of ribosomally-produced polypeptides may also be linked by covalent bonds that are not peptide bonds, such as the linkage of cystines via di-sulfide bonds). For example, surfactin is a lipopeptide synthesized by the surfactin synthetase complex. Surfactin comprises seven amino acids, which are initially joined by peptide bonds, as well as a beta-hydroxy fatty acid covalently linked to the first amino acid, glutamate. However, upon addition the final amino acid (leucine), the polypeptide is released and the thioesterase domain of the SRFC protein catalyzes the release of the product via a nucleophilic attack of the beta-hydroxy of the fatty acid on the carbonyl of the C-terminal Leu of the peptide, cyclizing the molecule via formation of an ester, resulting in the C-terminus carboxyl group of leucine attached via a lactone bond to the beta-hydroxyl group of the fatty acid. Polypeptides can be two or more amino acids in length, although most polypeptides produced by ribosomes and peptide synthetases are longer than two amino acids. For example, polypeptides may be 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500 or more amino acids in length.

Reductase Domain: The term “reductase domain” as used herein refers to a polypeptide domain that catalyzes release of a lipopeptide produced by a peptide synthetase complex from the peptide synthetase complex. In certain embodiments, a reductase domain is covalently linked to peptide synthetase domains and a fatty acid linkage domain such as a beta-hydroxy fatty acid linkage domain to generate an engineered polypeptide useful in the synthesis of a lipopeptide. A variety of reductase domains are found in nonribosomal peptide synthetase complexes from a variety of species. A non-limiting example of a reductase domain that may be used in accordance with the present invention includes the reductase domain from linear gramicidin (ATCC8185). However, any reductase domain that releases a lipopeptide produced by a peptide synthetase complex from the peptide synthetase complex may be used in accordance with the present invention. Reductase domains are typically characterized by the presence of the consensus sequence: [LIVSPADNK]-x(9)-{P}-x(2)-Y-[PSTAGNCV]-[STAGNQCIVM]-[STAGC]-K- {PC}-[SAGFYR]-[LIVMSTAGD]-x-{K}-[LIVMFYW]-{D}-x-{YR}-[LIVMFYWGAPTHQ]-[GSACQRHM] (SEQ ID NO:______), where square brackets (“[ ]”) indicate amino acids that are typically present at that position, squiggly brackets (“{ }”) indicate amino acids that amino acids that are typically not present at that position, and “x” denotes any amino acid or a gap. X(9) for example denotes any amino acids or gaps for nine consecutive positions. Those skilled in the art will be aware of methods to determine whether a given polypeptide domain is a reductase domain.

Thioesterase domain: The term “thioesterase domain” as used herein refers to a polypeptide domain that catalyzes release of an acyl amino acid produced by a peptide synthetase complex from the peptide synthetase complex. In certain embodiments, a thioesterase domain is covalently linked to peptide synthetase domains and a fatty acid linkage domain such as a beta-hydroxy fatty acid linkage domain to generate an engineered polypeptide useful in the synthesis of a lipopeptide. A variety of thioesterase domains are found in nonribosomal peptide synthetase complexes from a variety of species. A non-limiting example of a thioesterase domain that may be used in accordance with the present invention includes the thioesterase domain from the Bacillus subtilis surfactin synthetase complex, present in Srf-C subunit. However, any thioesterase domain that releases a lipopeptide produced by a peptide synthetase complex from the peptide synthetase complex may be used in accordance with the present invention. Thioesterase domains are typically characterized by the presence of the consensus sequence: [LIV]-{KG}-[LIVFY]-[LIVMST]-G-[HYWV]-S-{YAG}-G-[GSTAC] (SEQ ID NO:______), where square brackets (“[ ]”) indicate amino acids that are typically present at that position, and squiggly brackets (“{ }”) indicate amino acids that amino acids that are typically not present at that position. Those skilled in the art will be aware of methods to determine whether a given polypeptide domain is a thioesterase domain.

### Lipopeptide Synthetase Complexes

Peptide synthetase complexes are multienzymatic complexes found in both prokaryotes and eukaryotes comprising one or more enzymatic subunits that catalyze the non-ribosomal production of a variety of peptides (see, for example, Kleinkauf et al., Annu Rev. Microbiol. 41:259-289, 1987; see also U.S. Pat. No. 5,652,116 and U.S. Pat. No. 5,795,738). Non-ribosomal synthesis is also known as thiotemplate synthesis (see e.g., Kleinkauf et al.). Peptide synthetase complexes typically include one or more peptide synthetase domains that recognize specific amino acids and are responsible for catalyzing addition of the amino acid to the polypeptide chain. Lipopeptide synthetase complexes are peptide synthetase complexes that produce a peptide that includes an acyl amino acid as part of the peptide chain.

The catalytic steps in the addition of amino acids include: recognition of an amino acid by the peptide synthetase domain, activation of the amino acid (formation of an amino-acyladenylate), binding of the activated amino acid to the enzyme via a thioester bond between the carboxylic group of the amino acid and an SH group of an enzymatic co-factor, which cofactor is itself bound to the enzyme inside each peptide synthetase domain, and formation of the peptide bonds among the amino acids. A peptide synthetase domain comprises subdomains that carry out specific roles in these steps to form the peptide product. One subdomain, the adenylation (A) domain, is responsible for selectively recognizing and activating the amino acid that is to be incorporated by a particular unit of the peptide synthetase. The activated amino acid is joined to the peptide synthetase through the enzymatic action of another subdomain, the thiolation (T) domain, that is generally located adjacent to the A domain. Amino acids joined to successive units of the peptide synthetase are subsequently linked together by the formation of amide bonds catalyzed by another subdomain, the condensation (C) domain.

Peptide synthetase domains that catalyze the addition of D-amino acids also have the ability to catalyze the racemization of L-amino acids to D-amino acids. Peptide synthetase complexes also typically include a conserved thioesterase domain that terminates the growing amino acid chain and releases the product.

Genes that encode peptide synthetase complexes typically have a modular structure that parallels the functional domain structure of the complexes (see, for example, Cosmina et al., Mol. Microbiol. 8:821, 1993; Kratzxchmar et al., J. Bacteriol. 171:5422, 1989; Weckermann et al., Nuc. Acids res. 16:11841, 1988; Smith et al., EMBO J. 9:741, 1990; Smith et al., EMBO J. 9:2743, 1990; MacCabe et al., J. Biol. Chem. 266:12646, 1991; Coque et al., Mol. Microbiol. 5:1125, 1991; Diez et al., J. Biol. Chem. 265:16358, 1990).

Hundreds of peptides are known to be produced by peptide synthetase complexes. Such nonribosomally-produced peptides often have non-linear structures, including cyclic structures exemplified by the peptides surfactin, cyclosporin, tyrocidin, and mycobacillin, or branched cyclic structures exemplified by the peptides polymyxin and bacitracin. Moreover, such nonribosomally-produced peptides may contain amino acids not usually present in ribosomally-produced polypeptides such as for example norleucine, beta-alanine and/or ornithine, as well as D-amino acids. Additionally or alternatively, such nonribosomally-produced peptides may comprise one or more non-peptide moieties that are covalently linked to the peptide. Nonribosomal lipopeptide synthetases described herein produce peptides that include a fatty acid. As one non-limiting example, surfactin is a cyclic lipopeptide that comprises a beta-hydroxy fatty acid covalently linked to the first glutamate of the lipopeptide. Other non-peptide moieties that are covalently linked to peptides produced by peptide synthetase complexes are known to those skilled in the art, including for example sugars, chlorine or other halogen groups, N-methyl and N-formyl groups, glycosyl groups, acetyl groups, etc.

Typically, each amino acid of a non ribosomally-produced peptide is specified by a distinct peptide synthetase domain. For example, the surfactin synthetase complex which catalyzes the polymerization of the lipopeptide surfactin consists of three enzymatic subunits (FIG. 1). The first two subunits each comprise three peptide synthetase domains, whereas the third has only one. These seven peptide synthetase domains are responsible for the recognition, activation, binding and polymerization of L-Glu, L-Leu, D-Leu, L-Val, L-Asp, D-Leu and L-Leu, the amino acids present in surfactin.

A similar organization in discrete, repeated peptide synthetase domains occurs in various peptide synthetase genes in a variety of species, including bacteria and fungi, for example srfA (Cosmina et al., Mol. Microbiol. 8, 821-831, 1993), grsA and grsB (Kratzxchmar et al., J. Bacterial. 171, 5422-5429, 1989) tycA and tycB (Weckermann et al., Nucl. Acid. Res. 16, 11841-11843, 1988) and ACV from various fungal species (Smith et al., EMBO J. 9, 741-747, 1990; Smith et al., EMBO J. 9, 2743-2750, 1990; MacCabe et al., J. Biol. Chem. 266, 12646-12654, 1991; Coque et al., Mol. Microbiol. 5, 1125-1133, 1991; Diez et al., J. Biol. Chem. 265, 16358-16365, 1990). The peptide synthetase domains of even distant species contain sequence regions with high homology, some of which are conserved and specific for all the peptide synthetases. Additionally, certain sequence regions within peptide synthetase domains are even more highly conserved among peptide synthetase domains which recognize the same amino acid (Cosmina et al., Mol. Microbiol. 8, 821-831, 1992). Specific lipopeptides and lipopeptide synthetases are described below. Additional lipopeptides and corresponding synthetases are known in the art.

Surfactin and Surfactin Synthetase

Surfactin is cyclic lipopeptide that is naturally produced by certain bacteria, including the Gram-positive endospore-forming bacteria Bacillus subtilis. Surfactin is an amphiphilic molecule (having both hydrophobic and hydrophilic properties) and is thus soluble in both organic solvents and water. Surfactin exhibits exceptional surfactant properties, making it a commercially valuable molecule.

Due to its surfactant properties, surfactin also functions as an antibiotic. For example, surfactin is known to be effective as an anti-bacterial, anti-viral, anti-fungal, anti-mycoplasma and hemolytic compound.

An anti-bacterial compound, surfactin it is capable of penetrating the cell membranes of all types of bacteria, including both Gram-negative and Gram-positive bacteria, which differ in the composition of their membrane. Gram-positive bacteria have a thick peptidoglycan layer on the outside of their phospholipid bilayer. In contrast, Gram-negative bacteria have a thinner peptidoglycan layer on the outside of their phospholipid bilayer, and further contain an additional outer lipopolysaccharide membrane. Surfactin's surfactant activity permits it to create a permeable environment for the lipid bilayer and causes disruption that solubilizes the membrane of both types of bacteria. In order for surfactin to carry out minimal antibacterial effects, the minimum inhibitory concentration (MIC) is in the range of 12-50 μg/ml.

In addition to its antibacterial properties, surfactin also exhibits antiviral properties, and its known to disrupt enveloped viruses such as HIV and HSV. Surfactin not only disrupts the lipid envelope of viruses, but also their capsids through ion channel formations. Surfactin isoforms containing fatty acid chains with 14 or 15 carbon atoms exhibited improved viral inactivation, thought to be due to improved disruption of the viral envelope.

Surfactin consists of a seven amino acid peptide loop, and a hydrophobic fatty acid chain (beta-hydroxy myristic acid) that is thirteen to fifteen carbons long. The fatty acid chain allows permits surfactin to penetrate cellular membranes. Surfactin is synthesized by the surfactin synthetase complex, which comprises the three surfactin synthetase polypeptide subunits SrfA-A, SrfA-B, and SrfA-C. The surfactin synthetase polypeptide subunits SrfA-A and SrfA-B each comprise three peptide synthetase domains, each of which adds a single amino acid to the growing surfactin peptide, while the monomodular surfactin synthetase polypeptide subunit SrfA-C comprises a single peptide synthetase domain and adds the last amino acid residue to the heptapeptide. Additionally the SrfA-C subunit comprises a thioesterase domain, which catalyzes the release of the product via a nucleophilic attack of the beta-hydroxy of the fatty acid on the carbonyl of the C-terminal Leu of the peptide, cyclizing the molecule via formation of an ester. The spectrum of the beta-hydroxy fatty acids was elucidated as iso, anteiso C13, iso, normal C14 and iso, anteiso C15, and a recent study has indicated that surfactin retains an R configuration at C-beta (Nagai et al., Study on surfactin, a cyclic depsipeptide. 2. Synthesis of surfactin B2 produced by Bacillus natto KMD 2311. Chem Pharm Bull (Tokyo) 44: 5-10, 1996).

Fengycin and Fengycin Synthetases

Fengycin, naturally produced by Bacillus subtilis, is a cyclic lipopeptide which is active against phytopathogenic fungi and the larvae of the cabbage white butterfly (Pieris rapae crucivora)(Kim et al., J Appl Microbiol. 97(5):942-9, 2004). Fengycin has the following amino acids: L-Glu, D-Orn, L-Tyr, D-allo-Thr, L-Glu, D-Ala, L-Pro, L-Glu, D-Tyr, L-Ile, with a lactone bond connecting L-Tyr and L-Ile. The fengycin synthetase complex includes products of five synthetase genes, fenC, fedD, fedE, fedA, and fenB (Lin et al., J. Bacteriol. 181(16):5060-5067, 1999).

Arthrofactin and Arthofactin Synthetases

Arthrofactin is a cyclic lipopeptide naturally produced by Pseudomonas sp. MIS38. Arthofactin has potent surfactant properties. The three arthofactin synthase genes, ArfA, ArfB, and ArfC, encode two, four, and five modules, respectively, each of which has a condensation, adenylation, and thiolation domain (Roongsawang, et al., Chem. Biol. 10:869-880, 2003). Arthofactin has eleven amino acids in the following sequence: D-Leu, D-Asp, D-Thr, D-Leu, D-Leu, D-Ser, L-Leu, D-Ser, L-Ile, L-Ile, L-Asp.

Lichenysins and Lichenysin Synthetases

Lichenysins are lipopeptides naturally produced by Bacillus licheniformis strains. Lichenysins have seven amino acids in the following sequence: L-Glx, L-Leu, D-Leu, L-Val, L-Asx, D-Leu, L-Ile/Leu/Val. The first amino acid is connected to a β-hydroxyl fatty acid, and the C-terminal amino acid forms a lactone ring to the β-OH of the lipophilic part of the molecule. Lichenysins are produced by a synthetase complex encoded by three genes, licA, licB, and licE (Konz et al., J. Bacteriol. 181(1):133-140, 1999).

Other examples of lipopeptides are known and include iturins, plipastatin, agrastatin, daptomycin, syringomycin, bacillomycins, esperin, mycosubtilin, bacillomycin F, and surfactant 86.

Table 1 provides a list of exemplary naturally occurring lipopeptide synthetase polypeptides, including GenBank® Accession numbers for the amino acid sequences of the polypeptides, domains (modules), and amino acids encoded by each domain of the polypeptides. Typically, the first module of the first synthetase polypeptide in a synthetase complex includes a fatty acid linkage domain (e.g., module 1 of SrfA-A, module 1 of FenC, module 1 of ArfA, module 1 of SyrE, and so forth).

TABLE 1
Exemplary naturally occurring lipopeptide synthetase polypeptides

| | | | Polypeptide | | |
|---|---|---|---|---|---|
| Row | Gene | Polypeptide Name | Accession No. | Synthetase | Amino |
| No. | Name | Species origin | GI No. | domain | Acid* |
| 1 | SrfAA | Surfactin synthetase | NP_388230 | 1 | L-Glu |
| | | Bacillus subtilis | GI: 16077417 | 2 | L-Leu |
| | | | | 3 | D-Leu |
| 2 | SrfAB | Surfactin synthetase | NP_388231 | 4 | L-Val |
| | | Bacillus subtilis | GI: 16077418 | 5 | L-Asp |
| | | | | 6 | D-Leu |
| 3 | SrfAC | Surfactin synthetase | NP_388233 | 1 | L-Leu |
| | | Bacillus subtilis | GI: 16077420 | | |
| 4 | FenC | Fengycin synthase | AAC36721 | 1 | L-Glu |
| | | Bacillus subtilis | GI: 3643187 | 2 | D-Orn |
| 5 | FenD | Fengycin synthase | EMBL Acc. No.: | 3 | L-Tyr |
| | | Bacillus subtilis | AJ011849 | 4 | D-allo-Thr |
| 6 | FedE | Fengycin synthase | AAB80956.1 | 5 | L-Glu |
| | | Bacillus subtilis | GI: 2522214 | 6 | D-Ala or |
| | | | | | D-val |
| 7 | FenA | Fengycin synthase | AAB80955.2 | 7 | L-Pro |
| | | Bacillus subtilis | GI: 37577047 | 8 | L-Glu |
| | | | | 9 | D-Tyr |
| 8 | FenB | Fengycin synthase | AAB00093.1 | 10 | L-Ile |
| | | Bacillus subtilis | GI: 840624 | | |
| 9 | ArfA | Arthrofactin synthetase A | BAC67534.2 | 1 | D-Leu |
| | | Pseudomonas sp. MIS38 | GI: 32968220 | 2 | D-Asp |
| 10 | ArfB | Arthrofactin synthetase B | BAC67535.1 | 3 | D-Thr |
| | | Pseudomonas sp. MIS38 | GI: 29501267 | 4 | D-Leu |
| | | | | 5 | D-Leu |
| | | | | 6 | D-Ser |
| 11 | ArfC | Arthrofactin synthetase C | BAC67536.1 | 7 | L-Leu |
| | | Pseudomonas sp. MIS38 | GI: 29501268 | 8 | D-Ser |
| | | | | 9 | L-Ile |
| | | | | 10 | L-Ile |
| | | | | 11 | L-Asp |
| 12 | LicA | lichenysin synthetase A | YP_090052.1 | 1 | L-Gln |
| | | Bacillus licheniformis | GI: 52784223 | 2 | L-Leu |
| | | | | 3 | D-Leu |
| 13 | LicB | lichenysin synthetase B | YP_090053.1 | 4 | L-Val |
| | | Bacillus licheniformis | GI: 52784224 | 5 | L-Asp |
| | | | | 6 | D-Leu |
| 14 | LicC | lichenysin synthetase C | YP_090054.1 | 7 | L-Ile |
| | | Bacillus licheniformis | GI: 52784225 | | |
| 15 | SyrE | syringomycin synthetase | AAC80285.1 | 1 | Ser |
| | | Pseudomonas syringae pv. syringae | GI: 3510629 | 2 | D-Ser |
| | | | | 3 | D-Dab |
| | | | | 4 | Dab |
| | | | | 5 | Arg |
| | | | | 6 | Phe |
| | | | | 7 | Dhb |
| | | | | 8 | Asp |
| 16 | SyrB1 | syringomycin biosynthesis enzyme 1 | AAA85160.2 | 9 | Thr |
| | | Pseudomonas syringae pv. syringa | GI: 5748807 | | |
| 17 | SypA | syringopeptin synthetase | AAF99707.2 | 1 | L-Dhb |
| | | Pseudomonas syringae pv. syringae | GI: 29165622 | 2 | D-Pro |
| | | | | 3 | D-Val |
| | | | | 4 | L-Val |
| | | | | 5 | D-Ala |
| 18 | SypB | syringopeptin synthetase B | AAO72424.1 | 6 | D-Ala |
| | | Pseudomonas syringae pv. syringae | GI: 29165623 | 7 | D-Val |
| | | | | 8 | D-Val |
| | | | | 9 | L-Dhb |
| | | | | 10 | D-Ala |
| 19 | SypC | syringopeptin synthetase C | AAO72425.1 | 11 | D-Val |
| | | Pseudomonas syringae pv. syringae | GI: 29165624 | 12 | L-Ala |
| | | | | 13 | D-Ala |
| | | | | 14 | D-Dhb |
| | | | | 15 | L-allo-Thr |
| | | | | 16 | L-Ser |
| | | | | 17 | L-Ala |
| | | | | 18 | D-Dhb |
| | | | | 19 | D-Ala |
| | | | | 20 | D-Dab |
| | | | | 21 | D-Dab |
| | | | | 22 | D-Tyr |
| 20 | ItuA | iturin A synthetase A | BAB69698.1 | 1 | Asn |
| | | Bacillus subtilis | GI: 16040970 | | |
| 21 | ItuB | iturin A synthetase B | BAB69699.1 | 2 | Tyr |
| | | Bacillus subtilis | GI: 16040971 | 3 | Asn |
| | | | | 4 | Gln |
| | | | | 5 | Pro |
| 22 | ItuC | iturin A synthetase C | BAB69700.1 | 6 | Asn |
| | | Bacillus subtilis | GI: 16040972 | 7 | Ser |

*Dab = 2,4,-diaminobutyric acid, Dhb = 2,3-dehydroaminobutyric acid

Engineered Polypeptides Useful in the Generation of Lipopeptides

The present invention provides compositions and methods for the generation of lipopeptides. In certain embodiments, compositions of the present invention comprise engineered polypeptides that are useful in the production of analogs of lipopeptides naturally produced by a cell (e.g., by a cell of a microorganism). The engineered polypeptides include deletion and module substitution mutants of naturally occurring lipopeptide synthetase polypeptides.

Engineered polypeptides Having Deletions

In certain embodiments, an engineered lipopeptide synthetase polypeptide is a deletion mutant of a naturally occurring lipopeptide synthetase polypeptide, wherein the corresponding naturally occurring polypeptide comprises a first and second peptide synthetase domain, and wherein one or both peptide synthetase domains comprises a condensation domain (C domain), and wherein both peptide synthetase domains include an adenylation domain (A domain), and a thiolation domain (T domain). In some embodiments, the engineered polypeptide includes a deletion of at least a portion of a C domain, a portion of an A domain, or a portion of a T domain, relative to the naturally occurring lipopeptide synthetase polypeptide. In some embodiments, the engineered lipopeptide polypeptide also includes a fatty acid linkage domain.

In certain embodiments, an engineered polypeptide produces a lipopeptide having a fewer (e.g., one less) amino acid than a lipopeptide produced by the naturally occurring polypeptide, when expressed under conditions in which the naturally occurring polypeptide produces the naturally occurring lipopeptide (e.g., when expressed in a cell with other members of the peptide synthetase complex from which the engineered polypeptide is derived). In certain embodiments, an engineered polypeptide is a surfactin synthetase A-A polypeptide (SrfA-A), and is expressed in a cell with other members of the surfactin synthetase complex (e.g., SrfA-B and SrfA-C), under conditions in which the synthetase complex produces a lipopeptide.

In certain embodiments, an engineered polypeptide comprises a deletion of at least a C domain and an A domain, relative to the naturally occurring form of the lipopeptide synthetase polypeptide. For example, an engineered polypeptide may comprise a deletion of a C domain and A domain of the second peptide synthetase domain. In certain embodiments, an engineered polypeptide comprises a C domain and A domain of the first peptide synthetase domain, fused to a T domain which is a hybrid T domain comprising a portion of the T domain from the first peptide synthetase domain (T1), and a portion of the T domain from the second peptide synthetase domain (T2). For example, the T domain is a hybrid T domain containing an N-terminal portion of T1 joined to a C-terminal portion of T2. In certain embodiments, portions of T1 and T2 are joined in a homologous region. An example of a homologous region in thiolation domains of SrfA is shown in Table 2 below. In some embodiments, the engineered polypeptide is produced by engineering a cell in which genomic DNA encoding homologous regions of T domains of the first and second modules have been joined by deletion of the intervening region.

In addition to engineered polypeptides as described above, we have discovered that analogs of natural lipopeptides can be produced by deleting the A and T domains of a first peptide synthetase domain (first module) of a lipopeptide synthetase, and joining a portion of the C domain of the first module to a portion of the C domain of the second module (i.e., to create a C domain which is a hybrid C domain containing residues from the first module and residues from the second module). In certain embodiments, the hybrid C domain contains an N-terminal portion of a first C domain (C1) joined to a a C-terminal portion of a second C domain (C2). In certain embodiments, the portions of C1 and C2 are joined in a highly variable region. In certain embodiments, the C domains are C domains of modules 1 and 2 of SrfA-A, and the C domains are joined in a region that is bounded by the fusion point upstream and downstream sequences shown in Tables 5 and 6 below.

When a lipopeptide synthetase polypeptide is engineered in this manner, it produces a lipopeptide in which a fatty acid is linked the first amino acid of the peptide, and wherein the first amino acid of the peptide is the amino acid specified by module 2. By way of example, this was performed with SrfA-A (see Example 3). A deletion mutant of SrfA-A was constructed in which a portion of the C domain of module 1 was joined to a portion of the C domain of module 2, and the intervening amino acids were absent. In this example, the engineered SrfA polypeptide produced a cyclic, six membered surfactin analog containing an N-terminal acylated leucine.

Accordingly, the present invention demonstrates that one may provide an engineered lipopeptide synthetase polypeptide that includes the N-terminal region of the first module that directs linkage of a fatty acid to an amino acid. The engineered polypeptide can link a fatty acid to an amino acid specified by the second module in the natural polypeptide. We discovered that the when the engineered lipopeptide is expressed in a cell that includes other members of the lipopeptide synthetase complex, the analog is produced, indicating that downstream reactions mediated by other members can catalyze the reactions necessary to proceed with synthesis and release the peptide from the complex. In addition, when this was performed with an engineered surfactin synthetase complex, we discovered that the complex could catalyze cyclization of the analog.

In certain embodiments, an engineered polypeptide comprises the C and A domains of the first module of SrfA-A, fused to a T domain which comprises a portion of the T domain of the first module of SrfA-A and a portion of the T domain of the second module of SrfA-B. In certain embodiments, the engineered polypeptide comprises a mutation that increases the yield of the lipopeptide relative to a polypeptide that does not have the mutation. In certain embodiments, the mutation is an engineered SrfA-A polypeptide with a P2051L mutation (numbered with respect to the amino acid sequence of native SrfA-A in GenBank® under Acc. no. NP_{—}388230, GI:16077417).

In certain embodiments, an engineered lipopeptide synthetase polypeptide comprises a deletion of an A domain and a T domain of the first peptide synthetase polypeptide, relative to the naturally occurring lipopeptide synthetase polypeptide. For example, the engineered polypeptide comprises an A domain and T domain of the second peptide synthetase domain, fused to a C domain which is a hybrid C domain comprising a portion of the C domain of the first peptide synthetase domain and a portion of the C domain of the second peptide synthetase domain. (e.g., wherein the polypeptide is produced by engineering a cell in which the DNA encoding homologous regions of the C domains of the first and second modules have been joined by deletion).

In certain embodiments, an engineered polypeptide comprises a C domain which includes a portion of the C domain of the first module of SrfA-A and a portion of the C domain of the second module of SrfA-B, fused to the A domain and T domain of the second module of SrfA-B.

Engineered Polypeptides Having Module Substitutions

We have found that one can engineer polypeptides to link modules that are not linked in naturally occurring synthetase polypeptides (e.g., modules from heterologous synthetases) to produce lipopeptides having a desired amino acid sequence. Accordingly, the present invention provides an engineered lipopeptide synthetase polypeptide that includes a first peptide synthetase domain of a first peptide synthetase polypeptide (e.g., a lipopeptide synthetase domain, e.g., a lipopeptide synthetase domain comprising a fatty acid linkage domain), and a second peptide synthetase domain of a second peptide synthetase polypeptide (e.g., a lipopeptide synthetase domain), wherein the first and second peptide synthetase domains are covalently linked such that the engineered lipopeptide synthetase polypeptide produces a lipopeptide comprising an amino acid encoded by the first peptide synthetase domain linked to an amino acid encoded by the second peptide synthetase domain.

In certain embodiments, the first peptide synthetase domain is the first module of SrfA-A, and the second peptide synthetase domain is a second module of a heterologous synthetase (e.g., tyrocidine synthetase, or gramicidin synthetase).

In certain embodiments, an engineered polypeptide further includes a third peptide synthetase domain. In certain embodiments, the first peptide synthetase domain is the first module of SrfA-A, the second peptide synthetase domain is a second module of a heterologous peptide synthetase (e.g., the second module of tyrocidine synthetase, or gramicidin synthetase) and the third peptide synthetase domain is the third module of SrfA-A.

Engineered Dipeptide and Oligopeptide Synthetases

We have also discovered that one can produce lipopeptides having two or more amino acids of a desired sequence by providing engineered lipopeptide synthetases in which a discrete set of peptide synthetase domains are linked to a thioesterases domain. Thus, the invention provides an engineered lipopeptide synthetase polypeptide that includes a first peptide synthetase domain of a naturally occurring lipopeptide synthetase polypeptide, and a second peptide synthetase domain of a naturally occurring lipopeptide synthetase polypeptide. The second peptide synthetase domain is covalently linked to a thioesterase domain of a peptide synthetase polypeptide.

In certain embodiments, the first peptide synthetase domain and the second peptide synthetase domain are domains from the same naturally occurring lipopeptide synthetase polypeptide. In certain embodiments, the first and second domains are from SrfA-A.

In certain embodiments, the first peptide synthetase domain and the thioesterase domain are from the same naturally occurring lipopeptide synthetase polypeptide complex. For example, the first peptide synthetase domain and thioesterase domains are from the SrfA complex. In certain embodiments, the engineered polypeptide includes a third peptide synthetase domain, upstream of (N-terminal to) the thioesterase domain, to provide a polypeptide that produces a tripeptide. One example of such a polypeptide includes modules 1, 2, and 3 of SrfA-A, linked to a thioesterase domain. Polypeptides can be engineered in this manner to produce longer lipopeptides as well (e.g., lipopeptides that are four, five, six, seven, eight, nine, ten, or more amino acids in length).

Those of ordinary skill in the art will be aware of a variety of naturally occurring polypeptides that comprise a naturally occurring peptide synthetase domain, fatty acid linkage domain, thioesterase domain and/or reductase domain that may advantageously be incorporated into an engineered polypeptide of the present invention. For example, any of a variety of naturally occurring peptide synthetase complexes (see section above entitled “Lipopeptide Synthetase Complexes”) may contain one or more of these domains, which domains may be incorporated into an engineered polypeptide of the present invention. Non-limiting examples of peptide synthetase complexes include surfactin synthetase, fengycin synthetase, arthrofactin synthetase, lichenysin synthetase, syringomycin synthetase, syringopeptin synthetase, saframycin synthetase, gramicidin synthetase, cyclosporin synthetase, tyrocidin synthetase, mycobacillin synthetase, polymyxin synthetase and bacitracin synthetase.

In certain embodiments, one or more such domains present in an engineered polypeptide of the present invention is not naturally occurring, but is itself an engineered domain. For example, an engineered domain present in an engineered polypeptide of the present invention may comprise one or more amino acid insertions, deletions, substitutions or transpositions as compared to a naturally occurring peptide synthetase domain, fatty acid linkage domain (e.g. a beta-hydroxy fatty acid linkage domain), thioesterase domain and/or reductase domain. In certain embodiments, an engineered peptide synthetase domain, fatty acid linkage domain (e.g. a beta-hydroxy fatty acid linkage domain), thioesterase domain and/or reductase domain present in an engineered polypeptide of the present invention comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or more amino acid insertions as compared to a naturally occurring domain. In certain embodiments, an engineered peptide synthetase domain, fatty acid linkage domain (e.g. a beta-hydroxy fatty acid linkage domain), thioesterase domain and/or reductase domain present in an engineered polypeptide of the present invention comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or more amino acid deletions as compared to a naturally occurring domain.

In certain embodiments, an engineered peptide synthetase domain, fatty acid linkage domain (e.g. a beta-hydroxy fatty acid linkage domain), thioesterase domain and/or reductase domain present in an engineered polypeptide of the present invention comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or more amino acid substitutions as compared to a naturally occurring domain. In certain embodiments, such amino acid substitutions result in an engineered domain that comprises an amino acid whose side chain contains a structurally similar side chain as compared to the amino acid in a naturally occurring peptide synthetase domain, fatty acid linkage domain, thioesterase domain and/or reductase domain. For example, amino acids with aliphatic side chains, including glycine, alanine, valine, leucine, and isoleucine, may be substituted for each other; amino acids having aliphatic-hydroxyl side chains, including serine and threonine, may be substituted for each other; amino acids having amide-containing side chains, including asparagine and glutamine, may be substituted for each other; amino acids having aromatic side chains, including phenylalanine, tyrosine, and tryptophan, may be substituted for each other; amino acids having basic side chains, including lysine, arginine, and histidine, may be substituted for each other; and amino acids having sulfur-containing side chains, including cysteine and methionine, may be substituted for each other.

In certain embodiments, amino acid substitutions result in an engineered domain that comprises an amino acid whose side chain exhibits similar chemical properties to an amino acid present in a naturally occurring peptide synthetase domain, fatty acid linkage domain (e.g. a beta-hydroxy fatty acid linkage domain), thioesterase domain and/or reductase domain. For example, in certain embodiments, amino acids that comprise hydrophobic side chains may be substituted for each other. In some embodiments, amino acids may be substituted for each other if their side chains are of similar molecular weight or bulk. For example, an amino acid in an engineered domain may be substituted for an amino acid present in the naturally occurring domain if its side chains exhibits a minimum/maximum molecular weight or takes up a minimum/maximum amount of space.

In certain embodiments, an engineered peptide synthetase domain, fatty acid linkage domain (e.g. a beta-hydroxy fatty acid linkage domain), thioesterase domain and/or reductase domain present in an engineered polypeptide of the present invention exhibits homology to a naturally occurring peptide synthetase domain, fatty acid linkage domain, thioesterase domain and/or reductase domain. In certain embodiments, an engineered domain of the present invention comprises a polypeptide or portion of a polypeptide whose amino acid sequence is 50, 55, 60, 65, 70, 75, 80, 85 or 90 percent identical or similar over a given length of the polypeptide or portion to a naturally occurring domain. In certain embodiments, an engineered domain of the present invention comprises a polypeptide or portion of a polypeptide whose amino acid sequence is 91, 92, 93, 94, 95, 96, 97, 98, or 99 percent identical or similar over a given length of the polypeptide or portion to a naturally occurring domain. The length of the polypeptide or portion over which an engineered domain of the present invention is similar or identical to a naturally occurring domain may be, for example, 5, 6, 7, 8, 9, 10, 15, 20, 25, 30, 35, 40, 45, 50, 60, 70, 80, 90, 100, 200, 300, 400, 500, 600, 700, 800, 900, 1000 or more amino acids.

In certain embodiments, an engineered peptide synthetase domain, fatty acid linkage domain (e.g. a beta-hydroxy fatty acid linkage domain), thioesterase domain and/or reductase domain present in an engineered polypeptide of the present invention comprises an amino acid sequence that conforms to a consensus sequence of a class of engineered peptide synthetase domains, fatty acid linkage domains, thioesterase domains and/or reductase domains. For example, a thioesterase domain may comprise the consensus sequence: [LIV]-{KG}-[LIVFY]-[LIVMST]-G-[HYWV]-S-{YAG}-G-[GSTAC], and a reductase domain may comprise the consensus sequence: [LIVSPADNK]-x(9)-{P}-x(2)-Y-[PSTAGNCV]-[STAGNQCIVM]-[STAGC]-K-{PC}-[SAGFYR]-[LIVMSTAGD]-x-{K}-[LIVMFYW]-{D}-x-{YR}-[LIVMFYWGAPTHQ]-[GSACQRHM] (SEQ ID NO:______).

In certain embodiments, an engineered peptide synthetase domain, fatty acid linkage domain (e.g. a beta-hydroxy fatty acid linkage domain), thioesterase domain and/or reductase domain present in an engineered polypeptide of the present invention is both: 1) homologous to a naturally occurring engineered peptide synthetase domain, fatty acid linkage domain, thioesterase domain and/or reductase domain of the present invention, and 2) comprises an amino acid sequence that conforms to a consensus sequence of a class of engineered peptide synthetase domain, fatty acid linkage domain, thioesterase domain and/or reductase domains.

In certain embodiments, engineered polypeptides of the present invention comprise two or more naturally occurring polypeptide domains that are covalently linked (directly or indirectly) in the polypeptide in which they occur, but are linked in the engineered polypeptide in a non-natural manner. As a non-limiting example, two naturally occurring polypeptide domains that are directly covalently linked may be separated in the engineered polypeptide by one or more intervening amino acid residues. Additionally or alternatively, two naturally occurring polypeptide domains that are indirectly covalently linked may be directly covalently linked in the engineered polypeptide, e.g. by removing one or more intervening amino acid residues. As a non-limiting example, engineered polypeptides of the present invention may comprise a peptide synthetase domain and beta-hydroxy fatty acid linkage domain from the SRFA protein, and a thioesterase domain from the SrfC protein, which peptide synthetase domain, beta-hydroxy fatty acid linkage domain and thioesterase domain are covalently linked to each other (e.g. via peptide bonds).

In certain embodiments, two naturally occurring peptide domains that are from different peptide synthetases are covalently joined to generate an engineered polypeptide of the present invention. As a non-limiting example, engineered polypeptides of the present invention may comprise a peptide synthetase domain and beta-hydroxy fatty acid linkage domain from the SRFA protein, and a peptide synthetase domain from a heterologous peptide synthetase (e.g., tyrocidine synthetase, or gramicidin synthetase), which peptide synthetase domains are covalently linked to each other (e.g. via peptide bonds). In certain embodiments, an engineered polypeptide comprises a peptide synthetase domain and beta-hydroxy fatty acid linkage domain from the SRFA protein (e.g., module 1 of SrfA-A), linked to a second peptide synthetase domain from a heterologous peptide synthetase, linked to a third peptide synthetase domain from the SRFA protein (e.g., linked to module 3 of SrfA-A).

The present invention encompasses engineered polypeptides comprised of these and other peptide synthetase domains from a variety of peptide synthetase complexes. In certain embodiments, engineered polypeptides of the present invention comprise at least one naturally occurring polypeptide domain and at least one engineered domain. In certain embodiments, engineered polypeptides of the present invention comprise one or more additional peptide synthetase domains, fatty acid linkage domains, thioesterase domains and/or reductase domains, and still produce a lipopeptide of interest. Thus, the present invention encompasses the recognition that engineered polypeptides comprising additional peptide synthetase domains, fatty acid linkage domains, thioesterase domains and/or reductase domains beyond those that are minimally required to produce an lipopeptide of interest may be advantageous in producing such lipoeptides.

### Lipopeptides

A variety of lipopeptides may be generated by compositions and methods of the present invention. By employing specific peptide synthetase domains in engineered polypeptides, one skilled in the art will be able to generate a specific lipopeptide following the teachings of the present invention.

The present invention provides lipopeptides that are analogs of naturally occurring lipopeptides. In some embodiments, an analog lipopeptide includes a deletion of an amino acid, relative to the naturally occurring lipopeptide (e.g., a deletion of the first amino acid or second amino acid in the naturally occurring lipopeptide). In some embodiments, an analog lipopeptide includes a substitution of an amino acid relative to the naturally occurring lipopeptide (e.g., a substitution of the second or third amino acid). Such lipopeptides can be produced by engineering lipopeptide synthetases as described herein.

In certain embodiments, a lipopeptide generated by an engineered lipopeptide synthetase described herein has the following amino acid sequence: L-Glu-D-Leu-L-Val-L-Asp-D-Leu-L-Leu (SEQ ID NO:______), wherein the lipopeptide comprises a fatty acid moiety on the L-Glu residue. (Herein, “L” and “D” before a three letter abbreviation for an amino acid, refer to L and D isomers of the amino acid). In certain embodiments, the lipopeptide is cyclic.

In some embodiments, the lipopeptide has the following amino acid sequence: L-Glu-X-D-Leu-L-Val-L-Asp-D-Leu-L-Leu (SEQ ID NO:______), wherein X is any amino acid, and wherein the lipopeptide comprises a fatty acid moiety on the L-Glu residue. In certain embodiments, X is L-Tyr. In some embodiments, the lipopeptide is cyclic.

In certain embodiments, the lipopeptide has the following amino acid sequence: L-Leu-D-Leu-L-Val-L-Asp-D-Leu-L-Leu (SEQ ID NO:______), wherein the lipopeptide comprises a fatty acid moiety on the L-Leu residue. In certain embodiments, the lipopeptide is cyclic.

In certain embodiments, lipopeptides generated by compositions and methods of the present invention comprise an amino acid selected from one of the twenty amino acids commonly employed in ribosomal peptide synthesis. Thus, lipopeptides of the present invention may comprise alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and/or valine. In certain embodiments, lipopeptides of the present invention comprise amino acids other than these twenty. For example, lipopeptides of the present invention may comprise amino acids used less commonly during ribosomal polypeptide synthesis such as, without limitation, selenocysteine and/or pyrrolysine. In certain embodiments, lipopeptides of the present invention comprise amino acids that are not used during ribosomal polypeptide synthesis such as, without limitation, norleucine, beta-alanine and/or ornithine, and/or D-amino acids.

In certain embodiments, lipopeptides produced by engineered polypeptides as described herein can have 2, 3, 4, 5, 6, 7, 8, 9, 10, 11, 12, 13, 14, 15, or more amino acid residues. By way of example, a lipopeptide produced by an engineered lipopeptide synthetase which is a deletion mutant produces a lipopeptide having one less amino acid than a naturally occurring form of the lipopeptide. In one example, a surfactin analog produced by an engineered surfactin synthetase polypeptide has 6 residues, whereas natural surfactin has 7 residues. In another example, a syringomycin analog produced by an engineered syringomycin synthetase polypeptide has 8 residues, whereas natural syringomycin has 9 residues. In certain embodiments, analog lipopeptides produced by the engineered polypeptides described herein have improved characteristics (e.g., relative to a naturally occurring form of the lipopeptide). In certain embodiments, a lipopeptide produced by an engineered synthetase polypeptide has similar or improved solubility, similar or increased cytotoxicity to a pest or pathogen (e.g., a plant pest or fungus), similar or decreased cytotoxicity to a host cell (e.g., plant cell), similar or more potent surfactant properties, similar or enhanced nutritional value as a food or feed additive, or similar or enhanced efficacy as a cosmetic additive.

Assays for evaluating characteristics of lipopeptides are known in the art. For example, surface-active properties of a lipopeptide preparation can be measured by the drop weight method (Harkins and Brown, J. Am. Chem. Soc. 41:499-523, 1919; Hutchinson et al., Mol. J. Plant. Path. Int. 8:610-620, 1995). Pore-forming activity of lipopeptides can be evaluated using an artificial bilayer conductance assay (Hutchinson et al., Mol. Plant Micr. Int. 10(3):347-354, 1997). Hemolytic activity can be measured by detecting erythrocyte lysis in the presence of the lipopeptide (Hutchinson et al., Mol. J. Plant. Path. Int. 8:610-620, 1995).

As will be understood by those of ordinary skill in the art after reading this specification, it will typically be the peptide synthetase domain of engineered polypeptides of the present invention that specify the identity of the amino acids of lipopeptides. For example, the first peptide synthetase domain of the SRFA protein of the surfactin synthetase complex recognizes and specifies glutamic acid, the first amino acid in surfactin. Thus, in certain embodiments, engineered polypeptides of the present invention comprise the first peptide synthetase domain of the SRFA protein of the surfactin synthetase complex, such that the lipopeptide produced by the engineered polypeptide comprises glutamic acid. The present invention encompasses the recognition that engineered polypeptides of the present invention may comprise other peptide synthetase domains from the surfactin synthetase complex and/or other peptide synthetase complexes in order to generate lipopeptides including other amino acids.

In certain embodiments, engineered polypeptides of the present invention comprise an engineered peptide synthetase domain that is similar to a naturally occurring peptide synthetase domain. For example, such engineered peptide synthetase domains may comprise one or more amino acid insertions, deletions, substitutions, or transpositions as compared to a naturally occurring peptide synthetase domain. Additionally or alternatively, such engineered peptide synthetase domains may exhibit homology to a naturally occurring peptide synthetase domain, as measured by, for example, percent identity or similarity at the amino acid level. Additionally or alternatively, such engineered peptide synthetase domains may comprise one or more amino acid sequences that conform to a consensus sequence characteristic of a given naturally occurring peptide synthetase domain. In certain embodiments, an engineered peptide synthetase domain that is similar to a naturally occurring peptide synthetase domain retains the amino acid specificity of the naturally occurring peptide synthetase domain. For example, the present invention encompasses the recognition that one or more amino acid changes may be made to the first peptide synthetase domain of the SRFA protein of the surfactin synthetase complex, such that the engineered peptide synthetase domain still retains specificity for glutamic acid.

Such engineered peptide synthetase domains may exhibit one or more advantageous properties as compared to a naturally occurring peptide synthetase domain. For example, engineered polypeptides comprising such engineered peptide synthetase domains may yield an increased amount of the lipopeptide, may be more stable in a given host cell, may be less toxic to a given host cell, etc. Those of ordinary skill in the art will understand various advantages of engineered peptide synthetase domains of the present invention, and will be able to recognize and optimize such advantages in accordance with the teachings herein.

In certain embodiments, lipopeptides generated by compositions and methods of the present invention comprise a fatty acid moiety. A fatty acid of acyl amino acids of the present invention may be any of a variety of fatty acids known to those of ordinary skill in the art. For example, lipopeptides of the present invention may comprise saturated fatty acids such as, without limitation, butryic acid, caproic acid, caprylic acid, capric acid, lauric acid, myristic acid, palmitic acid, stearic arachidic acid, behenic acid, and/or lignoceric acid. In certain embodiments, lipopeptides of the present invention may comprise unsaturated fatty acids such as, without limitation, myristoleic acid, palmitoleic acid, oliec acid, linoleic acid, alpha-linolenic acid, arachidonic acid, eicosapentaenoic acid, erucic acid, and/or docosahexaenoic acid. Other saturated and unsaturated fatty acids that may be used in accordance with the present invention will be known to those of ordinary skill in the art. In certain embodiments, lipopeptides produced by compositions and methods of the present invention comprise beta-hydroxy fatty acids as the fatty acid moiety. As is understood by those of ordinary skill in the art, beta-hydroxy fatty acids comprise a hydroxy group attached to the third carbon of the fatty acid chain, the first carbon being the carbon of the carboxylate group.

As will be understood by those of ordinary skill in the art after reading this specification, it will typically be the fatty acid linkage domain of engineered polypeptides of the present invention that specify the identity of the fatty acid of the acyl amino acid. For example, the beta-hydroxy fatty acid linkage domain of the SRFA protein of the surfactin synthetase complex recognizes and specifies beta-hydroxy myristic acid, the fatty acid present in surfactin. Thus, in certain embodiments, engineered polypeptides of the present invention comprise the beta-hydroxy fatty acid linkage domain of the SRFA protein of the surfactin synthetase complex, such that the lipopeptide produced by the engineered polypeptide comprises beta-hydroxy myristic acid. The present invention encompasses the recognition that engineered polypeptides of the present invention may comprise other beta-hydroxy fatty acid linkage domains from other peptide synthetase complexes in order to generate other lipopeptides.

In certain embodiments, engineered polypeptides of the present invention comprise an engineered fatty acid linkage domain (e.g. a beta-hydroxy fatty acid linkage domain) that is similar to a naturally occurring fatty acid linkage domain. For example, such engineered fatty acid linkage domains may comprise one or more amino acid insertions, deletions, substitutions, or transpositions as compared to a naturally occurring fatty acid linkage domain. Additionally or alternatively, such engineered fatty acid linkage domains may exhibit homology to a naturally occurring fatty acid linkage domain, as measured by, for example, percent identity or similarity at the amino acid level. Additionally or alternatively, such engineered fatty acid linkage domains may comprise one or more amino acid sequences that conform to a consensus sequence characteristic of a given naturally occurring fatty acid linkage domain. In certain embodiments, an engineered fatty acid linkage domain that is similar to a naturally occurring fatty acid linkage domain retains the fatty acid specificity of the naturally occurring fatty acid linkage domain. For example, the present invention encompasses the recognition that one or more amino acid changes may be made to the beta-hydroxy fatty acid linkage domain of the SRFA protein of the surfactin synthetase complex, such that the engineered beta-hydroxy fatty acid linkage domain still retains specificity for beta-hydroxy myristic acid. As will be recognized by those of ordinary skill in the art after reading this specification, engineered polypeptides containing such an engineered beta-hydroxy fatty acid linkage domain will be useful in the generation of lipopeptides comprising beta-hydroxy myristic acid.

Engineered fatty acid linkage domains may exhibit one or more advantageous properties as compared to a naturally occurring fatty acid linkage domain. For example, engineered polypeptides comprising such engineered fatty acid linkage domains may yield an increased amount of the lipopeptide, may be more stable in a given host cell, may be less toxic to a given host cell, etc. Those of ordinary skill in the art will understand various advantages of engineered fatty acid linkage domains of the present invention, and will be able to recognize and optimize such advantages in accordance with the teachings herein.

In certain embodiments, engineered polypeptides of the present invention comprise an engineered thioesterase or reductase domain that is similar to a naturally occurring thioesterase or reductase domain. For example, such engineered thioesterase or reductase domains may comprise one or more amino acid insertions, deletions, substitutions, or transpositions as compared to a naturally occurring thioesterase or reductase domain. Additionally or alternatively, such engineered thioesterase or reductase domains may exhibit homology to a naturally occurring thioesterase or reductase domain, as measured by, for example, percent identity or similarity at the amino acid level. Additionally or alternatively, such engineered thioesterase or reductase domains may comprise one or more amino acid sequences that conform to a consensus sequence characteristic of a given naturally occurring thioesterase or reductase domain. In certain embodiments, an engineered thioesterase or reductase domain that is similar to a naturally occurring thioesterase or reductase domain retains the ability of the naturally occurring thioesterase or reductase domain to release a lipopeptide from the engineered polypeptide that produces it.

Engineered thioesterase or reductase domains may exhibit one or more advantageous properties as compared to a naturally occurring thioesterase or reductase domain. For example, engineered polypeptides comprising such engineered thioesterase or reductase domains may yield an increased amount of the lipopeptide, may be more stable in a given host cell, may be less toxic to a given host cell, etc. Those of ordinary skill in the art will understand various advantages of engineered thioesterase or reductase domains of the present invention, and will be able to recognize and optimize such advantages in accordance with the teachings herein.

In certain embodiments, compositions and methods of the present invention are useful in large-scale production of lipopeptides. In certain embodiments, lipopeptides are produced in commercially viable quantities using compositions and methods of the present invention. For example, engineered polypeptides of the present invention may be used to produce lipopeptides to a level of at least 5, 6, 7, 8, 9, 10, 11, 12, 13, 14, 15, 16, 17, 18, 19, 20, 30, 40, 50, 60, 70, 80, 90, 100, 110, 120, 130, 140, 150, 150, 200, 250, 300, 400, 500, 600, 700, 800, 900, 1000 mg/L or higher. As will be appreciated by those skilled in the art, biological production of lipopeptides using engineered polypeptides of the present invention achieves certain advantages over other methods of producing lipopeptides. For example, as compared to chemical production methods, production of lipopeptides using compositions and methods of the present invention reduces the necessity of using harsh and sometimes dangerous chemical reagents in the manufacturing process, reduces the difficulty and efficiency of the synthesis itself by utilizing host cells as bioreactors, and reduces the fiscal and environmental cost of disposing of chemical by-products. Other advantages will be clear to practitioners who utilize compositions and methods of the present invention.

### Host Cells

Engineered polypeptides of the present invention may be introduced in any of a variety of host cells for the production of lipopeptides. As will be understood by those skilled in the art, engineered polypeptides will typically be introduced into a host cell in an expression vector. So long as a host cell is capable of receiving and propagating such an expression vector, and is capable of expressing the engineered polypeptide, such a host cell is encompassed by the present invention. An engineered polypeptide of the present invention may be transiently or stably introduced into a host cell of interest. For example, an engineered polypeptide of the present invention may be stably introduced by integrating the engineered polypeptide into the chromosome of the host cell. Additionally or alternatively, an engineered polypeptide of the present invention may be transiently introduced by introducing a vector comprising the engineered polypeptide into a host cell, which vector is not integrated into the genome of the host cell, but is nevertheless propagated by the host cell. In certain embodiments, a cell is manipulated to delete a genomic region encoding a portion of a naturally occurring lipopeptide synthetase polypeptide, thereby producing a cell that expresses an engineered lipopeptide synthetase polypeptide which is a deletion mutant. Examples of such cells and engineered polypeptides are described below, e.g., in the Examples.

In certain embodiments, a host cell is a bacterium. Non-limiting examples of bacteria that are useful as host cells of the present invention include bacteria of the genera Escherichia, Streptococcus, Bacillus, and a variety of other genera known to those skilled in the art. In certain embodiments, an engineered polypeptide of the present invention is expressed in a host cell of the species Bacillus subtilis.

Bacterial host cells of the present invention may be wild type. Alternatively, bacterial host cells of the present invention may comprise one or more genetic changes as compared to wild type species. In certain embodiments, such genetic changes are beneficial to the production of lipopeptides in the bacterial host. For example, such genetic changes may result in increased yield or purity of the lipopeptides, and/or may endow the bacterial host cell with various advantages useful in the production of acyl amino acids (e.g., increased viability, ability to utilize alternative energy sources, etc.).

In certain embodiments, the host cell is a plant cell. Those skilled in the art are aware of standard techniques for introducing an engineered polypeptide of the present invention into a plant cell of interest such as, without limitation, gold bombardment and agrobacterium transformation. In certain embodiments, the present invention provides a transgenic plant that comprises an engineered polypeptide that produces a lipopeptide of interest. Any of a variety of plants species may be made transgenic by introduction of an engineered polypeptide of the present invention, such that the engineered polypeptide is expressed in the plant and produces a lipopeptide of interest. The engineered polypeptide of transgenic plants of the present invention may be expressed systemically (e.g. in each tissue at all times) or only in localized tissues and/or during certain periods of time. Those skilled in the art will be aware of various promoters, enhancers, etc. that may be employed to control when and where an engineered polypeptide is expressed.

In certain embodiments, an engineered lipopeptide synthetase expressed in a plant utilizes fatty acids naturally present in the plant cell, although such fatty acids may differ in composition (e.g., carbon chain length) than natural fatty acid substrates of the fatty acid linkage domain of the synthetase. In some embodiments, the engineered polypeptide to be expressed in a plant cell is selected so as to be compatible with the fatty acids produced by the plant cell. For example, corn produces fatty acids having a length of 16 carbons, such as palmitic acid (16:0), and palmitoletic acid (16:1). One can select for expression in corn cells an engineered lipopeptide synthetase having a fatty acid linkage domain that attaches fatty acids having 16 to 17 carbons, e.g., as produced by mycosubtilin and bacillomycin F synthetases.

### Applications

Insects, including insects that are threats to agriculture crops, produce acyl amino acids and lipopeptides that are likely to be important or essential for insect physiology. For example, an enzyme related to peptide synthetases produces the product of the Drosophila Ebony genes, which product is important for proper pigmentation of the fly, but is also important for proper function of the nervous system (see e.g., Richardt et al., Ebony, a novel nonribosomal peptide synthetase for beta-alanine conjugation with biogenic amines in Drosophila, J. Biol. Chem., 278(42):41160-6, 2003). Acyl amino acids are also produced by certain Lepidoptera species that are a threat to crops. Thus, compositions and methods of the present invention may be used to produce transgenic plants that produce a lipopeptide of interest that interferes with the function acyl amino acids and lipopeptides produced by the insects. In some embodiments, lipopeptides of interest are applied to plants (e.g., leaves, roots, or soil around the roots). Lipopeptide-containing compositions can be applied as wettable powders, granules, or as part of a liquid formulation (see, e.g., U.S. Pat. No. 6,638,910). In some embodiments, bacterial host cells (e.g., live Bacillus) that express one or more lipopeptides of interest are applied to plants to provide insecticidal activitiy.

Compositions and methods of the present invention may be used to kill such insects or otherwise disrupt their adverse effects on crops. For example, an engineered polypeptide that produces a lipopeptide that is toxic to a given insect species may be introduced into a plant such that insects that infest such a plant are killed. Additionally or alternatively, an engineered polypeptide that produces a lipopeptide that disrupts an essential activity of the insect (e.g., feeding, mating, etc.) may be introduced into a plant such that the commercially adverse effects of insect infestation are inhibited. In certain embodiments, a lipopeptide of the present invention that mitigates an insect's adverse effects on a plant is a lipopeptide that is naturally produced by such an insect. In certain embodiments, a lipopeptide of the present invention that mitigates an insect's adverse effects on a plant is a structural analog of a lipopeptide that is naturally produced by such an insect. Compositions and methods of the present invention are extremely powerful in allowing the construction of engineered polypeptides that produce any of a variety of lipopeptides, which lipopeptides can be used in controlling or eliminating harmful insect infestation of one or more plant species.

Lipopeptides (e.g., novel lipopeptides produced by the methods described herein) can be evaluated for phytotoxicity, to permit selection of lipopeptides that are less toxic to cells. Assays for measuring phytotoxicity are known in the art. In one exemplary assay, lipopeptide phytotoxicity is evaluated in assays that employ plant protoplasts, as described in Hutchinson and Gross, Mol. Plant Micr. Int. 10(3):347-354, 1997. In these assays, protoplasts are prepared from tobacco leaves and incubated with lipopeptide preparations at a range of concentrations. Protoplast lysis and/or the rate of cytoplasmic influx of ⁴⁵Ca²⁺ into the protoplasts is determined.

In addition to insecticidal properties, many lipopeptides have potent activity towards microbial pathogens, e.g., bacteria, and fungi. Lipopeptide compositions can be employed in methods of inhibiting infection by these types of pathogens in plants, just as lipopeptides may be used to prevent adverse effects of insect infestation as described above. Insecticidal, bactericidal, and fungicidal properties of lipopeptide compositions can be evaluated by any number of methods known in the art. In some embodiments, insecticidal activity of a lipopeptide composition is determined by preparing agar plates onto which the lipopeptide composition is applied. Test organisms are placed on the plates and incubated for a period of time, after which survival of the organisms is determined (see, e.g., U.S. Pat. No. 6,638,910). This type of method is suitable for testing survival of organisms such as pre-adult corn rootworms (Diabrotica undecimpunctata), pre-adult German cockroaches (Blatella germanica), pre-adult beet armyworms (Spodoptera exigua), pre-adult flies (Drosophila melanogaster), or the nematode Caenorhabditis elegans. In some embodiments, insecticidal activity is tested by applying a liquid or powder lipopeptide composition to a plant that is infested with a pathogen or pest of interest, and monitoring the infestation after application of the composition. Additional methods for testing insecticidal activity toward aphids, bacteria and fungal pathogens of plant species are described in U.S. Pat. No. 6,638,910.

### Examples

### Example 1

### Engineering a Lipopeptide Synthetase Polypeptide with a Deletion of a Thioloation Domain and a Condensation Domain

We engineered a recombinant lipopeptide synthetase polypeptide in which the highly homologous sequences in the adenylation domains of modules 2 and 3 of the first surfactin synthetase, SrfA-A, were joined. The goal of this experiment was to provide a synthetase that would produce a 6-member surfactin ring lacking the second amino acid of surfactin, L-leucine. A DNA construct was produced which contains at its ends homologous sequences (upstream and downstream) to those present in the Bacillus subtilis chromosome. In between these sequences, which establish the insertion location of the construct, there are 78 bp direct repeats (DR) that flank an “upp-kanamycin” cassette. The excision of the cassette through recombination of the DR leaves solely the desired mutation in the chromosome.

Due to the high similarity that exists among modules, it was advantageous to perform nested PCR reactions to amplify genomic DNA sequences. The upstream flanking sequence was amplified from genomic DNA of OKB105 cells using primers:

(SEQ ID NO: )

1C: 5′-ATGGTGATGCTTTCCGCTTACTATACG-3′,
and

(SEQ ID NO: )

1D: 5′-CGTTCCGGAAGTATACGTCAGGTTGGC-3′.

This DNA fragment was subsequently amplified with primers: TAIL-5′-1CD-FW-MOD: 5′-CTGCGATCAGTGTTCCCACTmAATGGTGATGCTTTCCGCTTACTATACG-3′ (SEQ ID NO:______), and TAIL-3′-1CD-BK-MOD: 5′-CTCTGGACTGTCGAAAGCAAmGCGTTCCGGAAGTATACGTCAGGTTGGC-3′ (SEQ ID NO:______).

This fragment was annealed to a PCR product derived from amplifying pUC19 with primers: pUC19 sense-2: 5′-CTTGCTTTCGACAGTCCAGAmGGCCAGTGAATTCGAGCTCGGTACC-3′ (SEQ ID NO:______), and pUC19 anti-2: 5′-TAGTGGGAACACTGATCGCAmGACCCAACTTAATCGCCTTGCAGCACATC-3′ (SEQ ID NO:______).

The annealed fragments were transformed into Sure cells and selected on CG-Amp. The resulting plasmid was named pUC19-1CD.

Direct repeats of 48, 78, and 102 bp were obtained by using pUC19-1CD as template. The 48 bp-fragment was obtained using primers 1-DR-cloning-BK-MOD: 5′-AACACCCTTTGGCTGACCTGmUCGTTCCGGAAGTATACGTCAGGTTGGC-3′ (SEQ ID NO:______), and 1-DR-FW-48 bp-MOD: 5′-CTAATGAGTGAGCTAATCTCmUCCTCTTGATTCTGCAGCAATGGCCAAC-3′ (SEQ ID NO:______).

The 78 bp-fragment was obtained using primers 1-DR-cloning-BK-MOD: 5′-AACACCCTTTGGCTGACCTGmUCGTTCCGGAAGTATACGTCAGGTTGGC-3′ (SEQ ID NO:______), and 1-DR-FW-78 bp-MOD: 5′-CTAATGAGTGAGCTAATCTCmUTATCATGCCGATGCGCGAAATCTCG-3′(SEQ ID NO:______).

The 102 bp-fragment was obtained using primers 1-DR-cloning-BK-MOD: 5′-AACACCCTTTGGCTGACCTGmUCGTTCCGGAAGTATACGTCAGGTTGGC-3′ (SEQ ID NO:______), and 1-DR-FW-102 bp-MOD: 5′-CTAATGAGTGAGCTAATCTCmUGTGCTAGCCGATGAGGAAGAAAG-3′ (SEQ ID NO:______).

These three fragments were cloned into pUC19-1CD that was opened using PUC19-anti-3-MOD: 5′-AGAGATTAGCTCACTCATTAmGGCACCCCAGGCTTTACACTTTATGCTTC-3′ (SEQ ID NO:______), and 1-pUC19-4-DR-sense-3-MOD:5′-ACAGGTCAGCCAAAGGGTGTmUCCAGCTGCATTAATGAATCGGCCAAC-3′(SEQ ID NO:______).

The annealed fragments were transformed into Sure cells and selected on CG-Amp. The resulting plasmids were named pUC19-1CD-48 bp, pUC19-1CD-78 bp, pUC19-1CD-102 bp.

The downstream flanking sequence was amplified from genomic DNA of OKB105 cells using primers: 1-G:5′-GACCTCTGTTGTATTGAATGACGTCTTCCTG-3′ (SEQ ID NO:______), and 1-H:5′-ACCGGACAGCCGAAGGGTGTCATGGTCGAGC-3′ (SEQ ID NO:______).

This DNA fragment was subsequently amplified with primers:1-DR-HG-FW:5′-ATGGTCGAGCATCATGCGCTmUGTGAACCTTTGCTTCTGGCACCACGAC-3′(SEQ ID NO:______), and 1-HG-4-vect+DR-BK:5′-GAGTGCAGAATACTCAAACCmGGACCTCTGTTGTATTGAATGACGTCTTCC-3′(SEQ ID NO:______).

This fragment was annealed separately to each of the PCR products derived from amplifying pUC19-1CD-48 bp, pUC19-1CD-78 bp, pUC19-1CD-102 bp with 1-DR-in-Vect-4-HG-BK:5′-AAGCGCATGATGCTCGACCAmUAACACCCTTTGGCTGACCTGTCGTTC-3′ (SEQ ID NO:______), and Vector-FW-4-HG-1&2: 5′-CGGTTTGAGTATTCTGCACTmCTTCCGCTTCCTCGCTCACTGACTC-3′ (SEQ ID NO:______).

The annealed fragments were transformed into Sure cells and selected on CG-Amp. The resulting plasmids were named pUC19-1CD-48DR-1HG, pUC19-1CD-78DR-1HG, pUC19-1CD-102DR-1HG.

We observed that when trying to identify plasmids pUC19-1CD-48 bp, pUC19-1CD-78 bp, pUC19-1CD-102 bp, pUC19-1CD-48DR-1HG, pUC19-1CD-78DR-1HG, pUC19-1CD-102DR-1HG, some candidate colonies underwent recombination in E. coli between the 48 bp, 78 bbp, and 102 bp direct repeats. Nonetheless, we were able to identify the desired sequences with ease.

However, when we tried to insert the upp-kan cassette in between the 1CD sequence and the DR, we were unable to construct such plasmid. Therefore, we decided to join the upp-kan cassette using restriction enzymes and T4 DNA ligase to engineer three constructs.

The upstream flanking sequence was obtained using the primers 1CD-and-2CD-FW: 5′-GGATGTGCTGCAAGGCGATTAAGTTGGGTCTG-3′ (SEQ ID NO:______), and 1CD-BstXI-BK: 5′-ATGCTAATCCACTCTCTTGGCGTTCCGGAAGTATACGTCAGGTTGGC-3′ (SEQ ID NO:______).

The upp-kan cassette was obtained by amplifying from pUC-UPP-KAN using the primers UPP-KAN-BstXI-FW: 5′-ATGCTAAGCCAAGAGAGTGGGTTTTTTGACGATGTTCTTGAAACTCAATG-3′ (SEQ ID NO:______), and UPP-KAN-and-KAN-BglI-BK: 5′-ATATCTGAGCCAGAGAGGCACCAATCAAAAAACAGATGGCCGCTATTAAAGC-3′. (SEQ ID NO:______).

The downstream sequence was obtained from pUC19-1CD-48DR-1HG, pUC19-1CD-78DR-1HG, pUC19-1CD-102DR-1HG using primers 1HG-and-2HG-BglI-FW: 5′-ATTACTACGCCTCTCTGGCACACAACATACGAGCCGGAAGCATAAAGTG-3′ (SEQ ID NO:______), and 1HG-and-2HG-BK: 5′-TGATTCTGTGGATAACCGTATTACCGCCTTTGAGTG-3′ (SEQ ID NO:______).

The upstream flanking sequence and the UPP-KAN fragment were digested with BstXI and ligated with T4 DNA ligase. The ligation reaction was then used as a template to re-amplify the ligated product using 1CD-and-2CD-FW and UPP-KAN-and-KAN-BglI-BK.

The resulting PCR product as well as the downstream flanking sequence were digested with BglI and ligated. The ligation mixtures 1CD-UPP-KAN-48DR-1HG, 1CD-UPP-KAN-78DR-1HG, and 1CD-UPP-KAN-102DR-1HG were cleaned using Qiagen's PCR purification kit and transformed into competent OKB105 Δupp Spect^{R} cells and plated on LB containing 30 μg/ml kanamycin plates. We obtained colonies from the ligation mixture that contained a 78 bp direct repeat. We did not obtain colonies from the ligation mixtures that contained 48 bp or 102 bp repeats. We observed an improved efficiency in obtaining the desired constructs when the direct repeat was 78 bp, as compared to efficiency when the direct repeat was 45 bp, as described below in Example 2.

In this experiment, 75 out 80 colonies picked had the desired recombination event. After sequencing, 10 out 10 from the 75 clones sequenced had the desired sequence. We were unable to detect the expected surfactin analog, as judged by mass spectrometry analysis.

### Example 2

### Engineering a Lipopeptide Synthetase Polypeptide with a Deletion of a Condensation Domain and an Adenylation Domain

We engineered a recombinant lipopeptide synthetase polypeptide in which the highly homologous sequences in the thiolation domains of modules 1 and 2 of the first surfactin synthetase, SrfA-A, were joined, with the goal of producing a 6-member surfactin ring lacking the amino acid L-leucine. To produce the recombinant polypeptide, we engineered a DNA construct, which contains at its ends homologous sequences (upstream and downstream) to those present in the Bacillus subtilis chromosome. The table below shows an alignment of the homologous region within the thiolation domains of modules 1, 2, and 3 of surfactin synthetase.

TABLE 2
Alignment of homologous regions
in thiolation domains of SrfA modules

| 984 in mod one and 3052 in mod 3. | |
|---|---|
| | \| --------- |
| mod one: | WQDVLNV--EKAGIFDNFFETGGHSLKA |
| mod two: | WAQVLQA--EQVGAYDHFFDIGGHSLAGMK |
| mod three: | WQDVLGM--SEVGVTDNFFSLGGDSIKGI |
| pfam ref: | ..WAEVLGVDPDEIGIDDNFFELGGDAVLE.... |

In between these sequences, which establish the insertion location of the construct, there are 45 bp direct repeats that flank an “upp-kanamycin” cassette. The excision of the cassette through recombination of the DR leaves solely the desired mutation in the chromosome. Constructs with 78 and 102 bp repeats were also constructed, but they failed to produce colonies when DNA was transformed into Bacillus.

Due to the high similarity that exists among modules, it was advantageous to do nested PCR reactions to amplify genomic DNA sequences. The upstream flanking sequence was amplified from genomic DNA of OKB105 cells using primers: 2C-MOD:5′-CATCATTTGGTTGAATCTCTGCAGCAGACG-3′ (SEQ ID NO:______), and 2D: 5′-GTCAAAGATCCCCGCCTTCTCAACG-3′ (SEQ ID NO:______).

This DNA fragment was subsequently amplified with primers: TAIL-5′-2CD-FW-MOD: 5′-CTGCGATCAGTGTTCCCACTmACATCATTTGGTTGAATCTCTGCAGCAGAC-3′ (SEQ ID NO:______), and TAIL-3′-2CD-BK-MOD: 5′-CTCTGGACTGTCGAAAGCAAmGGTCAAAGATCCCCGCCTTCTCAACG-3′ (SEQ ID NO:______).

This fragment was annealed to a per product derived from amplifying pUC19 with primers: pUC19 sense-2: 5′-CTTGCTTTCGACAGTCCAGAmGGCCAGTGAATTCGAGCTCGGTACC-3′ (SEQ ID NO:______), and pUC19 anti-2: 5′-TAGTGGGAACACTGATCGCAmGACCCAACTTAATCGCCTTGCAGCACATC-3′ (SEQ ID NO:______).

The annealed fragments were transformed into Sure cells and selected on CG-Amp. The resulting plasmid was named pUC19-2CD.

Direct repeats of 45, 78, and 102 bp were obtained by using pUC19-2CD as template. The 45 bp-fragment was obtained using primers 2-DR-cloning-BK-MOD: 5′-TCCTCCAATGTCAAAGAAGTmGGTCAAAGATCCCCGCCTTCTCAACG-3′ (SEQ ID NO:______), and 2-DR-FW-45 bp-MOD: 5′-CTAATGAGTGAGCTAATCTCmUATTTGGCAGGACGTGCTGAACGTTGAG-3′ (SEQ ID NO:______).

The 78 bp-fragment was obtained using primers 2-DR-cloning-BK-MOD: 5′-TCCTCCAATGTCAAAGAAGTmGGTCAAAGATCCCCGCCTTCTCAACG-3′ (SEQ ID NO:______), and 2-DR-FW-78 bp-MOD: 5′-CTAATGAGTGAGCTAATCTCmUCCGCGAAATGAGACTGAAAAAGCAATCG-3′ (SEQ ID NO:______).

The 102 bp-fragment was obtained using primers 2-DR-cloning-BK-MOD: 5′-TCCTCCAATGTCAAAGAAGTmGGTCAAAGATCCCCGCCTTCTCAACG-3′ (SEQ ID NO:______), and 2-DR-FW-102 bp-MOD: 5′-CTAATGAGTGAGCTAATCTCmUGTCAGCGGCACTGCCTATACAGCG-3′ (SEQ ID NO:______).

These three fragments were cloned into pUC19-2CD that was opened using PUC19-anti-3-MOD: 5′-AGAGATTAGCTCACTCATTAmGGCACCCCAGGCTTTACACTTTATGCTTC-3′ (SEQ ID NO:______), and 2-pUC19-4-DR-sense-3-MOD: 5′-CACTTCTTTGACATTGGAGGmACCAGCTGCATTAATGAATCGGCCAAC-3′ (SEQ ID NO:______).

The annealed fragments were transformed into Sure cells and selected on CG-Amp. The resulting plasmids were named pUC19-2CD-45 bp, pUC19-2CD-78 bp, pUC19-2CD-102 bp.

The downstream flanking sequence was amplified from genomic DNA of OKB105 cells using primers: 2-G: 5′-GTCACGCTGAACCTGAACATTTCCGATCAAATC-3′ (SEQ ID NO:______), and 2-H: 5′-CACTTCTTTGACATTGGCGGACATTCATTAGC-3′ (SEQ ID NO:______).

This DNA fragment was subsequently amplified with primers: 2-DR-HG-FW: 5′-CATTCTTTAGCTGGTATGAAmGATGCCTGCCTTGGTTCATCAAGAACTGG-3′ (SEQ ID NO:______), and 2-HG-4-vect+DR-BK: 5′-GAGTGCAGAATACTCAAACCmGGTCACGCTGAACCTGAACATTTCCGATC-3′ (SEQ ID NO:______).

This fragment was annealed separately to each of the PCR products derived from amplifying pUC19-2CD-45 bp, pUC19-2CD-78 bp, pUC19-2CD-102 bp with 2-DR-in-Vect-4-HG-BK#1: 5′-CTTCATACCAGCTAAAGAATmGTCCTCCAATGTCAAAGAAGTGGTCAAAG-3′ (SEQ ID NO:______), and Vector-FW-4-HG-1&2: 5′-CGGTTTGAGTATTCTGCACTmCTTCCGCTTCCTCGCTCACTGACTC-3′ (SEQ ID NO:______).

The annealed fragments were transformed into Sure cells and selected on CG-Amp. The resulting plasmids were named pUC19-2CD-45DR-2HG, pUC19-2CD-78DR-2HG, pUC19-2CD-102DR-2HG.

We observed that when trying to identify plasmids pUC19-2CD-45 bp, pUC19-2CD-78 bp, pUC19-2CD-102 bp, pUC19-2CD-45DR-2HG, pUC19-2CD-78DR-2HG, pUC19-2CF-102DR-2HG, some candidate colonies underwent recombination between the 45 bp, 78 bbp, and 102 bp direct repeats. Nonetheless, we were able to identify the desired sequences.

However, when we tried to insert the upp-kan cassette in between the 2CD sequence and the DR, we were unable to construct such plasmid. Therefore, we decided to join the upp-kan cassette using restriction enzymes and T4 DNA ligase to engineer three constructs.

The upstream flanking sequence was obtained using the primers 1CD-and-2CD-FW: 5′-GGATGTGCTGCAAGGCGATTAAGTTGGGTCTG-3′ (SEQ ID NO:______), and 2CD-BstXI-BK: 5′-ATGCTAATCCACTCTCTTGGGTCAAAGATACCAGCCTTCTCAACG-3′ (SEQ ID NO:______).

The upp-kan cassette was obtained by amplifying from pUC-UPP-KAN using the primers UPP-KAN-BstXI-FW: 5′-ATGCTAAGCCAAGAGAGTGGGTTTTTTGACGATGTTCTTGAAACTCAATG-3′ (SEQ ID NO:______), and UPP-KAN-and-KAN-BglI-BK: 5′-ATATCTGAGCCAGAGAGGCACCAATCAAAAAACAGATGGCCGCTATTAAAGC-3′ (SEQ ID NO:______).

The downstream sequence was obtained from pUC19-2CD-45DR-2HG, pUC19-2CD-78DR-2HG, pUC19-2CD-102DR-2HG using primers 1HG-and-2HG-BglI-FW: 5′-ATTACTACGCCTCTCTGGCACACAACATACGAGCCGGAAGCATAAAGTG-3′ (SEQ ID NO:______), and 1HG-and-2HG-BK: 5′-TGATTCTGTGGATAACCGTATTACCGCCTTTGAGTG-3′ (SEQ ID NO:______).

The upstream flanking sequence and the UPP-KAN fragment were digested with BstXI and ligated with T4DNA ligase. The ligation reaction was then used as a template to re-amplify the ligated product using 1CD-and-2CD-FW and UPP-KAN-and-KAN-BglI-BK.

The resulting PCR product as well as the downstream flanking sequence were digested with BglI and ligated. The ligation mixtures 2CD-UPP-KAN-45DR-2HG, 2CD-UPP-KAN-78DR-2HG, and 2CD-UPP-KAN-102DR-2HG were cleaned using Qiagen's per purification kit and transformed into competent OKB105 Δupp Spect^{R} cells and plated on LB containing 30 μg/ml kanamycin plates. We obtained colonies from the first ligation mixture. These colonies were inoculated in LB with 25 μg/ml thymine and grown o/n. Cells were then washed in 0.5% glucose and plated on M9YE.

Table 3 lists the fusion points for polypeptides produced by this method.

TABLE 3
Fusion points and substituted sequences in
thiolation domain substitutions at modules 2

| | | | | Surfactin |
|---|---|---|---|---|
| | | | | sequence |
| | Surfactin sequence | N-terminus | C-terminus | downstream of |
| | upstream of | substituted | substituted | substituted |
| Strain name | substituted module | sequence | sequence | module |
| 16923_G4 | ..LNVEKAGIFD.. | ..HFFELGGHSL.. | ..LGVSGIGILD.. | ..HFFDIGGHSL.. |
| 16612_H2 | ..LNVEKAGIFD.. | ..NFFELGGHSL.. | ..LGVETIGVHD.. | ..HFFDIGGHSL.. |
| 18499_B7 | ..LNVEKAGIFD.. | ..HFFTLGGHSL.. | ..LGISGVGVLD.. | ..HFFDIGGHSL.. |

One of the clones has a point mutation that replaces P with L (shown in bold and italics in Table 4, below), which increases the yield of the surfactin analog with respect to the construct that does not have that mutation.

TABLE 4
Upstream and downstream boundaries of fusion points
in thiolation domain deletion of module 2

| Strain | Surfactin sequence upstream of | Surfactin sequence downstream of |
|---|---|---|
| name | fusion point of deleted module 2 | fusion point of deleted module 2 |
| 14311_D3 | KAIAAIWQDVLNVEKAGIFD | HFFDIGGHSLAGMKM [symbol] ALVH |
| 14311_F6 | KAIAAIWQDVLNVEKAGIFD | HFFDIGGHSLAGMKMPALVH |

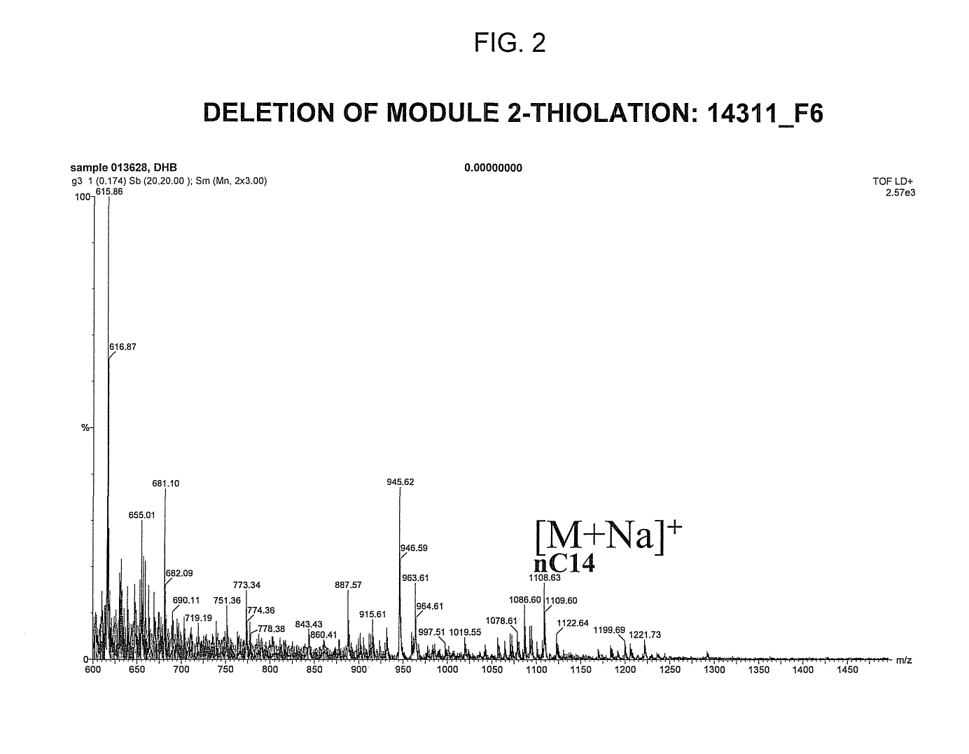

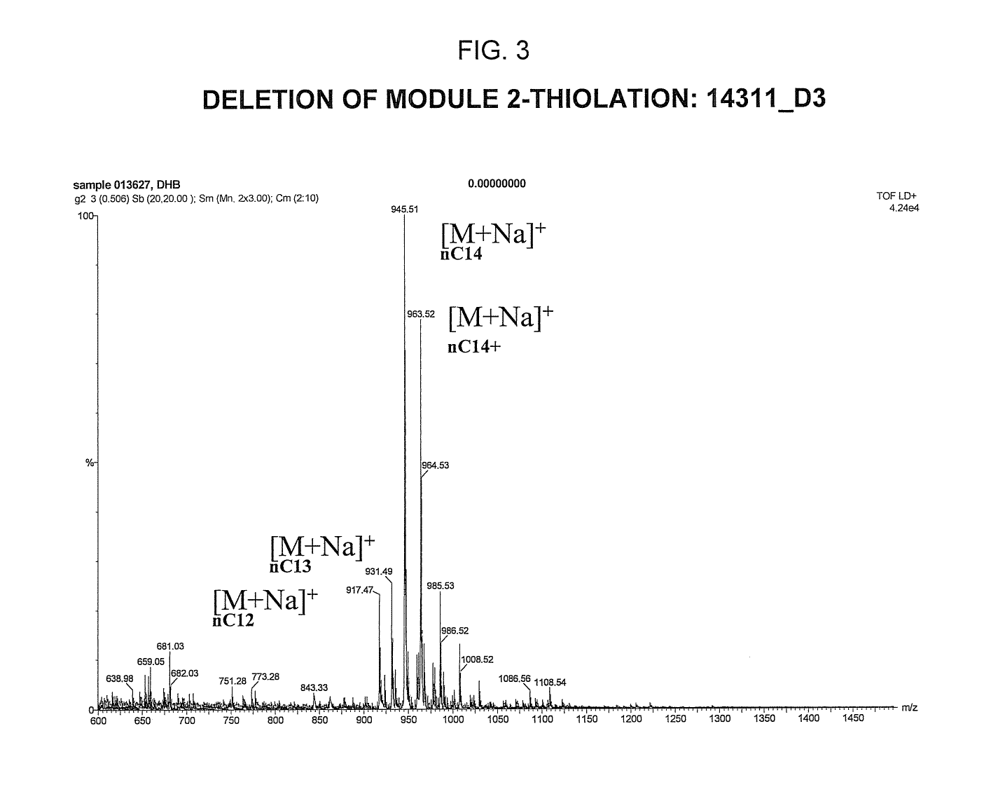

In this experiment, we observed that 4 out 48 constructs had a recombination event between the direct repeats. Of those 4, one had the desired sequence, strain 013627 (FIG. 2), and one had point mutations, strain 013628 (FIG. 3). However, both constructs were able to produce a 6-member (surfactin-analog) ring, as judged by mass spectrometry analysis. The strain harboring the gene with the point mutation produced more of the compound.

### Example 3

### Engineering Lipopeptide Synthetase Polypeptides with a Deletion of an Adenylation Domain and a Thiolation Domain

We engineered a recombinant lipopeptide synthetase polypeptide in which the highly variable sequences in the condensation domains of modules 1 and 2 of the first surfactin synthetase were joined. The goal in generating this recombinant polypeptide was to produce a 6-member (surfactin-analog) ring lacking the amino acid L-glutamic acid.

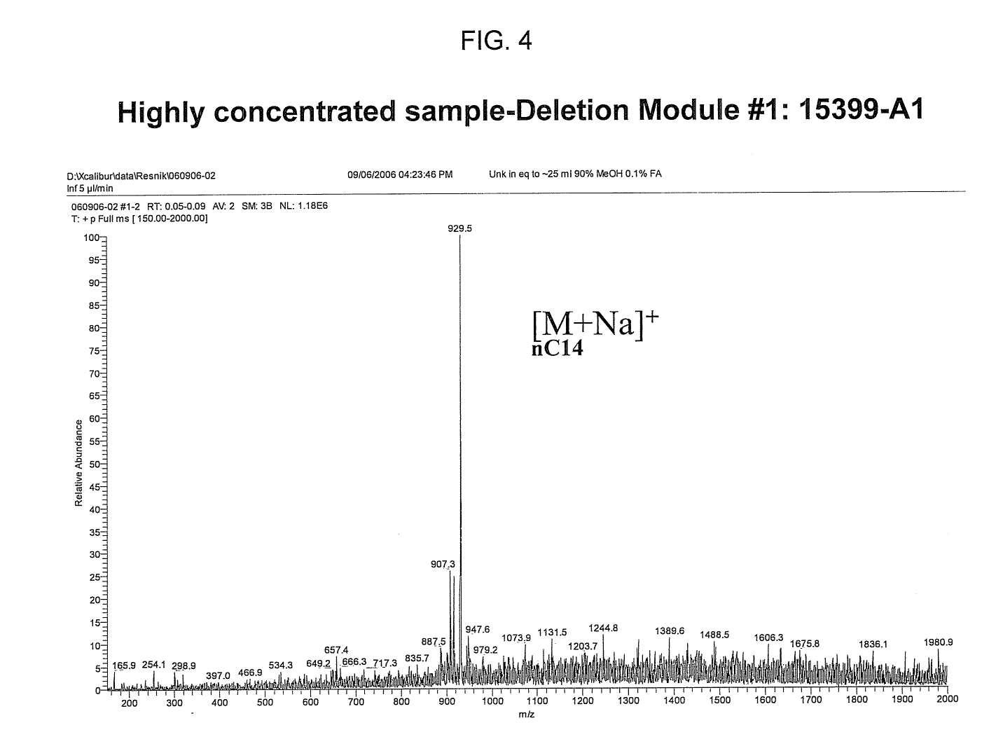

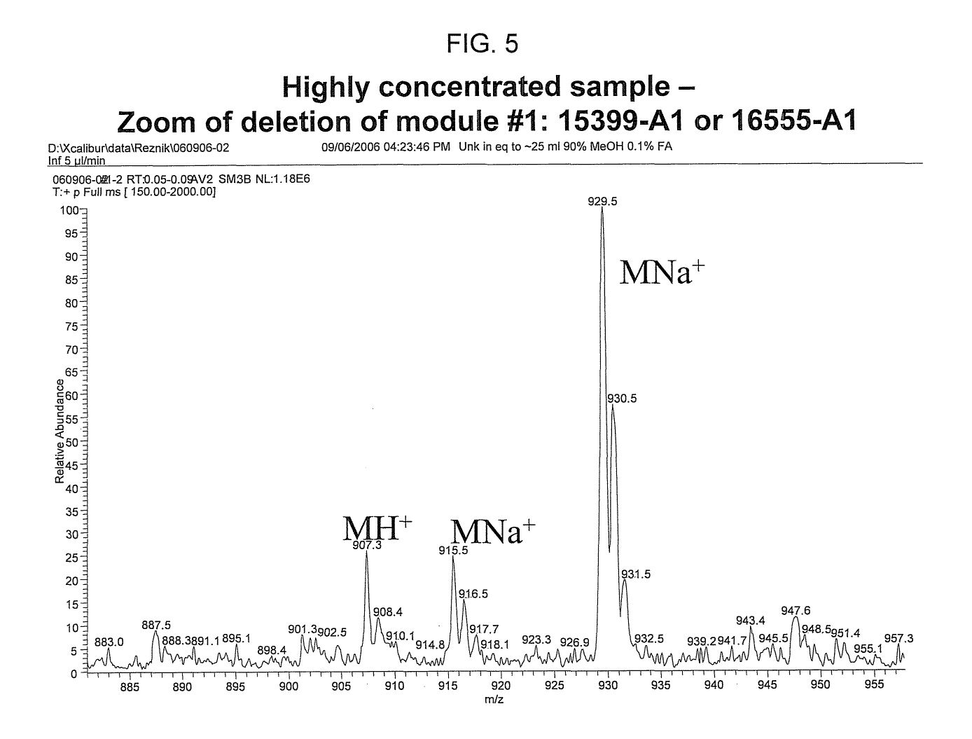

We discovered that polypeptides engineered in this manner produced a small molecule that had a six amino acid ring with the beta hydroxy fatty acid attached to Leu. Natural surfactin is a seven amino acid ring in which the fatty acid is attached to Glu. Thus, we successfully produced an engineered polypeptide that produced a cyclic surfactin analog (FIG. 4 and FIG. 5).

The recombinant polypeptides that were produced, as described further below, were hybrid modules. The polypeptides contained a fusion of amino acids of the first module of the SRFA protein with amino acids of module two of the SRFA protein. Table 5 and Table 6 list sequences at the fusion points for the polypeptides that were made.

TABLE 5
Upstream and downstream boundaries of fusion points in C domain deletions of
module 1 of surfactin synthetase

| Strains | | |
|---|---|---|
| that | | |
| delete | | |
| module 1 | | |
| (L-Glu). | | |
| Strains | | |
| on plate | Surfactin synthetase sequence upstream of | Surfactin synthetase sequence downstream of |
| 15399 | fusion point of deleted module 1 | fusion point of deleted module 1 |
| A1 | PEADAELIDLDQAIEEGAEESLNAD | ADEEESYHADARNLALPLDSAAMANLTY |
| D1 | PEADAELIDLDQAIEEGAEESLNAD | EEESYHADARNLALPLDSAAMANLTY |
| E1 | PEADAELIDLDQAIEEGAEESLNA | ADARNLALPLDSAAMANLTY |
| F1 | PEADAELIDLDQAIEEGAEESLNA | RNLALPLDSAAMANLTY |
| G1 | PEADAELIDLDQAIEEGAEESLN | DEEESYHADARNLALPLDSAAMANLTY |
| H1 | PEADAELIDLDQAIEEGAEESLN | EESYHADARNLALPLDSAAMANLTY |
| A2 | PEADAELIDLDQAIEEGAEESLN | SYHADARNLALPLDSAAMANLTY |
| B2 | PEADAELIDLDQAIEEGAEESLN | HADARNLALPLDSAAMANLTY |
| C2 | PEADAELIDLDQAIEEGAEESLN | ADARNLALPLDSAAMANLTY |
| D2 | PEADAELIDLDQAIEEGAEESLN | RNLALPLDSAAMANLTY |
| E2 | PEADAELIDLDQAIEEGAEESLN | ALPLDSAAMANLTY |
| F2 | PEADAELIDLDQAIEEGAEES | DEEESYHADARNLALPLDSAAMANLTY |
| G2 | PEADAELIDLDQAIEEGAEES | EESYHADARNLALPLDSAAMANLTY |
| H2 | PEADAELIDLDQAIEEGAEES | SYHADARNLALPLDSAAMANLTY |
| C3 | PEADAELIDLDQAIEEGAEES | YHADARNLALPLDSAAMANLTY |
| D3 | PEADAELIDLDQAIEEGAEES | RNLALPLDSAAMANLTY |
| E3 | PEADAELIDLDQAIEEGAEES | LALPLDSAAMANLTY |
| F3 | PEADAELIDLDQAIEEGAEE | EESYHADARNLALPLDSAAMANLTY |
| G3 | PEADAELIDLDQAIEEGAEE | SYHADARNLALPLDSAAMANLTY |
| H3 | PEADAELIDLDQAIEEGAEE | HADARNLALPLDSAAMANLTY |
| B4 | PEADAELIDLDQAIEEGAEE | RNLALPLDSAAMANLTY |
| C4 | PEADAELIDLDQAIEEGAEE | LALPLDSAAMANLTY |
| D4 | PEADAELIDLDQAIEEG | DEEESYHADARNLALPLDSAAMANLTY |
| E4 | PEADAELIDLDQAIEEG | EESYHADARNLALPLDSAAMANLTY |
| F4 | PEADAELIDLDQAIEEG | SYHADARNLALPLDSAAMANLTY |
| G4 | PEADAELIDLDQAIEEG | HADARNLALPLDSAAMANLTY |
| H4 | PEADAELIDLDQAIEEG | ADARNLALPLDSAAMANLTY |
| A5 | PEADAELIDLDQAIEEG | RNLALPLDSAAMANLTY |

TABLE 6
Upstream and downstream boundaries of fusion points in C domain deletions of
module 2 of surfactin synthetase

| Strains that | | |
|---|---|---|
| delete | | |
| module 2 (L- | | |
| Leu). Strains | | |
| on plate | Surfactin sequence upstream of fusion point | Surfactin sequence downstream of |
| 15399 | of deleted module 2 | fusion point of deleted module 2 |
| A6 | ADEEESYHADARNLALPLDSAAMANL | EENPENPE |
| E5 | ADEEESYHADARNLALPLDSAAMAN | RTILSLPLDENDEENPENPE |
| G5 | ADEEESYHADARNLALPLDSAAMAN | PLDENDEENPENPE |
| F5 | ADEEESYHADARNLALPLDSAAMAN | SLPLDENDEENPENPE |
| C6 | ADEEESYHADARNLALPLDSAAMAN | ENPE |
| C7 | ADEEESYHADARN | ENPE |
| F7 | ADEEESYHADA | RTILSLPLDENDEENPENPE |
| B6 | ADEEESYHADARNLALPLDSAAMANL | NPENPE |
| H5 | ADEEESYHADARNLALPLDSAAMANL | DENDEENPENPE |
| D8 | ADEEESYHADA | ENPE |
| G6 | ADEEESYHADARN | PLDENDEENPENPE |

Because of the large number of potential joining locations, we decided to establish a protocol that could easily be automated to generate multiple candidates. To that effect, we replaced the approximate region of chromosomal DNA to be deleted with a construct containing an “upp-kanamycin” cassette. In this construct, the cassette was flanked by sequence homologous to the DNA upstream of the variable region of condensation domain of module 1 cassette and sequence homologous to the DNA downstream of the variable region of condensation domain of module 2. Deletions were established by joining the 3′-end of an approximately 1.3 kb region of the variable region of condensation domain of module 1 cassette and the 5′-end of an approximately 1.3 kb region of the variable region of condensation domain of module 2 in pUC19. Then, by site-directed deletions at the junction of the variable condensations domain regions, 28 plasmids were engineered to establish various boundaries between these regions. These plasmids were separately transformed into Bacillus subtilis competent cells.

Several colonies were picked following 18 hr incubation at 37° C. or 36 hr at 30° C. and grown in liquid media (LB with 25 μg/ml thymine and 100 μg/ml spectinomycin). Then, small aliquots of these cells were replica-plated on LB with 25 μg/ml thymine and 100 μg/ml spectinomycin, and LB with 30 μg/ml kanamycin. Cells that grew in the first plate but not in the one containing kanamycin were sequenced, since in those, it was likely that a recombination event replaced the “upp-kanamycin” cassette with the plasmid carrying the engineered boundaries between variable condensation domains of modules 1 and 2. The efficiency of selecting colonies by replica plating varied between 10-60%. Successful Bacillus subtilis constructs were grown in LB with 25 μg/ml thymine and the small molecules that were produced and secreted to the media were analyzed by MALDI.

Due to the high similarity that exists among modules, it was advantageous to perform nested PCR reactions to amplify genomic DNA sequences. The template for the upstream flanking sequence of the upp-kan cassette was amplified from genomic DNA of OKB105 cells using primers VP-3C-sense-1: 5′-TATTGTCGGGAATGCGATCATG-3′ (SEQ ID NO:______), and VP-3D-anti-1:5′-AGATTCAACCAAATGATGAACCTG-3′ (SEQ ID NO:______).

This PCR product was named 3CD and was used to generate the fragment that was used to ligate to the upp-kan cassette using primers VP-3C-sense-1: 5′-TATTGTCGGGAATGCGATCATG-3′ (SEQ ID NO:______), and 3CD-BSTXI-BK: 5′-ATGTGCTACCACTCCTCTGGATCAGCATTCAGGCTTTCTTCTGCACC-3′ (SEQ ID NO:______).

The upp-kan fragment was obtained from pUC19-UPP-KAN using primers 3-4-UPP-KAN-BSTXI-FW: 5′-ATGCTAAGCCAGAGGAGTGGGTTTTTTGACGATGTTCTTGAAACTCAATG-3′ (SEQ ID NO:______), and 3-4-UPP-KAN-BSTXI-BK: 5′-ATGTGCTACCAACTTCCTGGCAGAGTATGGACAGTTGCGGATGTACTTCAG-3′(SEQ ID NO:______).

The downstream template for the flanking sequence of the upp-kan cassette was amplified from genomic DNA of OKB105 cells using primers VP-3H-sense-1: 5′-ATGCAGCATTTCTTCCGTGACAGC-3′ (SEQ ID NO:______), and VP-3G-anti-1: 5′-GCAGCTCGTCCATTTGGATAAACACC-3′ (SEQ ID NO:______).

This PCR product was named 3HG and was used to generate the fragment that was used to ligate to the upp-kan cassette using primers 3HG-BSTXI-FW: 5′-ATATCTGTCCAGGAAGTTGGGCCGATGAGGAAGAAAGCTATCATGC-3′ (SEQ ID NO:______), and VP-3G-anti-1: 5′-GCAGCTCGTCCATTTGGATAAACACC-3′ (SEQ ID NO:______).

All three fragments were separately digested with BstXI and ligated in a one step reaction. The ligation mixture was cleaned using Qiagen's PCR purification kit and transformed into OKB 105 Δupp Spect^{R} cells and plated on LB containing 30 μg/ml kanamycin plates.

Colonies were screened by PCR-mapping and sequencing. The “upp-kan” marked strain was named OKB105 Δupp Spect^{R} upp⁺ kan^{R} (Proj3).

Plasmid Used to Generate Deletions

Deletions were established by joining the 3′-end of an approximately 1.3 kb region of the variable region of condensation domain of module 1 cassette and the 5′-end of an approximately 1.3 kb region of the variable region of condensation domain of module 2 in pUC19.

The template for generating the upstream flanking sequence was obtained by using 3CD as a template with primers 3CD-FW: 5′-AAACAATTTGAATCTGTGCCmUGAACTTGTCTCTTTGAAACGGAATGCATC-3′ (SEQ ID NO:______), and 3CD-BK: 5′-ATAGCTTTCTTCCTCATCGGmCATCAGCATTCAGGCTTTCTTCTGCACC-3′ (SEQ ID NO:______), and cloning this fragment into pUC19 that was opened using primers pUC19 sense-6: 5′-GCCGATGAGGAAGAAAGCTAmUACCAGTGAATTAGAGCTCGGTACC-3′ (SEQ ID NO:______), and pUC19 anti-6: 5′-AGGCACAGATTCAAATTGTTmUACCCAACTTAATCGCCTTGCAGCACATC-3′ (SEQ ID NO:______).

Both fragments were annealed and transformed into Sure cells and plated on CG-Amp. The resulting plasmid was named pUC19-3CD.

The template for generating the downstream flanking sequence was obtained by using 3HG as a template with primers 3HG-FW: 5′-GCCGATGAGGAAGAAAGCTAmUCATGCAGACGCAAGAAATCTCGCACTGCC-3′ (SEQ ID NO:______), and 3HG-BK: 5′-AATTTTTCCATTCCCTGTCAmGCGGCAGCTCGTCCATTTGGATAAACAC-3′ (SEQ ID NO:______).

This PCR product was cloned into plasmid pUC19-3CD that was opened using primers pUC19-sense-7: 5′-CTGACAGGGAATGGAAAAATmUTTACGCTTACTCGCTCACTGACTC-3′ (SEQ ID NO:______), and pUC19-anti-7: 5′-ATAGCTTTCTTCCTCATCGGmCATCAGCATTCAGGCTTTCTTCTGCACC-3′ (SEQ ID NO:______).

Both fragments were annealed and transformed into Sure cells and plated on CG-Amp. The resulting plasmid was named pUC19-3CD-3HG.

This plasmid was then used as a template to generate 28 fusion points between the 3′-end of 3CD and the 5′-end of 3HG. Each fusion point was engineered using pairs of primers listed below.

2082-1:A1:
(SEQ ID NO: )
5′-GAATGCTGATGAGGAAGAAAmGCTATCATGCAGACGCAAGAAATCTCG-3′
2082-2:A1:
(SEQ ID NO: )
5′-CTTTCTTCCTCATCAGCATTmCAGGCTTTCTTCTGCACCTTCCTCA-3′
2082-1:D1:
(SEQ ID NO: )
5′-AAGAAAGCCTGAATGCTGCAmGACGCAAGAAATCTCGCACTGCCTC-3′
2082-2:D1:
(SEQ ID NO: )
5′-CTGCAGCATTCAGGCTTTCTmUCTGCACCTTCCTCAATCGCCTGAT-3′
2082-1:E1:
(SEQ ID NO: )
5′-AAGCCTGAATGCTAGAAATCmUCGCACTGCCTCTTGATTCTGCAGC-3′
2082-2:E1:
(SEQ ID NO: )
5′-AGATTTCTAGCATTCAGGCTmUTCTTCTGCACCTTCCTCAATCGCC-3′
2082-1:F1:
(SEQ ID NO: )
5′-AAGAAAGCCTGAATGCTCTCmGCACTGCCTCTTGATTCTGCAGCAA-3′
2082-2:F1:
(SEQ ID NO: )
5′-CGAGAGCATTCAGGCTTTCTmUCTGCACCTTCCTCAATCGCCTGAT-3′
2082-1:G1:
(SEQ ID NO: )
5′-GAATGATGAGGAAGAAAGCTAmUCATGCAGACGCAAGAAATCTCGCA-3′
2082-2:G1:
(SEQ ID NO: )
5′-ATAGCTTTCTTCCTCATCATTmCAGGCTTTCTTCTGCACCTTCCTCA-3′
2082-1:H1:
(SEQ ID NO: )
5′-GAATGAAGAAAGCTATCATGmCAGACGCAAGAAATCTCGCACTGCC-3′
2082-2:H1:
(SEQ ID NO: )
5′-GCATGATAGCTTTCTTCATTmCAGGCTTTCTTCTGCACCTTCCTCA-3′
2082-1:A2:
(SEQ ID NO: )
5′-AAAGCCTGAATAGCTATCATmGCAGACGCAAGAAATCTCGCACTGC-3′
2082-2:A2:
(SEQ ID NO: )
5′-CATGATAGCTATTCAGGCTTmUCTTCTGCACCTTCCTCAATCGCCT-3′
2082-1:B2:
(SEQ ID NO: )
5′-CAGAAGAAAGCCTGAATCATmGCAGACGCAAGAAATCTCGCACTGC-3′
2082-2:B2:
(SEQ ID NO: )
5′-CATGATTCAGGCTTTCTTCTmGCACCTTCCTCAATCGCCTGATCTAA-3′
2082-1:C2:
(SEQ ID NO: )
5′-GAAGAAAGCCTGAATGCAGAmCGCAAGAAATCTCGCACTGCCTCTT-3′
2082-2:C2:
(SEQ ID NO: )
5′-GTCTGCATTCAGGCTTTCTTmCTGCACCTTCCTCAATCGCCTGATC-3′
2082-1:D2:
(SEQ ID NO: )
5′-AGAAGAAAGCCTGAATAGAAAmUCTCGCACTGCCTCTTGATTCTGCA-3′
2082-2:D2:
(SEQ ID NO: )
5′-ATTTCTATTCAGGCTTTCTTCmUGCACCTTCCTCAATCGCCTGATCTA-3′
2082-1:E2:
(SEQ ID NO: )
5′-CAGAAGAAAGCCTGAATCTCmGCACTGCCTCTTGATTCTGCAGCAA-3′
2082-2:E2:
(SEQ ID NO: )
5′-CGAGATTCAGGCTTTCTTCTmGCACCTTCCTCAATCGCCTGATCTAA-3′
2082-1:F2:
(SEQ ID NO: )
5′-AGAAAGCGATGAGGAAGAAAmGCTATCATGCAGACGCAAGAAATCTCG-3′
2082-2:F2:
(SEQ ID NO: )
5′-CTTTCTTCCTCATCGCTTTCmUTCTGCACCTTCCTCAATCGCCTGA-3′
2082-1:G2:
(SEQ ID NO: )
5-GAAGAAAGCGAAGAAAGCTAmUCATGCAGACGCAAGAAATCTCGCA-3′
2082-2:G2:
(SEQ ID NO: )
5′-ATAGCTTTCTTCGCTTTCTTmCTGCACCTTCCTCAATCGCCTGATC-3′
2082-1:H2:
(SEQ ID NO: )
5′-CAGAAGAAAGCAGCTATCATmGCAGACGCAAGAAATCTCGCACTGC-3′
2082-2:H2:
(SEQ ID NO: )
5′-CATGATAGCTGCTTTCTTCTmGCACCTTCCTCAATCGCCTGATCTAA-3′
2082-1:C3:
(SEQ ID NO: )
5′-GCAGAAGAAAGCAGAAATCTmCGCACTGCCTCTTGATTCTGCAGCA-3′
2082-2:C3:
(SEQ ID NO: )
5′-GAGATTTCTGCTTTCTTCTGmCACCTTCCTCAATCGCCTGATCTAAGT-3′
2082-1:D3:
(SEQ ID NO: )
5′-AAGGTGCAGAAGAAAGCCTCmGCACTGCCTCTTGATTCTGCAGCAA-3′
2082-2:D3:
(SEQ ID NO: )
5′-CGAGGCTTTCTTCTGCACCTmUCCTCAATCGCCTGATCTAAGTCAATCA-3′
2082-1:E3:
(SEQ ID NO: )
5′-AAGAAGATGAGGAAGAAAGCmUATCATGCAGACGCAAGAAATCTCG-3′
2082-2:E3:
(SEQ ID NO: )
5′-AGCTTTCTTCCTCATCTTCTmUCTGCACCTTCCTCAATCGCCTGAT-3′
2082-1:F3:
(SEQ ID NO: )
5′-CAGAAGAAGAAGAAAGCTATCAmUGCAGACGCAAGAAATCTCGCACTG-3′
2082-2:F3:
(SEQ ID NO: )
5′-ATGATAGCTTTCTTCTTCTTCTmGCACCTTCCTCAATCGCCTGATCTAA-3′
2082-1:G3:
(SEQ ID NO: )
5′-GAAGAAAGCTATCATGCAGAmCGCAAGAAATCTCGCACTGCCTCTT-3′
2082-2:G3:
(SEQ ID NO: )
5′-GTCTGCATGATAGCTTTCTTmCTGCACCTTCCTCAATCGCCTGATC-3′
2082-1:H3:
(SEQ ID NO: )
5′-AGGAAGGTGCAGAAGAACATmGCAGACGCAAGAAATCTCGCACTGC-3′
2082-2:H3:
(SEQ ID NO: )
5′-CATGTTCTTCTGCACCTTCCmUCAATCGCCTGATCTAAGTCAATCAGC-3′
2082-1:B4:
(SEQ ID NO: )
5′-AGGTGCAGAAGAAAGAAATCmUCGCACTGCCTCTTGATTCTGCAGC-3′
2082-2:B4:
(SEQ ID NO: )
5′-AGATTTCTTTCTTCTGCACCmUTCCTCAATCGCCTGATCTAAGTCAATC-3′
2082-1:C4:
(SEQ ID NO: )
5′-AGAAGAACTCGCACTGCCTCmUTGATTCTGCAGCAATGGCCAACCT-3′
2082-2:C4:
(SEQ ID NO: )
5′-AGAGGCAGTGCGAGTTCTTCmUGCACCTTCCTCAATCGCCTGATCTA-3′
2082-1:D4:
(SEQ ID NO: )
5′-AGGTGATGAGGAAGAAAGCTmATCATGCAGACGCAAGAAATCTCGC-3′
2082-2:D4:
(SEQ ID NO: )
5′-TAGCTTTCTTCCTCATCACCmUTCCTCAATCGCCTGATCTAAGTCAATC-3′
2082-1:E4:
(SEQ ID NO: )
5′-GAGGAAGGTGAAGAAAGCTAmUCATGCAGACGCAAGAAATCTCGCA-3′
2082-2:E4:
(SEQ ID NO: )
5′-ATAGCTTTCTTCACCTTCCTmCAATCGCCTGATCTAAGTCAATCAGCTC-3′
2082-1:F4:
(SEQ ID NO: )
5′-GAAGGTAGCTATCATGCAGAmCGCAAGAAATCTCGCACTGCCTCTT-3′
2082-2:F4:
(SEQ ID NO: )
5′-GTCTGCATGATAGCTACCTTmCCTCAATCGCCTGATCTAAGTCAATCAG-3′
2082-1:G4:
(SEQ ID NO: )
5′-ATTGAGGAAGGTCATGCAGAmCGCAAGAAATCTCGCACTGCCTCTT-3′
2082-2:G4:
(SEQ ID NO: )
5′-GTCTGCATGACCTTCCTCAAmUCGCCTGATCTAAGTCAATCAGCTCA-3′
2082-1:H4:
(SEQ ID NO: )
5′-AGGTGCAGACGCAAGAAATCmUCGCACTGCCTCTTGATTCTGCAGC-3′
2082-2:H4:
(SEQ ID NO: )
5′-AGATTTCTTGCGTCTGCACCmUTCCTCAATCGCCTGATCTAAGTCAA-3′
2082-1:A5:
(SEQ ID NO: )
5′-GATTGAGGAAGGTAGAAATCTmCGCACTGCCTCTTGATTCTGCAGCA-3′
2082-2:A5:
(SEQ ID NO: )
5′-GAGATTTCTACCTTCCTCAATmCGCCTGATCTAAGTCAATCAGCTCAGC-3′.

The annealed fragments were transformed into Sure cells and selected on CG-Amp. The resulting plasmids were named pUC19-Del-Mod1-A1, pUC19-Del-Mod1-D1, pUC19-Del-Mod1-E1, pUC19-Del-Mod1-F1, pUC19-Del-Mod1-G1, pUC19-Del-Mod1-H1, pUC19-Del-Mod1-A2, pUC19-Del-Mod1-B2, pUC19-Del-Mod1-C2, pUC19-Del-Mod1-D2, pUC19-Del-Mod1-E2, pUC19-Del-Mod1-F2, pUC19-Del-Mod1-G2, pUC19-Del-Mod1-H2, pUC19-Del-Mod1-C3, pUC19-Del-Mod1-D3, pUC19-Del-Mod1-E3, pUC19-Del-Mod1-F3, pUC19-Del-Mod1-G3, pUC19-Del-Mod1-H3, pUC19-Del-Mod1-B4, pUC19-Del-Mod1-C4, pUC19-Del-Mod1-D4, pUC19-Del-Mod1-E4, pUC19-Del-Mod1-F4, pUC19-Del-Mod1-G4, pUC19-Del-Mod1-H4, pUC19-Del-Mod1-A5.

These plasmids were transformed into OKB105 Δupp Spect^{R} upp⁺ kan^{R} (Proj3) to yield strains 15399-A1, 15399-D1, 15399-E1, 15399-F1, 15399-G1, 15399-H1, 15399-A2, 15399-B2, 15399-C2, 15399-D2, 15399-E2, 15399-F2, 15399-G2, 15399-H2, 15399-C3, 15399-D3, 15399-E3, 15399-F3, 15399-G3, 15399-H3, 15399-B4, 15399-C4, 15399-D4, 15399-E4, 15399-F4, 15399-G4, 15399-H4, 15399-A5. Yield of compound from these strains was low. FIG. 4 and FIG. 5 show MALDI analysis of compounds produced by two of the strains. Table 7 lists the strains, products, yield, and types of substitutions described in this Example, and in other Examples herein. Table 8 lists the amino acid composition of surfactin and surfactin analogs produced by engineered polypeptides and strains described in this Example and other Examples herein.

TABLE 7
Engineered lipopeptide synthetase-producing strains

| | | | | Substitution |
|---|---|---|---|---|
| Strain name | Product | Lab name | Yield | mode/AA(#)/Source |
| OKB105 | Wildtype surfactin | OKB105 | Good | |
| 14311_D3 | Deletion of module 2 (L- | Δmod2-P->L | Good | Thiolation |
| | Leu) | | | |
| 14311_F6 | Deletion of module 2 (L- | Δmod2 | Low | Thiolation |
| | Leu) | | | |
| 16923_G4 | Substitution of L-Leu w/ | Tyr | Good | Thiolation/Tyr(7) |
| | L-Tyr @ position 2 | | | Tyrocidine |
| 16612_H2 | Substitution of L-Leu w/ | | Lower than | Thiolation/D-Leu(4) |
| | L-Leu @ position 2 | | WT | Linear gramicidin |
| | | | OKB105 | |
| 18499_B7 | Substitution of L-Leu w/ | Phe | Low | Thiolation/L-Phe(3) |
| | L-Phe @ position 2 | | | Tyrocidine |
| 15399-A1, | Deletions of module 1 (L- | | Low | Condensation |
| 15399-D1, | Glu) | | | |
| 15399-E1, | | | | |
| 15399-F1, | | | | |
| 15399-G1, | | | | |
| 15399-H1, | | | | |
| 15399-A2, | | | | |
| 15399-B2, | | | | |
| 15399-C2, | | | | |
| 15399-D2, | | | | |
| 15399-E2, | | | | |
| 15399-F2, | | | | |
| 15399-G2, | | | | |
| 15399-H2, | | | | |
| 15399-C3, | | | | |
| 15399-D3, | | | | |
| 15399-E3, | | | | |
| 15399-F3, | | | | |
| 15399-G3, | | | | |
| 15399-H3, | | | | |
| 15399-B4, | | | | |
| 15399-C4, | | | | |
| 15399-D4, | | | | |
| 15399-E4, | | | | |
| 15399-F4, | | | | |
| 15399-G4, | | | | |
| 15399-H4, | | | | |
| 15399-A5 | | | | |
| 15399-B6, | Deletions of module 2 (L- | | Very Low | Condensation |
| 15399-E5, | Leu) | | | |
| 15399-G5, | | | | |
| 15399-F5, | | | | |
| 15399-C6, | | | | |
| 15399-C7, | | | | |
| 15399-F7, | | | | |
| 15399-A6, | | | | |
| 15399-H5, | | | | |
| 15399-D8, | | | | |
| 15399-G6 | | | | |
| 23960_A1 | Deletion of modules 2-7 | | Low | Thiolation |

TABLE 8
Amino acid composition of surfactin and surfactin analogs

| | | | | | | | | Total |
|---|---|---|---|---|---|---|---|---|
| Strain name | AA1 | AA2 | AA3 | AA4 | AA5 | AA6 | AA7 | AAs |
| OKB105 | L-Glu | L-Leu | D-Leu | L-Val | L-Asp | D-Leu | L-Leu | 7 |
| 14311_D3 | L-Glu | | D-Leu | L-Val | L-Asp | D-Leu | L-Leu | 6 |
| 14311_F6 | L-Glu | | D-Leu | L-Val | L-Asp | D-Leu | L-Leu | 6 |
| 16923_G4 | L-Glu | L-Tyr | D-Leu | L-Val | L-Asp | D-Leu | L-Leu | 7 |
| 16612_H2 | L-Glu | L-Leu | D-Leu | L-Val | L-Asp | D-Leu | L-Leu | 7 |
| 18499_B7 | L-Glu | L-Phe | D-Leu | L-Val | L-Asp | D-Leu | L-Leu | 7 |
| 15399-A1, | | L-Leu | D-Leu | L-Val | L-Asp | D-Leu | L-Leu | 6 |
| 15399-D1, | | | | | | | | |
| 15399-E1, | | | | | | | | |
| 15399-F1, | | | | | | | | |
| 15399-G1, | | | | | | | | |
| 15399-H1, | | | | | | | | |
| 15399-A2, | | | | | | | | |
| 15399-B2, | | | | | | | | |
| 15399-C2, | | | | | | | | |
| 15399-D2, | | | | | | | | |
| 15399-E2, | | | | | | | | |
| 15399-F2, | | | | | | | | |
| 15399-G2, | | | | | | | | |
| 15399-H2, | | | | | | | | |
| 15399-C3, | | | | | | | | |
| 15399-D3, | | | | | | | | |
| 15399-E3, | | | | | | | | |
| 15399-F3, | | | | | | | | |
| 15399-G3, | | | | | | | | |
| 15399-H3, | | | | | | | | |
| 15399-B4, | | | | | | | | |
| 15399-C4, | | | | | | | | |
| 15399-D4, | | | | | | | | |
| 15399-E4, | | | | | | | | |
| 15399-F4, | | | | | | | | |
| 15399-G4, | | | | | | | | |
| 15399-H4, | | | | | | | | |
| 15399-A5 | | | | | | | | |
| 15399-B6, | L-Glu | | D-Leu | L-Val | L-Asp | D-Leu | L-Leu | 6 |
| 15399-E5, | | | | | | | | |
| 15399-G5, | | | | | | | | |
| 15399-F5, | | | | | | | | |
| 15399-C6, | | | | | | | | |
| 15399-C7, | | | | | | | | |
| 15399-F7, | | | | | | | | |
| 15399-A6, | | | | | | | | |
| 15399-H5, | | | | | | | | |
| 15399-D8, | | | | | | | | |
| 15399-G6 | | | | | | | | |
| 23960_A1 | L-Glu | | | | | | | 1 |

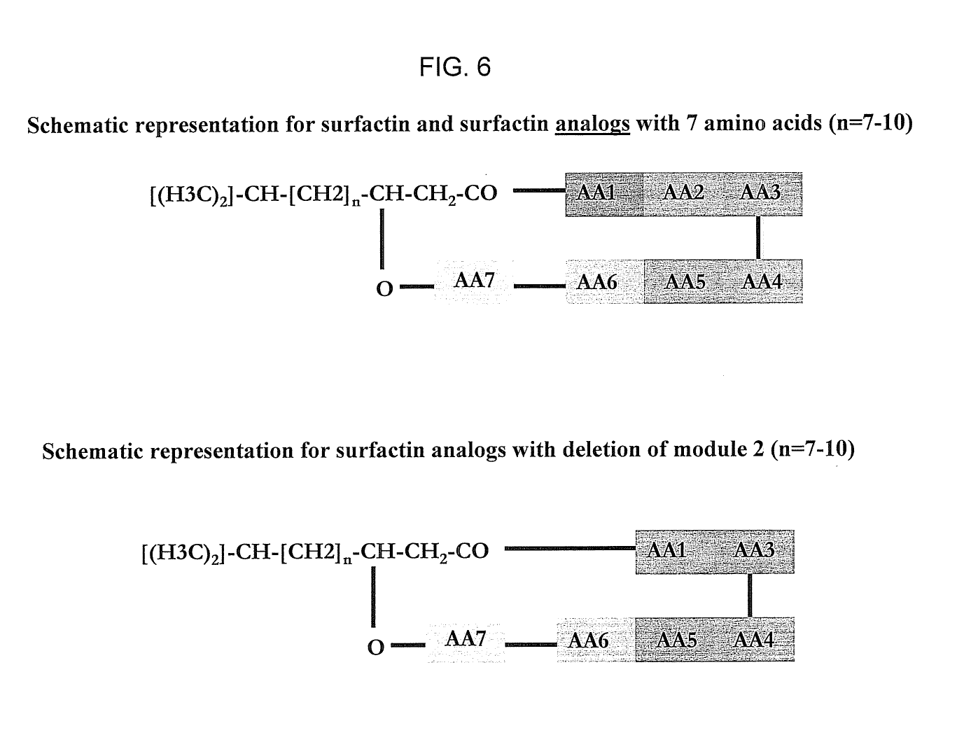

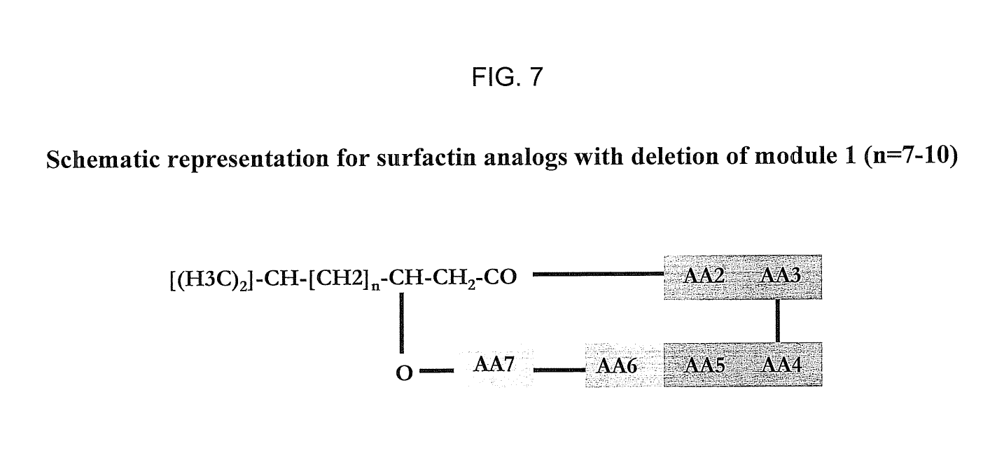

FIG. 6 and FIG. 7 are schematic representations of the structure of surfactin and surfactin analogs described herein.

### Example 4

### Engineering Lipopeptide Synthetase Polypeptides with a Deletion of a Peptide Synthetase Domain

The goal of example 4 was to join the highly variable sequences in the condensation domains of modules 1 and 3 of the first surfactin synthetase to engineer a 6-member (surfactin-analog) ring lacking the amino acid L-leucine, which is encoded by module 2 of the naturally occurring synthetase. Because of the large number of potential joining locations, we decided to establish a protocol that could easily be automated to generate multiple candidates. To that effect, we replaced the approximate region of chromosomal DNA to be deleted with a construct containing a “upp-kanamycin” cassette. In this construct, the cassette was flanked by sequence homologous to the DNA upstream of the variable region of condensation domain of module 2 and sequence homologous to the DNA downstream of the variable region of condensation domain of module 3. Deletions were established by joining the 3′-end of an approximately 1.3 kb region of the variable region of condensation domain of module 2 cassette and the 5′-end of an approximately 1.3 kb region of the variable region of condensation domain of module 3 in pUC19. Then, by site-directed deletions at the junction of the variable condensations domain regions, 11 plasmids were engineered to establish various boundaries between these regions. These plasmids were separately transformed into Bacillus subtilis competent cells.

Several colonies were picked following 18 hr incubation at 37° C. or 36 hr at 30° C. and grown in liquid media (LB with 25 μg/ml thymine and 100 μg/ml spectinomycin). Then, small aliquots of these cells were replica-plated on LB with 25 μg/ml thymine and 100 μg/ml spectinomycin, and LB with 30 μg/ml kanamycin. Cells that grew in the first plate but not in the one containing kanamycin were sequenced, since in those, likely, a recombination event replaced the “upp-kanamycin” cassette with the plasmid carrying the engineered boundaries between variable condensation domains of modules 2 and 3. The efficiency of selecting colonies by replica plating varied between 5-30%. Successful Bacillus subtilis constructs were grown in LB with 25 μg/ml thymine and the small molecules that were produced and secreted to the media were analyzed by MALDI.

Due to the high similarity that exists among modules, it was advantageous to do nested PCR reactions to amplify genomic DNA sequences. The template for the upstream flanking sequence of the upp-kan cassette was amplified from genomic DNA of OKB105 cells using primers VP-4C-sense-1: 5′-ATGCTGCTGTTTGACATGCACCA-3′ (SEQ ID NO:______) and VP-4D-anti-1:5′-CACCAGCTTGGCTCCGTTTAACA-3′ (SEQ ID NO:______).

This PCR product was named 4CD and was used to generate the fragment that was used to ligate to the upp-kan cassette using primers VP-4C-sense-1: 5′-ATGCTGCTGTTTGACATGCACCA-3′ (SEQ ID NO:______), and 4CD-BSTXI-BK: 5′-ATGTGCTACCACTCCTCTGGAGGCAGTGCTAAATTTCGCGCATCGGCATG-3′ (SEQ ID NO:______).

The upp-kan fragment was obtained from pUC19-UPP-KAN using primers 3-4-UPP-KAN-BSTXI-FW: 5′-ATGCTAAGCCAGAGGAGTGGGTTTTTTGACGATGTTCTTGAAACTCAATG-3′ (SEQ ID NO:______), and 3-4-UPP-KAN-BSTXI-BK: 5′-ATGTGCTACCAACTTCCTGGCAGAGTATGGACAGTTGCGGATGTACTTCAG-3′ (SEQ ID NO:______).

The downstream template for the flanking sequence of the upp-kan cassette was amplified from genomic DNA of OKB105 cells using primers VP-4H-sense-1: 5′-CGGAAATGTTCAGGTTCAGCGTG-3′ (SEQ ID NO:______) and VP-4G-anti-1: 5′-ATCGTCGGGTGCTGGTTGAGATC-3′ (SEQ ID NO:______).

This PCR product was named 4HG and was used to generate the fragment that was used to ligate to the upp-kan cassette using primers 4HG-BSTXI-FW-2: 5′-ATATCTGTCCAGGAAGTTGGATTCTTCTCGACGGATCACGCACGATTCTAAGC-3′ (SEQ ID NO:______), and VP-4G-anti-1: 5′-ATCGTCGGGTGCTGGTTGAGATC-3′ (SEQ ID NO:______).

All three fragments were separately digested with BstXI and ligated in a one step reaction. The ligation mixture was cleaned using Qiagen's PCR purification kit and transformed into OKB 105 Δupp Spect^{R} cells and plated on LB containing 30 μg/ml kanamycin plates. Colonies were screened by PCR-mapping and sequencing. The “upp-kan” marked strain was named OKB105 Δupp Spect^{R} upp⁺ kan^{R} (Proj4).

Plasmid Used to Generate Deletions

Deletions were established by joining the 3′-end of an approximately 1.3 kb region of the variable region of condensation domain of module 2 cassette and the 5′-end of an approximately 1.3 kb region of the variable region of condensation domain of module 3 in pUC19.

The template for generating the upstream flanking sequence was obtained by using 4CD as a template with primers 4CD-FW: 5′-CAGCTTCCTGATCTTCGTCTmCCAGTATAAGGACTACGCTGTATGGCAAAGC-3′ (SEQ ID NO:______), and 4CD-BK: 5′-AGGTAAAGCCAAATTTCGCGmCATCGGCATGATAGCTTTCTTCCTCATCG-3′ (SEQ ID NO:______), and cloning this fragment into pUC19 that was opened using primers pUC19 sense-8: 5′-GCGCGAAATTTGGCTTTACCmUAGCAGTGAATTAGAGCTCGGTACC-3′ (SEQ ID NO:______), and pUC19 anti-8: 5′-GAGACGAAGATCAGGAAGCTmGACCCAACTTAATCGCCTTGCAGCACATC-3′ (SEQ ID NO:______).

Both fragments were annealed and transformed into Sure cells and plated on CG-Amp. The resulting plasmid was named pUC19-4CD.

The template for generating the downstream flanking sequence was obtained by using 4HG as a template with primers 4HG-FW: 5′-GCGCGAAATTTGGCTTTACCmUATTCTTCTCGACGGATCACGCACGATTCTAAGC-3′ (SEQ ID NO:______), and 4HG-BK: 5′-ATGTAATCCGGCAGGGTTTCmCTTCAGTGCTGATTTCAGTGCTTCTATGTC-3′ (SEQ ID NO:______).

This PCR product was cloned into plasmid pUC19-4CD that was opened using primers pUC19-sense-9: 5′-GGAAACCCTGCCGGATTACAmUTTACGCTTACTCGCTCACTGACTCG-3′ (SEQ ID NO:______), and pUC19-anti-9: 5′-AGGTAAAGCCAAATTTCGCGmCATCGGCATGATAGCTTTCTTCCTCATCG-3′ (SEQ ID NO:______).

Both fragments were annealed and transformed into Sure cells and plated on CG-Amp. The resulting plasmid was named pUC19-4CD-4HG.

This plasmid was then used as a template to generate 11 fusion points between the 3′-end of 4CD and the 5′-end of 4HG. Each fusion point was engineered using pairs of primers listed below.

2082-1:B6:
(SEQ ID NO: )
5′-GAAATTTGGCTTTAAATCCTGmAAAATCCAGAAACAGCTGTAACCGCG-3′
2082-2:B6:
(SEQ ID NO: )
5′-TCAGGATTTAAAGCCAAATTTmCGCGCATCGGCATGATAGCTTTCTT-3′
2082-1:E5:
(SEQ ID NO: )
5′-GGCTTTACGCACGATTCTAAmGCCTGCCGCTTGATGAAAACGACGA-3′
2082-2:E5:
(SEQ ID NO: )
5′-CTTAGAATCGTGCGTAAAGCmCAAATTTCGCGCATCGGCATGATAG-3′
2082-1:G5:
(SEQ ID NO: )
5′-GCTTTACCGCTTGATGAAAAmCGACGAGGAGAATCCTGAAAATCCAGA-3′
2082-2:G5:
(SEQ ID NO: )
5′-GTTTTCATCAAGCGGTAAAGmCCAAATTTCGCGCATCGGCATGATA-3′
2082-1:F5:
(SEQ ID NO: )
5′-GAAATTTGGCTTTAAGCCTGmCCGCTTGATGAAAACGACGAGGAGA-3′
2082-2:F5:
(SEQ ID NO: )
5′-GCAGGCTTAAAGCCAAATTTmCGCGCATCGGCATGATAGCTTTCTT-3′
2082-1:C6:
(SEQ ID NO: )
5′-CGAAATTTGGCTTTAGAAAATmCCAGAAACAGCTGTAACCGCGGAGA-3′
2082-2:C6:
(SEQ ID NO: )
5′-GATTTTCTAAAGCCAAATTTCmGCGCATCGGCATGATAGCTTTCTTC-3′
2082-1:C7:
(SEQ ID NO: )
5′-GAAATGAAAATCCAGAAACAGmCTGTAACCGCGGAGAACTTGGCGTA-3′
2082-2:C7:
(SEQ ID NO: )
5′-GCTGTTTCTGGATTTTCATTTmCGCGCATCGGCATGATAGCTTTCTT-3′
2082-1:F7:
(SEQ ID NO: )
5′-GATGCGCGCACGATTCTAAGmCCTGCCGCTTGATGAAAACGACGAG-3′
2082-2:F7:
(SEQ ID NO: )
5′-GCTTAGAATCGTGCGCGCATmCGGCATGATAGCTTTCTTCCTCATCG-3′
2082-1:A6:
(SEQ ID NO: )
5′-GAAATTTGGCTTTAGAGGAGAAmUCCTGAAAATCCAGAAACAGCTGTAACC-3′
2082-2:A6:
(SEQ ID NO: )
5′-ATTCTCCTCTAAAGCCAAATTTmCGCGCATCGGCATGATAGCTTTCTT-3′
2082-1:H5:
(SEQ ID NO: )
5′-AATTTGGCTTTAGATGAAAACmGACGAGGAGAATCCTGAAAATCCAGAAA-3′
2082-2:H5:
(SEQ ID NO: )
5′-CGTTTTCATCTAAAGCCAAATmUTCGCGCATCGGCATGATAGCTTTC-3′
2082-1:D8:
(SEQ ID NO: )
5′-ATGCGGAAAATCCAGAAACAmGCTGTAACCGCGGAGAACTTGGCGTA-3′
2082-2:D8:
(SEQ ID NO: )
5′-CTGTTTCTGGATTTTCCGCAmUCGGCATGATAGCTTTCTTCCTCATC-3′
2082-1:G6:
(SEQ ID NO: )
5′-CGAAATCCGCTTGATGAAAAmCGACGAGGAGAATCCTGAAAATCCAGA-3′
2082-2:G6:
(SEQ ID NO: )
5′-GTTTTCATCAAGCGGATTTCmGCGCATCGGCATGATAGCTTTCTTC-3′

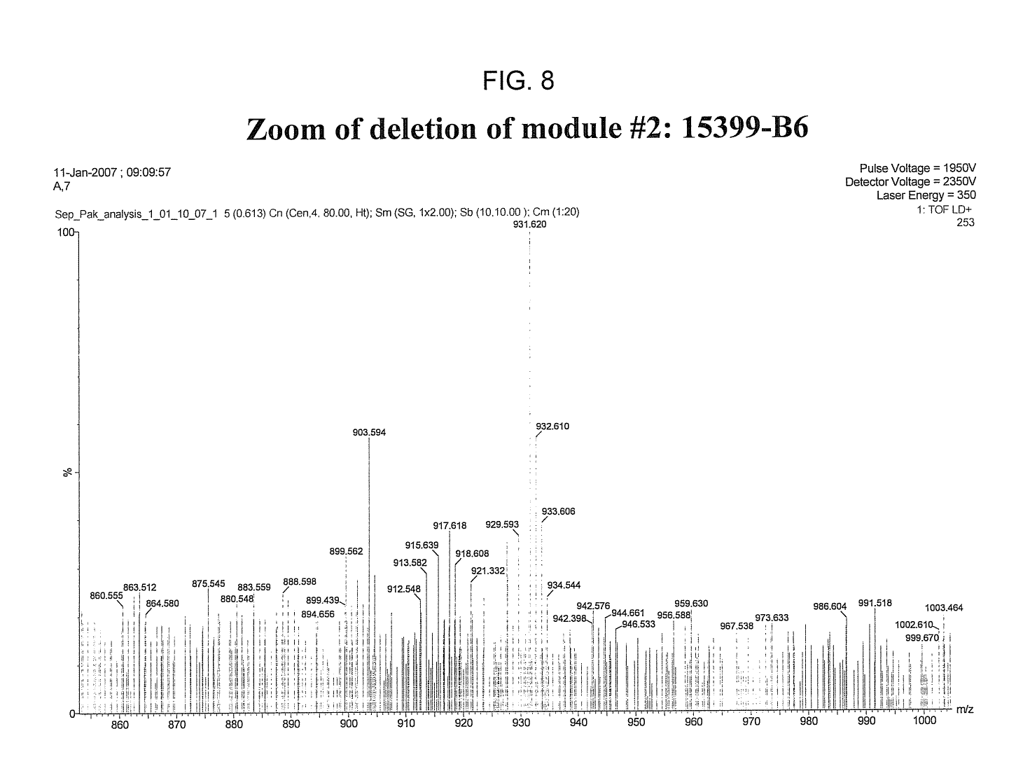

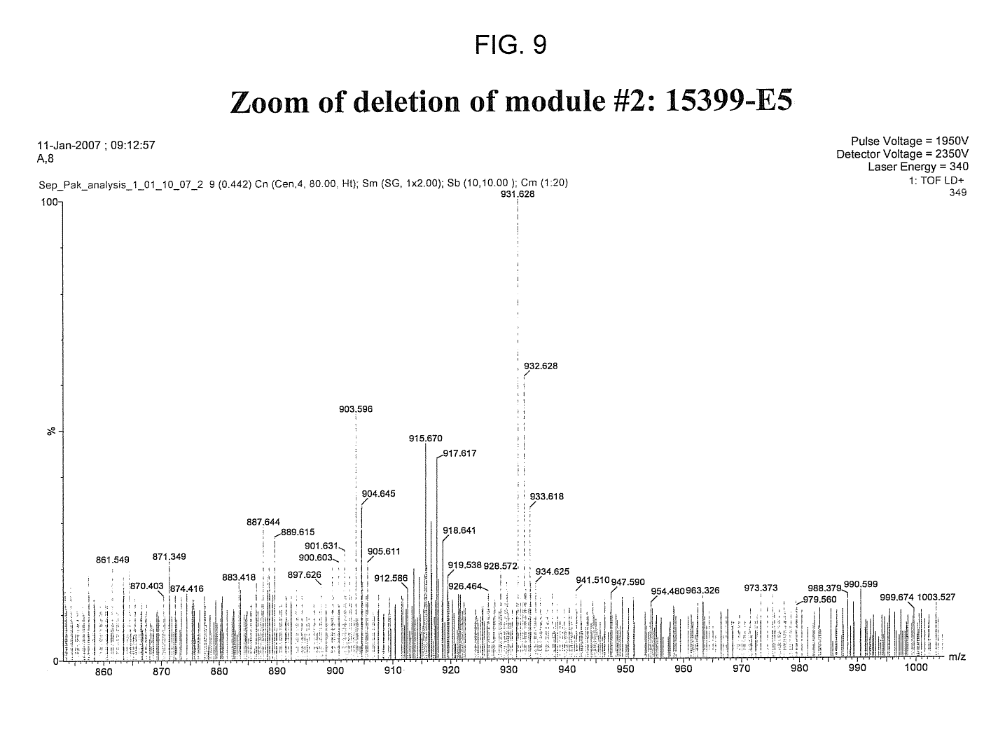

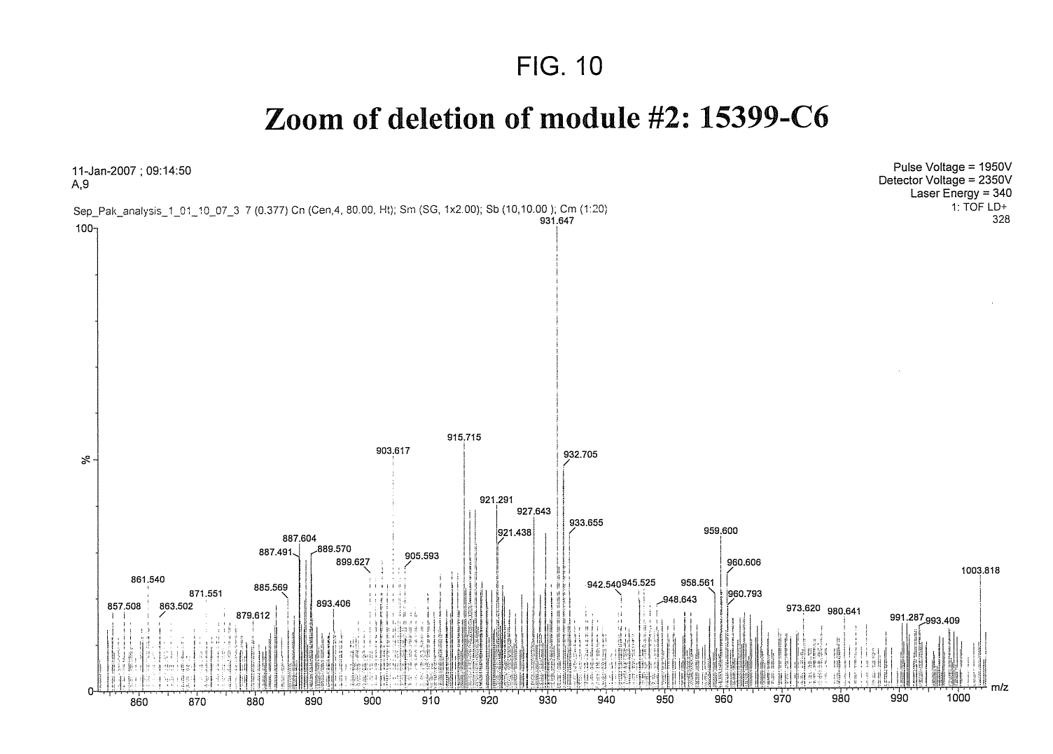

The annealed fragments were transformed into Sure cells and selected on CG-Amp. The resulting plasmids were named pUC19-Del-Mod2-B6, pUC19-Del-Mod2-E5, pUC19-Del-Mod2-G5, pUC19-Del-Mod2-F5, pUC19-Del-Mod2-C6, pUC19-Del-Mod2-C7, pUC19-Del-Mod2-F7, pUC19-Del-Mod2-A6, pUC19-Del-Mod2-H5, pUC19-Del-Mod2-D8, pUC19-Del-Mod2-G6. These plasmids were transformed into OKB105 Δupp Spect^{R} upp⁺ kan^{R} (Proj4) to yield strains 15399-B6, 15399-E5, 15399-G5, 15399-F5, 15399-C6, 15399-C7, 15399-F7, 15399-A6, 15399-H5, 15399-D8, 15399-G6. MALDI analysis of compounds produced by three of the strains is shown in FIG. 8, FIG. 9, and FIG. 10.

### Example 5

### Engineering a Lipopeptide Synthetase Polypeptide to Include a Heterologous Module

We engineered a recombinant lipopeptide synthetase polypeptide which includes peptide synthetase domains (modules) of surfactin synthetase. In this engineered polypeptide, module 2 of surfactin synthetase, which encodes L-Leu, was replaced with other modules using the homology that exists among modules in the thiolation domain. In particular, we chose the L-Tyr and L-Phe modules from tyrocidine, and L-Leu from linear gramicidin.

Due to the high similarity that exists among modules, it was advantageous to perform nested PCR reactions to amplify genomic DNA sequences. The upstream flanking sequence of the upp-kan cassette was amplified using pUC19-2CD as the template (see Example 2) using primers: 1CD-and-2CD-FW: 5′-GGATGTGCTGCAAGGCGATTAAGTTGGGTCTG-3′ (SEQ ID NO:______), and 5-2CD-BstXI-BK: 5′-ATGCTAATCCACTCCTCTGGGTCAAAGATACCAGCCTTCTCAACG-3′ (SEQ ID NO:______).

The upp-kan fragment was obtained from pUC19-UPP-KAN using primers 3-4-UPP-KAN-BSTXI-FW: 5′-ATGCTAAGCCAGAGGAGTGGGTTTTTTGACGATGTTCTTGAAACTCAATG-3′ (SEQ ID NO:______), and 3-4-UPP-KAN-BSTXI-BK: 5′-ATGTGCTACCAACTTCCTGGCAGAGTATGGACAGTTGCGGATGTACTTCAG-3′ (SEQ ID NO:______).

The downstream template for the flanking sequence of the upp-kan cassette was amplified from pUC-2CD-45DR-2HG using the primers 5-2HG-BstXI-FW: 5′-ATTACTACCCAGGAAGTTGGCACTTCTTTGACATTGGAGGACATTCATTAGCAGG-3′ (SEQ ID NO:______), and 1HG-and-2HG-BK: 5′-TGATTCTGTGGATAACCGTATTACCGCCTTTGAGTG-3′ (SEQ ID NO:______).

All three fragments were separately digested with BstXI and ligated in a one step reaction. The ligation mixture was cleaned using Qiagen's PCR purification kit and transformed into OKB 105 Δupp Spect^{R} cells and plated on LB containing 30 μg/ml kanamycin plates. Colonies were screened by PCR-mapping and sequencing. The “upp-kan” marked strain was named OKB105 Δupp Spect^{R} upp⁺ kan^{R} (Proj5).

The insert that encodes L-Tyr was obtained using nested-PCR. The initial set of primers that was used to amplify this module was obtained from the genomic DNA of strain ATCC8185 using primers 015252:5′-AAGCTCGCAGCGATATGGGAA-3′ (SEQ ID NO:______), and 015316:5′-AACGCCTTGATCGTAGGCTGC-3′ (SEQ ID NO:______).

The resulting product was used for PCR amplification using primers 015537: 5′-GAGAAAGCAGGAATCTTTGAmCCATTTCTTTGAACTGGGCGGA-3′ (SEQ ID NO:______), and 015538: 5′-TCCTCCAATGTCAAAGAAGTmGGTCGAGAATGCCGATGCCG-3′ (SEQ ID NO:______).

The resulting fragment was annealed to pUC19-2CD-45DR-2HG that was opened with pUC19-ps-ins-1-sense: 5′-CACTTCTTTGACATTGGAGGmACATTCTTTAGCTGGTATGAAGATGCC-3′ (SEQ ID NO:______), and pUC19-ps-ins-2-anti: 5′-GTCAAAGATTCCTGCTTTCTmCAACGTTCAGCACGTCCTGCC-3′ (SEQ ID NO:______).

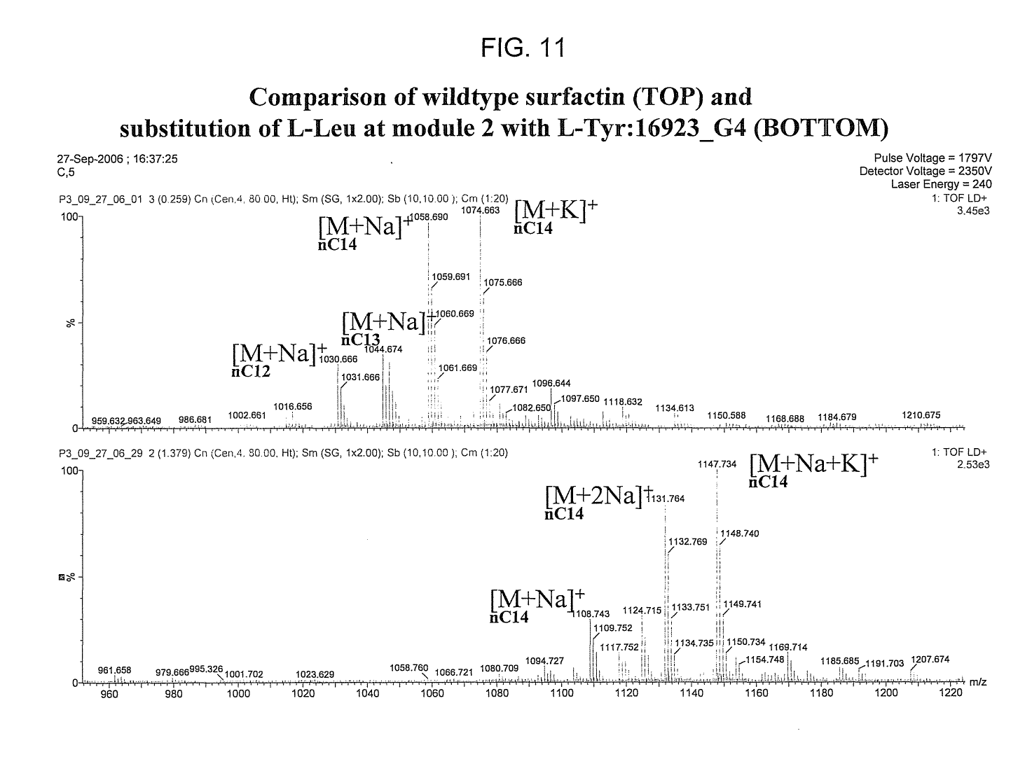

The resulting plasmid was named pUC19-L-Tyr-mod2 and was transformed into OKB105 Δupp Spect^{R} upp⁺ kan^{R} (Proj5). The resulting strain with the desired mutation was named 16923 G4. (see slides 56-60). Production of the expected small molecule was good (see Table 7). A comparison of MALDI analysis of compounds produced by strain 16923_G4 and a strain that produces wild type surfactin is shown in FIG. 11.

The insert that encodes L-Phe was obtained using nested-PCR. The initial set of primers that was used to amplify this module was obtained from the genomic DNA of strain ATCC8185 using primers 015232: 5′-TTGGGAGCAAATTCTTGGCGT-3′ (SEQ ID NO:______), and 015296:5′-TGAAACTCGCGATGCACTTGC-3′ (SEQ ID NO:______).

The resulting product was used for PCR amplification using primers 015529: 5′-GAGAAAGCAGGAATCTTTGAmCCATTTTTTCACGCTGGGCG-3′ (SEQ ID NO:______), and 015530:5′-TCCTCCAATGTCAAAGAAGTmGATCCAACACCCCGACGCC-3′ (SEQ ID NO:______).

The resulting fragment was annealed to pUC19-2CD-45DR-2HG that was opened with pUC19-ps-ins-1-sense: 5′-CACTTCTTTGACATTGGAGGmACATTCTTTAGCTGGTATGAAGATGCC-3′ (SEQ ID NO:______), pUC19-ps-ins-2-anti: 5′-GTCAAAGATTCCTGCTTTCTmCAACGTTCAGCACGTCCTGCC-3′ (SEQ ID NO:______).

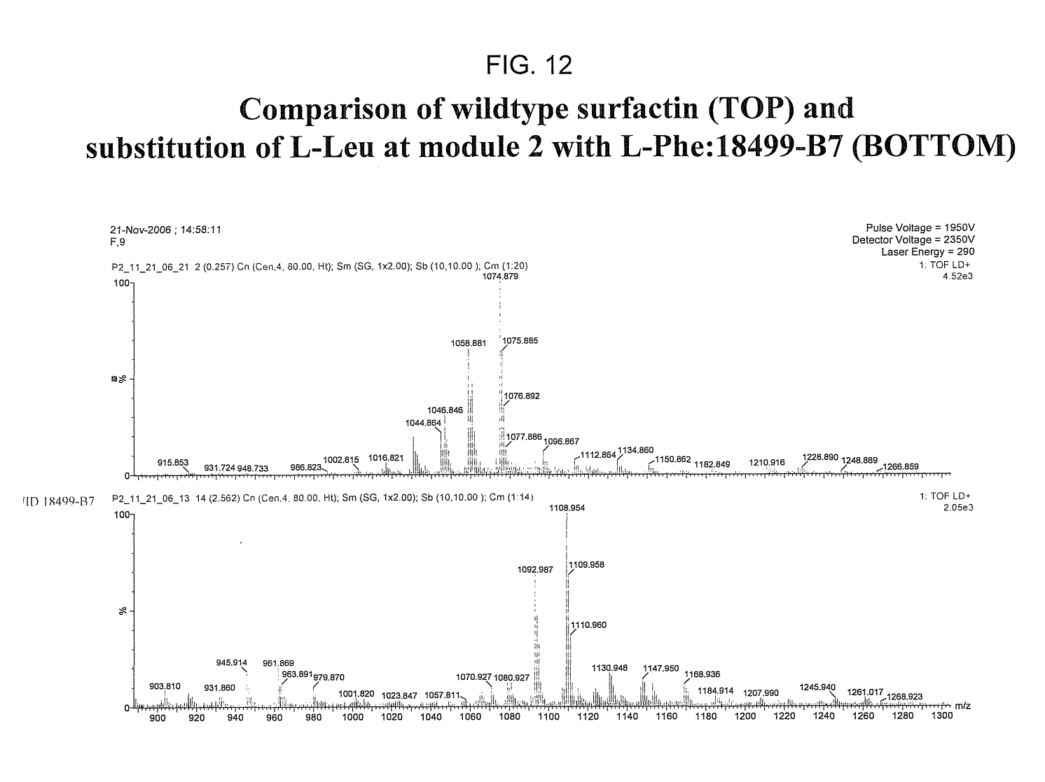

The resulting plasmid was named pUC19-L-Phe-mod2 and was transformed into OKB105 Δupp Spect^{R} upp⁺ kan^{R} (Proj5). The resulting strains with the desired mutations were named 18499 B7. A comparison of MALDI analysis of compounds produced by strain 18499_B7 and a strain that produces wild type surfactin is shown in FIG. 12. Production of the expected small molecule was low (see Table 7).

The insert that encodes L-Leu was obtained using nested-PCR. The initial set of primers that was used to amplify this module was obtained from the genomic DNA of strain ATCC8185 using primers 015245: 5′-CGACGGAGGAAATGGTAGCGA-3′ (SEQ ID NO:______), and 015309:5′-CGGGACACGATCTGGATGCTC-3′ (SEQ ID NO:______).

The resulting product was used for PCR amplification using primers 015515: 5′-GAGAAAGCAGGAATCTTTGAmCGATTTCTTTGAGCGGGGCG-3′ (SEQ ID NO:______), and 015516: 5′-TCCTCCAATGTCAAAGAAGTmGATCGTGTATCCCAACATCCGC-3′ (SEQ ID NO:______).

The resulting fragment was annealed to pUC19-2CD-45DR-2HG that was opened with pUC19-ps-ins-1-sense: 5′-CACTTCTTTGACATTGGAGGmACATTCTTTAGCTGGTATGAAGATGCC-3′ (SEQ ID NO:______), and pUC19-ps-ins-2-anti: 5′-GTCAAAGATTCCTGCTTTCTmCAACGTTCAGCACGTCCTGCC-3′ (SEQ ID NO:______).

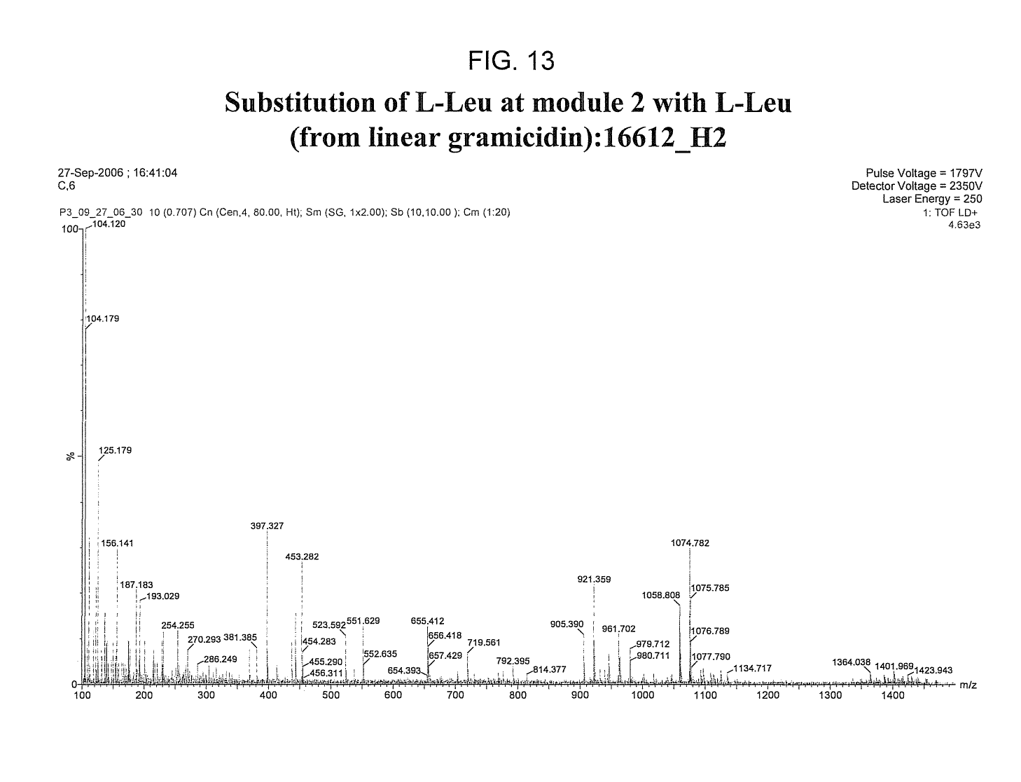

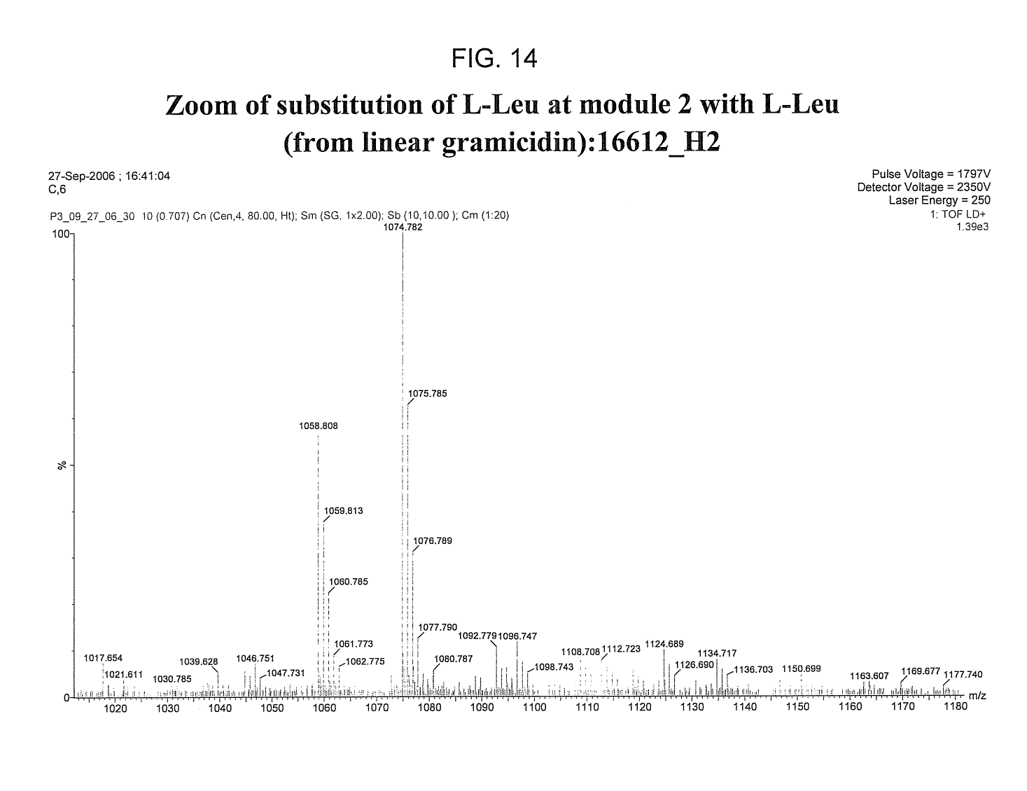

The resulting plasmid was named pUC19-L-Leu-mod2 and was transformed into OKB105 Δupp Spect^{R} upp⁺ kan^{R} (Proj5). The resulting strains with the desired mutations were named 16612_H2. MALDI analyses of compounds produced by this strain are shown in FIG. 13 and FIG. 14. The production of wildtype surfactin in strain 16612_H2 was lower than that produced in OKB105 Δupp Spect^{R} (see Table 7).

### Example 6

### Engineering a Surfactin Synthetase Polypeptide that Produces a Lipo-di-peptide (Fatty Acid-Glu-Leu)

In this example, we engineered a polypeptide that would produce a surfactin analog in which the last five amino acids of surfactin were deleted. In particular, the construct produced the molecule FA-Glu-Leu, where “FA” encodes the variable length fatty acid that is present in wildtype surfactin, and “Glu” and “Leu” correspond to “glutamic acid” and “leucine”, the first two amino acids that are present in wildtype surfactin.

The construct encoding the engineered polypeptide involved seamless in-frame fusion of the thioesterase domain present at the 3′-end of the SrfA-C to the 3′-end of module 2 of SrfA-A. The construct used a fusion point located upstream of the consensus sequence GGHSL and a starting strain in which the competence gene ComS was under the regulation of the surfactin promoter at the AmyE locus of surfactin. This gene is always present in strains lacking module 4 of surfactin synthetase, because this gene is present out-of-frame, with respect to genes in the second synthetase of surfactin, in module 4 under the regulation of the surfactin promoter.

This construct was engineered by starting with the plasmid named pUC19-KAN-DR-ASP-TE, which was designed for the construction of FA-Glu-Asp-TE-MG (see Example 7, below). In this plasmid, DR refers to the DNA sequence, which is identical to the 3′-end of the module that encodes glutamic acid.

To obtain a seamless fusion containing the second module of surfactin,the plasmid pUC19-KAN-DR-ASP-TE was opened using primers 020405: 5′-GTCAAAGATCCCCGCCTTCTmCAACGTTCAGCACGTCCT-3′ (SEQ ID NO:______), and 020406: 5′-GATTTCTTTGCGCTCGGAGmGGCATTCCTTGAAGGCC-3′(SEQ ID NO:______), and an insert was obtained by PCR amplification of total genomic DNA of strain OKB105 using primers_{—}020407: 5′-GAGAAGGCGGGGATCTTTGAmCAATTTCTTTGAAACTGGCGGACATTCATTAA-3′ (SEQ ID NO:______), and 020408: 5′-CCTCCGAGCGCAAAGAAATmCGTCATAAGCGCCGACTTGTTCT-3′ (SEQ ID NO:______).

The resulting plasmid and insert were annealed and transformed into Sure cells. A plasmid with the desired sequence was named pUC19-KAN-DR-LEU-TE-MG. This plasmid was subsequently transformed into OKB105 Δ(upp)Spect^{R}(PsurfComS)(Δmod(2-7)) upp⁺ Kan^{R}. As a result of this transformation, there was a double crossover event between plasmid and chromosomal KAN and TE sequences. Then, the DR sequence recombined with chromosomal GLU sequences leading to the excision of “upp-kan”. The resulting Bacillus strain was named OKB105Δ(upp)Spect^{R}(P_{surf}ComS)(GLU-LEU-TE). Candidate transformants were replica plated on LB-spectinomycin (100 ug/ml) -thymine(25 ug/ml) and LB-kanamycin (30 ug/ml). Selected constructs were grown in 1 ml of M9YE containing 1% casamino acids and 0.5% glucose for 5 days at 30° C. in 2.2 ml microtiter plates. Following growth, 450 ul of M9YE was added and plates were spun at 3.5kg for 20 minutes to separate cell mass from supernatant. A total of 750 ul of supernatant was recovered and acidified with 250 ul of water containing 12 ul of concentrated HCl. Following incubation on ice for 2 hrs, plates were centrifuged at 3.5 kg for 5 minutes and pellets were resuspended in 500 ul of 100% methanol with shaking Soluble material was analyzed by MALDI in positive mode. Once a perfect clone was identified, it was grown either in M9YE containing 1% casamino acids and 0.5% glucose or in M9 salts+corn steep liquor (0.3% protein content)+0.5% glycerol.

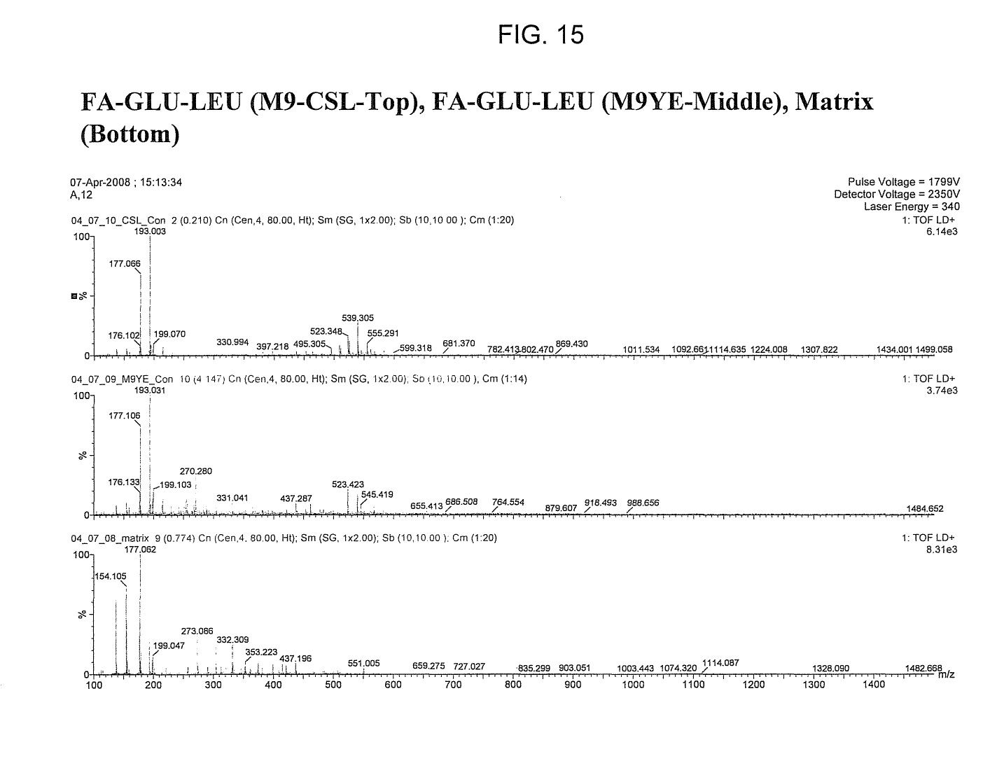

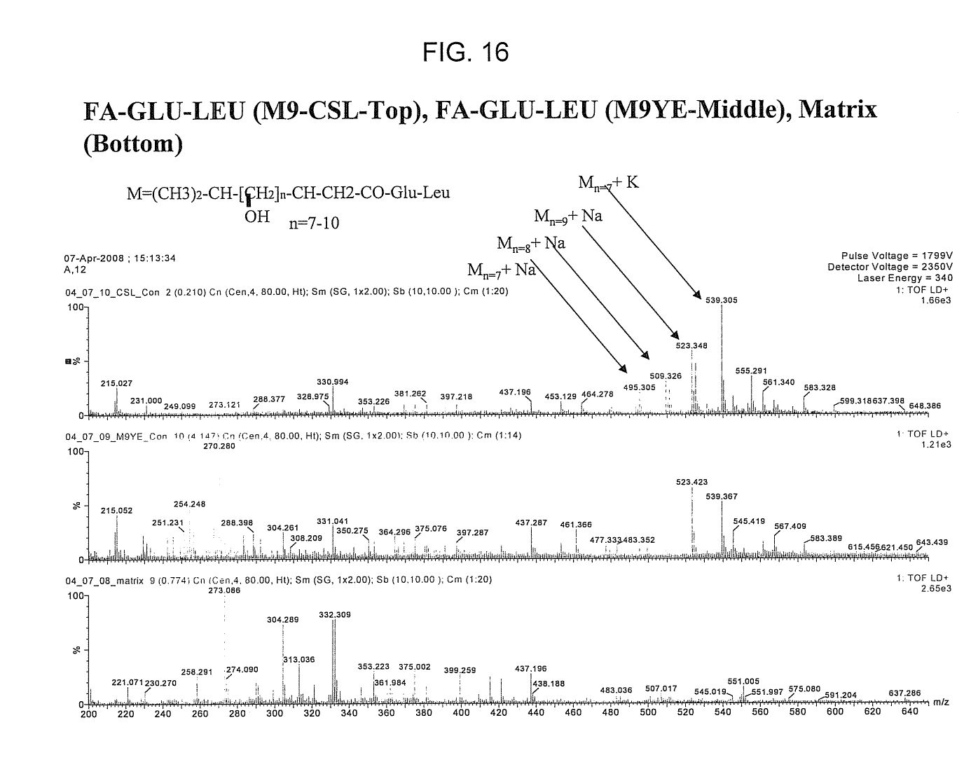

MALDI spectra, indicating the product FA-GLU-LEU, are shown in FIG. 15 and FIG. 16.

### Example 7

### Engineering a Surfactin Synthetase Polypeptide that Produces a Lipo-di-Peptide (Fatty Acid-Glu-Asp)

We decided to test if we could engineer a construct that would contain a fatty acid followed by glutamic acid covalently linked to aspartic acid using as a fusion point a region located upstream of the consensus sequence GGHSL.

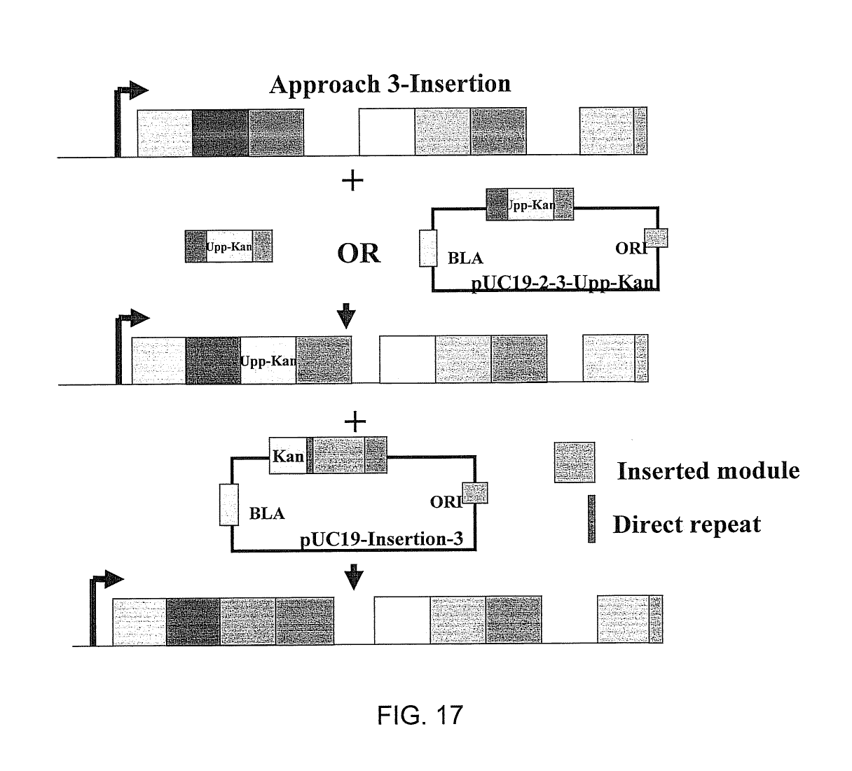

The starting strain that was used for the synthesis of FA-GLU-ASP was OKB105 Δ(upp)Spect^{R}(Δmod(2-7)) upp⁻ Kan^{R}. The approach that was selected is illustrated in FIG.17, where the inserted module corresponds to the module that encodes Asp. Due to the high similarity that exists among surfactin modules, it was advantageous to do nested PCR reactions to amplify genomic DNA sequences. The first PCR to amplify a region of DNA encoding ASP was carried out using the outer primers 019129: 5′-ACTGAACATGGCTGAGCATGTG-3′ (SEQ ID NO:______) and 019130:5′-AAGCTCTCCTTCCATTAGAAGAACAG-3′ (SEQ ID NO:______).

Then, the PCR product was further amplified using primers 019133: 5′-GAGAAGGCGGGGATCTTTGAmCAACTTCTTTATGATCGGCGGCC-3′ (SEQ ID NO:______) and 019134:5′-CCTCCGAGCGCAAAGAAATmCGTCATCAATGCCGATGGCTTC-3′ (SEQ ID NO:______).

The resulting PCR product was annealed to the PCR product that resulted from amplifying pUC19-KAN-DR-TE with 019131:5′-GTCAAAGATCCCCGCCTTCTmCAACGTTCAGCACGTCCT-3′ (SEQ ID NO:______) and 019132:5′-GATTTCTTTGCGCTCGGAGmGGCATTCCTTGAAGGCC-3′ (the annealed mixture was transformed into Sure cells. The resulting plasmid was named pUC19-KAN-DR-ASP-TE. A partial sequence of this construct is given in (SEQ ID NO:______), shown below; (SEQ ID NO:______) does not show the 5′-end of the GLU module, which is wild type sequence that corresponds to nucleotide positions 1-1809 of wild type surfactin synthetase. This plasmid was subsequently transformed into OKB105 Δ(upp)Spect^{R} FA-GLU-ASP-TE-MG.

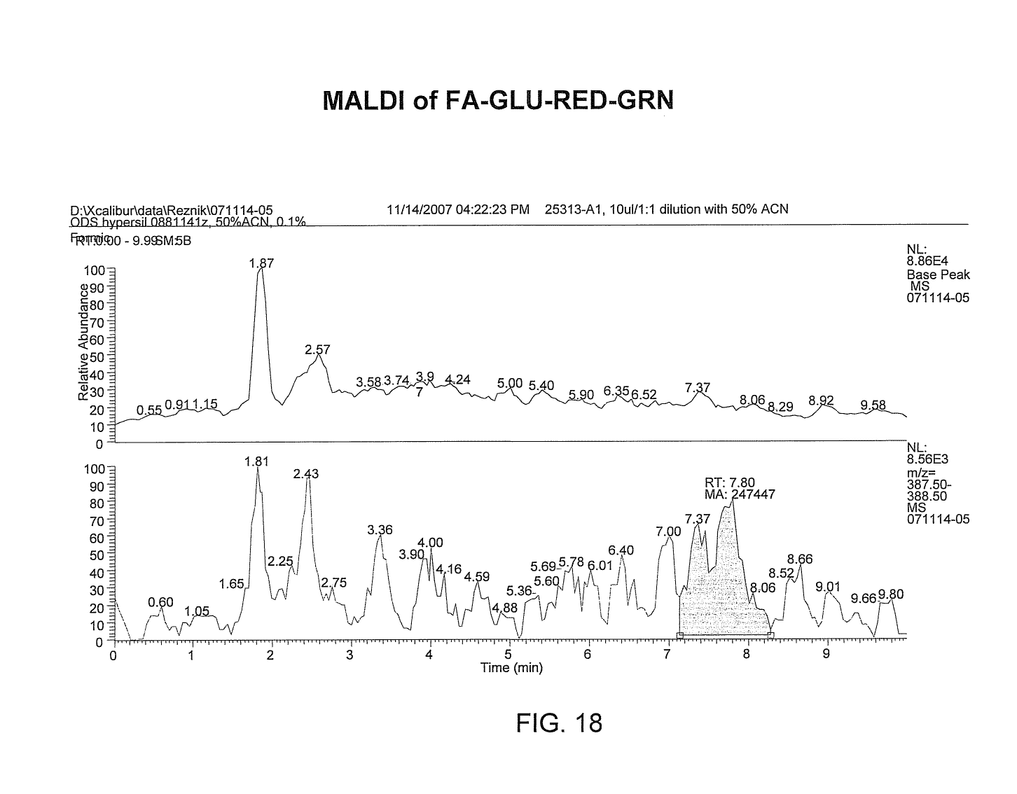

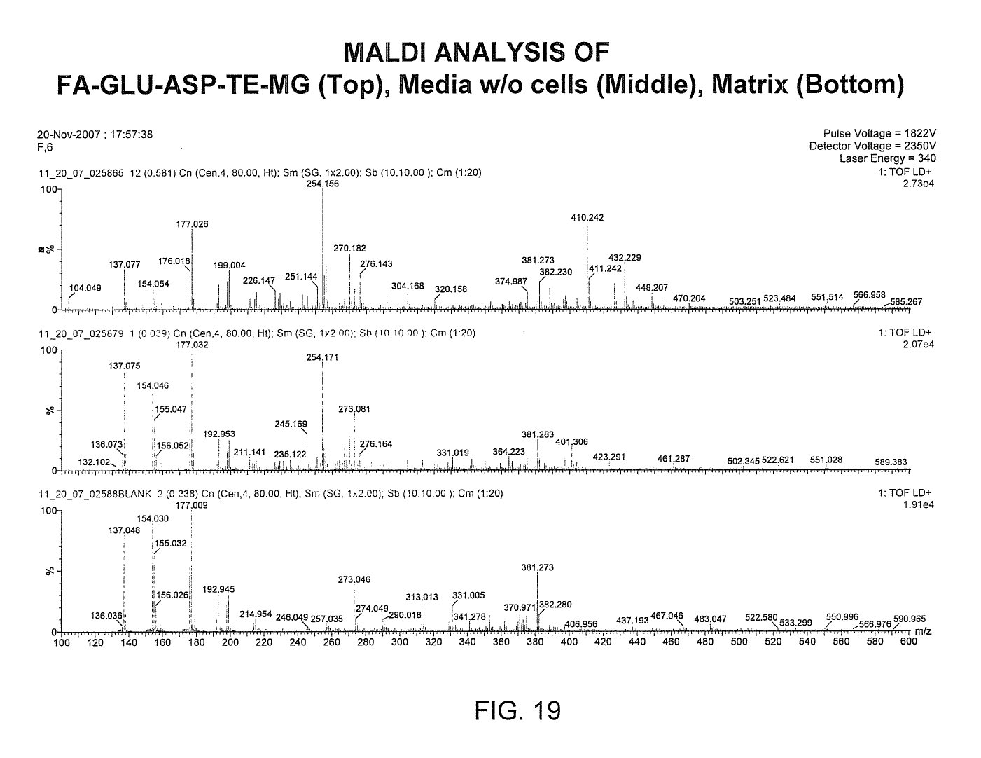

Cells were grown in M9YE+1% casamino acids and 0.5% glucose for 5 days at 30° C. The supernatant was passed through a C18 column, washed with 10% methanol and eluted with 100% methanol. The eluted material was concentrated and analyzed by MALDI (see FIG. 18). The resulting MS spectra revealed that the expected FA-GLU-ASP-TE was not visible. However, there were four large peaks corresponding to FA-GLU+Na, FA-GLU+K, FA-GLU+2Na, and FA-GLU+Na+K adducts, indicating that the thioesterase had nonspecifically cut the amide bond between the carboxyl group directly linked to the alpha carbon of glutamic acid and the amino group of aspartic acid. LC-MS quantitative analysis revealed that the titer of FA-GLU in the sample derived from the FA-GLU-ASP-TE-MG construct was 116.8 mg/l (see FIG. 19).

TABLE 9
Summary of titer results for the production of FA-GLU

| | FA-GLU-TE-MG | FA-GLU-ASP-TE-MG |
|---|---|---|
| Highest titer of | 9.67 mg/l | 116.8 mg/l |
| FA-GLU | | |

### Example 8

### Anaerobic Fermentation of Surfactin and Fatty Acid-Glu Acid-Leu

In this example, we tested the ability of the surfactin producing strain of Bacillus subtilis (strain OKB 105 Δ(upp)Spect^{R}) and the FA-Glu-Leu producing strain of Bacillus subtilis (27124-C1, strain OKB105 Δ(upp)Spect^{R} lacking modules 3-7 of wild-type surfactin synthetase) to grow in anaerobic media as described by Davis and Varley, Enzyme and Microbial Technology (1999) 25:322-329.

Anaerobic Media:

Anaerobic media (Media E) was derived from Davis and Varley, Enzyme and Microbial Technology 25 (1999) 322-329. Media E is composed of a base media, Wolin's trace metal solution, ammonium sulfate ((NH₄)₂SO₄), and magnesium sulfate (MgSO₄). The base media consists of (KH₂PO₄ (2.7 g/L), K₂HPO₄ (13.9 g/L), NaCl (50 g/L), sucrose (10 g/L), yeast extract (0.5 g/L), and NaNO₃ (1 g/L). NaNO₃ and (NH₄)₂SO₄ were omitted from Media E in this work, and replaced with 4 g/L NH₄NO₃, as suggested by Davis and Varley. Also, 0.5 g/L of NaCl was used for this work instead of the 50 g/L. Wolin's trace metals, as described by M. McInerney, was replaced by trace salts solution referenced in Davis and Varley and described by J. B. Clark, D. M. Munnecke, and G. E. Jenneman Dev. Ind. Microbiol. (1981) 22:695-701. The trace salts solution is composed of (g/L); EDTA, 1.0; MnSO₄, 3.0; FeSO₄, 0.1; CaCl₂, 0.1; CoCl₂, 0.1; ZnSO₄, 0.1; CuSO₄, 0.01; AlK(SO₄)₂, 0.01; H₃BO₄, 0.01; and Na₂MoO₄, 0.01. For these experiments, AlK(SO₄)₂ was omitted. In addition to the four components described by M. McInerney, Davis and Varley described the use of 40 g/L glucose and 0.1 g/L iron sulfate.

The base media, trace salts, ammonium sulfate, and magnesium sulfate solutions are made separately, autoclaved for sterility, and combined as follows; 970 mL base media, 10 mL trace metals, 10 mL ammonium sulfate, and 10 mL magnesium sulfate. Due to the addition of 80 mL of 500 g/L glucose and 10 mL of 10 g/L iron sulfate for this work, the base media volume was decrease to 880 mL. The glucose and iron sulfate solutions were filter sterilized prior to inclusion in the final Media E preparation.

To remove oxygen from the media and reaction vessels, N₂ gas was purged into the media for 1 hour (500 mL cultures) and 30 minutes (250 mL cultures). After N₂ purging, the culture conditions were assumed to be devoid of oxygen, thus making them anaerobic.

As a comparison for the low aeration and low stirring conditions, Bacillus subtilis (strain OKB105 Δ(upp)Spect^{R}) was grown in M9YE (6 g/L Na₂HPO₄, 3 g/L KH₂PO₄, 0.5 g/L NaCl, 1 g/L NH₄Cl) with 5 g/L glucose and 5 g/L casamino acids. The Bacillus subtilis (strain OKB105 Δ(upp)Spect^{R}) M9YE culture was grown without N₂ purging, and thus used to determine if low stirring could support adequate nutrient mixing.

Preparation of Inoculum and Culture Conditions:

Bacillus subtilis (strain OKB105 Δ(upp)Spect^{R}) and Bacillus subtilis 27124-C1 (strain OKB105 Δ(upp)Spect^{R} lacking modules 3-7 of wild-type surfactin synthetase) were streaked-out on LB agar media containing Thymine (25 μg/mL) and Spectinomycin (100 μg/mL). Strains were grown for 16-20 hours at 30° C. prior to the addition of a cell mass to the shake-flasks containing media. Strains were added prior to the purging with N₂ gas.

After the N₂ purging, the shake-flasks were placed in a 30° C. incubator and stirred gently to provide mixing. The anaerobic cultures were grown in a 30° C. incubator for 5 days prior to analysis of product formation.

Purification of Surfactin and FA-Glu-Leu from Fermentation Broth:

After 5 days of 30° C. incubation under anaerobic conditions, 500 μL of fermentation broth was centrifuged at 10,000×g for 10 minutes to generate cell free supernatant. The cell free supernatant was applied to a C18 column for solid-phase extraction of surfactin and FA-Glu-Leu. Both surfactin and FA-Glu-Leu were eluted from the C 18 column with 100% methanol, dried under vacuum, and resuspended in a 10-times concentrated volume of 100% methanol or 50% methanol:water for MALDI analysis.

Alternatives to C18 solid-phase purification of FA-Glu-Leu have been evaluated. Liquid-liquid extractions using the fermentation broth as liquid A, and a 1:1 volumetric ratio of the organic solvents ethyl acteate, butanol, hexane, or chloroform as liquid B all showed the capacity to extract FA-Glu-Leu from the fermentation broth. The method was as follows; after the collecting the organic phase from each liquid-liquid extraction, the organic solvent containing FA-Glu-Leu was dried under vacuum until a dry pellet was collected. The dry pellet was extracted first with 1/10^{th} volume of 100% methanol and then with 1/10^{th} volume of 100% water prior to MALDI analysis. The methanol and water extractions were cleaned via C18 purification to provide clean desalted samples for MALDI analysis.

FA-Glu-Leu could also be extracted from the fermentation broth using the hydrochloric acid to lower the pH of the fermentation broth to pH=2. At pH 2, the FA-Glu-Leu compound precipitated and was recovered using 1/10^{th} volume 100% methanol extraction of the acid precipitated pellet. The methanol and water extractions were cleaned via C18 purification to provide clean desalted samples for MALDI analysis.

MALDI Analysis of Surfactin from Fermentation Broth:

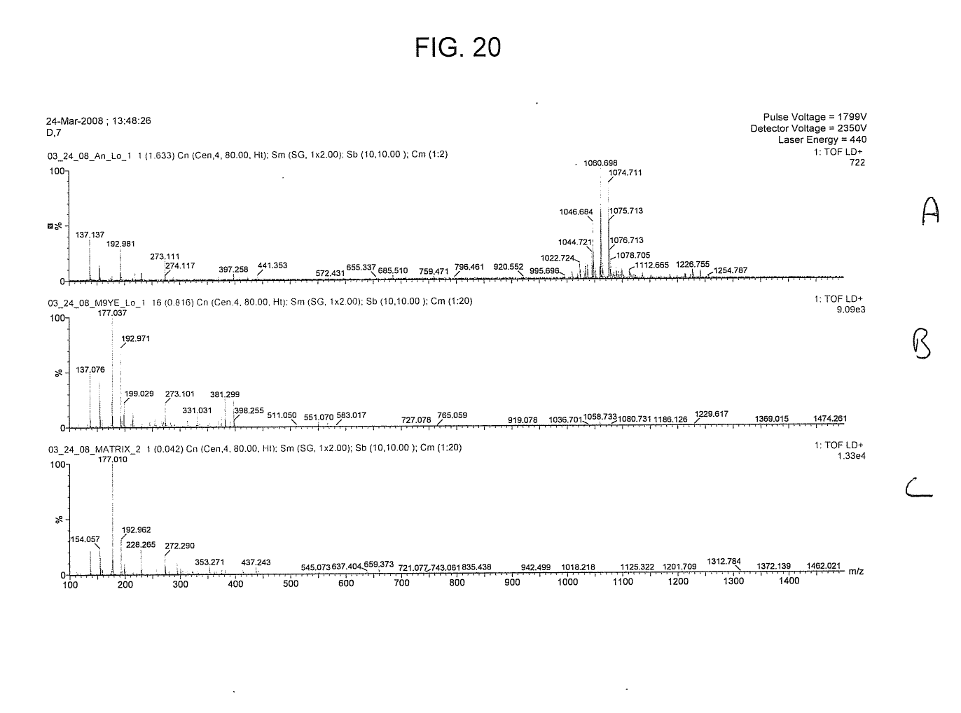

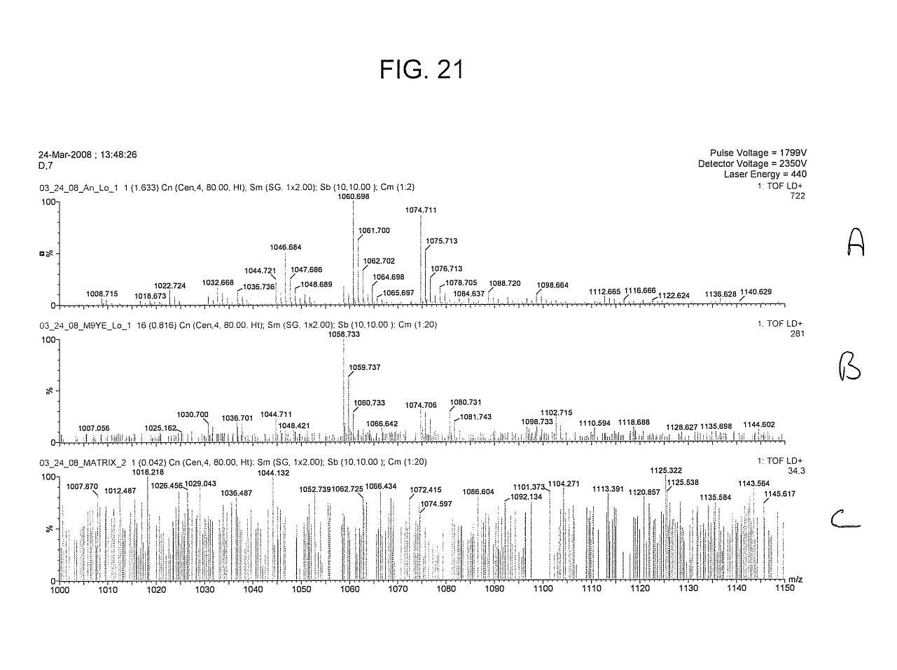

Surfactin was detected in the fermentation broth from the anaerobic culture after 5 days of incubation at 30° C. under anaerobic conditions; see FIG. 20(A). Surfactin was also detected in the fermentation broth for the M9YE culture grown under conditions of low aeration, see FIG. 21(B), but at an intensity much lower than that of the anaerobically grown culture.

MALDI Analysis of FA-Glu-Leu from Fermentation Broth:

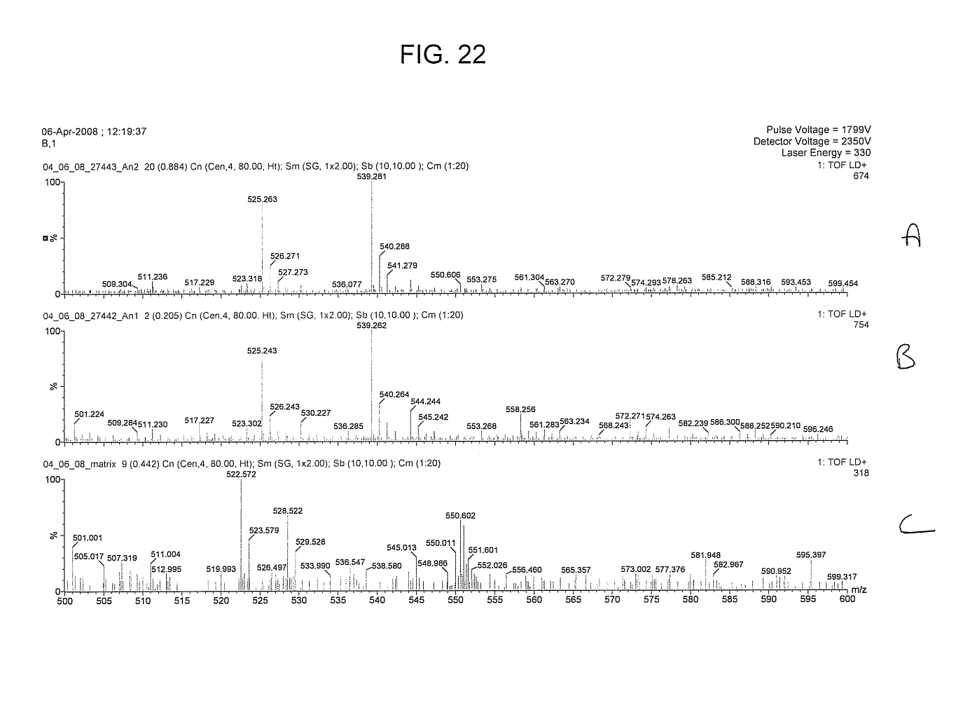

FA-Glu-Leu was detected in the fermentation broth from the anaerobic culture after 5 days of incubation at 30° C. under anaerobic conditions; see FIGS. 22(A and B). FA-Glu-Leu production under anaerobic conditions was not enhanced using twice the concentration of glucose (80 g/L glucose, FIG. 22A), or using twice the concentration of ammonium nitrate (8 g/L ammonium nitrate, FIG. 22B).

The foregoing description is to be understood as being representative only and is not intended to be limiting. Alternative methods and materials for implementing the invention and also additional applications will be apparent to one of skill in the art, and are intended to be included within the accompanying claims.

1. An engineered lipopeptide synthetase polypeptide which is a deletion mutant of a naturally occurring lipopeptide synthetase polypeptide,
wherein the naturally occurring polypeptide comprises a first and second peptide synthetase domain, wherein each peptide synthetase domain comprises a condensation domain (C domain), an adenylation domain (A domain), and a thiolation domain (T domain),
wherein the engineered polypeptide comprises a deletion of at least a portion of a C domain, a portion of an A domain, or a portion of a T domain, relative to the naturally occurring lipopeptide synthetase polypeptide, and wherein the engineered polypeptide comprises a fatty acid linkage domain.

2. The engineered polypeptide of claim 1, wherein the engineered polypeptide produces a lipopeptide having one less amino acid than a lipopeptide produced by the naturally occurring polypeptide, when expressed under conditions in which the naturally occurring polypeptide produces the naturally occurring lipopeptide.

3. The engineered polypeptide of claim 1, wherein the engineered polypeptide comprises a deletion of at least a C domain and an A domain, relative to the naturally occurring lipopeptide synthetase polypeptide.

4. The engineered polypeptide of claim 3, wherein the engineered polypeptide comprises a deletion of a C domain and A domain of the second peptide synthetase domain.

5. The engineered polypeptide of claim 4, wherein the engineered polypeptide comprises a C domain and A domain of the first peptide synthetase domain, fused to a T domain which is a hybrid T domain comprising a portion of the T domain from the first peptide synthetase domain, and a portion of the T domain from the second peptide synthetase domain.

6. The engineered polypeptide of claim 5, wherein the engineered polypeptide comprises the C and A domains of the first module of SrfA-A, fused to a T domain which comprises a portion of the T domain of the first module of SrfA-A and a portion of the T domain of the second module of SrfA-A.

7. The engineered lipopeptide synthetase polypeptide of claim 1, wherein the engineered polypeptide comprises a deletion of an A domain and a T domain, relative to the naturally occurring lipopeptide synthetase polypeptide.

8. The engineered polypeptide of claim 7, wherein the engineered polypeptide comprises an A domain and T domain of the second peptide synthetase domain, fused to a C domain which is a hybrid C domain comprising a portion of the C domain of the first peptide synthetase domain and a portion of the C domain of the second peptide synthetase domain.

9. The engineered polypeptide of claim 8, wherein the engineered polypeptide comprises a C domain which comprises a portion of the C domain of the first module of SrfA-A and a portion of the C domain of the second module of SrfA-A, fused to the A domain and T domain of the second module of SrfA-A.

10. An engineered lipopeptide synthetase polypeptide comprising:
a first peptide synthetase domain of a first peptide synthetase polypeptide, and
a second peptide synthetase domain of a second peptide synthetase polypeptide,
wherein the first and second peptide synthetase domains are covalently linked such that the engineered lipopeptide synthetase polypeptide produces a lipopeptide comprising an amino acid encoded by the first peptide synthetase domain linked to an amino acid encoded by the second peptide synthetase domain.

11. The engineered polypeptide of claim 10, wherein the first peptide synthetase domain comprises the first module of SrfA-A, and wherein the second peptide synthetase domain comprises a second module of a heterologous synthetase.

12. The engineered polypeptide of claim 10, further comprising a third peptide synthetase domain.

13. An engineered lipopeptide synthetase polypeptide comprising:
a first peptide synthetase domain of a naturally occurring lipopeptide synthetase polypeptide, and
a second peptide synthetase domain of a naturally occurring lipopeptide synthetase polypeptide, wherein the second peptide synthetase domain is covalently linked to a thioesterase domain of a peptide synthetase polypeptide.

14. The engineered polypeptide of claim 13, wherein the first peptide synthetase domain and the second peptide synthetase domain are domains from the same naturally occurring lipopeptide synthetase polypeptide.

15. The engineered polypeptide of claim 13, wherein the first and second domains are from SrfA-A.

16. The engineered polypeptide of claim 13, wherein the first peptide synthetase domain and the thioesterase domain are from the same naturally occurring lipopeptide synthetase polypeptide complex.

17. The engineered polypeptide of claim 16, wherein the first peptide synthetase domain and thioesterase domains are from the SrfA complex.

18-21. (canceled)

22. A transgenic plant comprising the engineered polypeptide of claim 1.

23. A lipopeptide produced by the engineered polypeptide of claim 1.

24. A lipopeptide consisting of the following amino acid sequence: L-Glu-D-Leu-L-Val-L-Asp-D-Leu-L-Leu (SEQ ID NO:______), wherein the lipopeptide comprises a fatty acid moiety on the L-Glu residue.

25. The lipopeptide of claim 24, wherein the lipopeptide is cyclic.

26. A lipopeptide consisting of the following amino acid sequence: L-Glu-X-D-Leu-L-Val-L-Asp-D-Leu-L-Leu (SEQ ID NO:______), wherein X is any amino acid, and wherein the lipopeptide comprises a fatty acid moiety on the L-Glu residue.

27. The lipopeptide of claim 26, wherein X is L-Tyr.

28. The lipopeptide of claim 26, wherein the lipopeptide is cyclic.

29. A lipopeptide consisting of the following amino acid sequence: L-Leu-D-Leu-L-Val-L-Asp-D-Leu-L-Leu (SEQ ID NO:______), wherein the lipopeptide comprises a fatty acid moiety on the first L-Leu residue.

30. The lipopeptide of claim 29, wherein the lipopeptide is cyclic.

31-34. (canceled)


SEQUENCE LISTING (226 sequences)

SEQ ID NO: 1 - protein, 6 aa, Artificial sequence
  Conserved sequence motif of T domain
XGGXSX

SEQ ID NO: 2 - protein, 7 aa, Artificial sequence
  Conserved sequence motif of C domain
HHXXXDG

SEQ ID NO: 3 - protein, 29 aa, Artificial sequence
  Consensus sequence for reductase domain
XXXXXXXXXXXXXYXXXKXXXXXXXXXXX

SEQ ID NO: 4 - protein, 10 aa, Artificial sequence
  Consensus sequence of thioesterase domain
XXXXGXSXGX

SEQ ID NO: 5 - DNA, 27 bp, Artificial sequence
  Synthetic primer 1C
atggtgatgctttccgcttactatacg

SEQ ID NO: 6 - DNA, 27 bp, Artificial sequence
  Synthetic primer 1D
cgttccggaagtatacgtcaggttggc

SEQ ID NO: 7 - DNA, 48 bp, Artificial sequence
  Synthetic primer TAIL-5′-1CD-FW-MOD
ctgcgatcagtgttcccactaatggtgatgctttccgcttactatacg

SEQ ID NO: 8 - DNA, 47 bp, Artificial sequence
  Synthetic primer TAIL-3′-1CD-BK-MOD
ctctggactgtcgaaagcaagcgttccggaagtatacgtcaggttgg

SEQ ID NO: 9 - DNA, 45 bp, Artificial sequence
  Synthetic primer pUC19 sense-2
cttgctttcgacagtccagaggccagtgaattcgagctcggtacc

SEQ ID NO: 10 - DNA, 49 bp, Artificial sequence
  Synthetic primer pUC19 anti-2
tagtgggaacactgatcgcagacccaacttaatcgccttgcagcacatc

SEQ ID NO: 11 - DNA, 48 bp, Artificial sequence
  Synthetic primer 1-D-cloning-BK-MOD
aacaccctttggctgacctgucgttccggaagtatacgtcaggttggc

SEQ ID NO: 12 - DNA, 48 bp, Artificial sequence
  Synthetic primer 1-DR-FW-48bp-MOD
ctaatgagtgagctaatctcucctcttgattctgcagcaatggccaac

SEQ ID NO: 13 - DNA, 48 bp, Artificial sequence; identical to SEQ ID NO: 11
  Synthetic primer 1-DR-cloning-BK-MOD

SEQ ID NO: 14 - DNA, 46 bp, Artificial sequence
  Synthetic primer 1-DR-FW-78bp-MOD
ctaatgagtgagctaatctcutatcatgccgatgcgcgaaatctcg

SEQ ID NO: 15 - DNA, 48 bp, Artificial sequence; identical to SEQ ID NO: 11
  Synthetic primer 1-DR-cloning-BK-MOD

SEQ ID NO: 16 - DNA, 44 bp, Artificial sequence
  Synthetic primer 1-DR-FW-102bp-MOD
ctaatgagtgagctaatctcugtgctagccgatgaggaagaaag

SEQ ID NO: 17 - DNA, 49 bp, Artificial sequence
  Synthetic primer PUC19-anti-3-MOD
agagattagctcactcattaggcaccccaggctttacactttatgcttc

SEQ ID NO: 18 - DNA, 47 bp, Artificial sequence
  Synthetic primer 1-pUC19-4-DR-sense-3-MOD
acaggtcagccaaagggtgtuccagctgcattaatgaatcggccaac

SEQ ID NO: 19 - DNA, 31 bp, Artificial sequence
  Synthetic primer 1-G
gacctctgttgtattgaatgacgtcttcctg

SEQ ID NO: 20 - DNA, 31 bp, Artificial sequence
  Synthetic primer 1-H
accggacagccgaagggtgtcatggtcgagc

SEQ ID NO: 21 - DNA, 48 bp, Artificial sequence
  Synthetic primer 1-DR-HG-FW
atggtcgagcatcatgcgctugtgaacctttgcttctggcaccacgac

SEQ ID NO: 22 - DNA, 50 bp, Artificial sequence
  Synthetic primer 1-HG-4-vect+DR-BK
gagtgcagaatactcaaaccggacctctgttgtattgaatgacgtcttcc

SEQ ID NO: 23 - DNA, 46 bp, Artificial sequence
  Synthetic primer 1-DR-in-Vect-4-HG-BK
aagcgcatgatgctcgaccauaacaccctttggctgacctgtcgtt

SEQ ID NO: 24 - DNA, 45 bp, Artificial sequence
  Synthetic primer Vector-FW-4-HG-1&2
cggtttgagtattctgcactcttccgcttcctcgctcactgactc

SEQ ID NO: 25 - DNA, 32 bp, Artificial sequence
  Synthetic primer 1CD-and-2CD-FW
ggatgtgctgcaaggcgattaagttgggtctg

SEQ ID NO: 26 - DNA, 47 bp, Artificial sequence
  Synthetic primer 1CD-BstXI-BK
atgctaatccactctcttggcgttccggaagtatacgtcaggttggc

SEQ ID NO: 27 - DNA, 50 bp, Artificial sequence
  Synthetic primer UPP-KAN-BstXI-FW
atgctaagccaagagagtgggttttttgacgatgttcttgaaactcaatg

SEQ ID NO: 28 - DNA, 52 bp, Artificial sequence
  Synthetic primer UPP-KAN-and-KAN-BglI-BK
atatctgagccagagaggcaccaatcaaaaaacagatggccgctattaaagc

SEQ ID NO: 29 - DNA, 49 bp, Artificial sequence
  Synthetic primer 1HG-and-2HG-BglI-FW
attactacgcctctctggcacacaacatacgagccggaagcataaagtg

SEQ ID NO: 30 - DNA, 36 bp, Artificial sequence
  Synthetic primer 1HG-and-2HG-BK
tgattctgtggataaccgtattaccgcctttgagtg

SEQ ID NO: 31 - protein, 26 aa, Artificial sequence
  Synthetic module
WQDVLNVEKAGIFDNFFETGGHSLKA

SEQ ID NO: 32 - protein, 28 aa, Artificial sequence
  Synthetic module
WAQVLQAEQVGAYDHFFDIGGHSLAGMK

SEQ ID NO: 33 - protein, 27 aa, Artificial Sequence
  Synthetic module
WQDVLGMSEVGVTDNFFSLGGDSIKGI

SEQ ID NO: 34 - protein, 28 aa, Artificial sequence
  Synthetic reference module
WAEVLGVDPDEIGIDDNFFELGGDAVLE

SEQ ID NO: 35 - DNA, 30 bp, Artificial sequence
  Synthetic primer 2C-MOD
catcatttggttgaatctctgcagcagacg

SEQ ID NO: 36 - DNA, 25 bp, Artificial sequence
  Synthetic primer 2D
gtcaaagatccccgccttctcaacg

SEQ ID NO: 37 - DNA, 50 bp, Artificial sequence
  Synthetic primer TAIL-5′-2CD-FW-MOD
ctgcgatcagtgttcccactacatcatttggttgaatctctgcagcagac

SEQ ID NO: 38 - DNA, 46 bp, Artificial sequence
  Synthetic primer TAIL-3′-2CD-BK-MOD
ctctggactgtcgaaagcaaggtcaaagatccccgccttctcaacg

SEQ ID NO: 39 - DNA, 45 bp, Artificial sequence; identical to SEQ ID NO: 9
  pUC19 sense-2

SEQ ID NO: 40 - DNA, 49 bp, Artificial sequence; identical to SEQ ID NO: 10
  Synthetic primer pUC19 anti-2

SEQ ID NO: 41 - DNA, 46 bp, Artificial sequence
  Synthetic primer 2-DR-cloning-BK-MOD
tcctccaatgtcaaagaagtggtcaaagatccccgccttctcaacg

SEQ ID NO: 42 - DNA, 48 bp, Artificial sequence
  Synthetic primer 2-DR-FW-45bp-MOD
ctaatgagtgagctaatctcuatttggcaggacgtgctgaacgttgag

SEQ ID NO: 43 - DNA, 46 bp, Artificial sequence; identical to SEQ ID NO: 41
  Synthetic primer 2-DR-cloning-BK-MOD

SEQ ID NO: 44 - DNA, 49 bp, Artificial sequence
  Synthetic primer 2-DR-FW-78bp-MOD
ctaatgagtgagctaatctcuccgcgaaatgagactgaaaaagcaatcg

SEQ ID NO: 45 - DNA, 46 bp, Artificial sequence; identical to SEQ ID NO: 41
  Synthetic primer 2-DR-cloning-BK-MOD

SEQ ID NO: 46 - DNA, 45 bp, Artificial sequence
  Synthetic primer 2-DR-FW-102bp-MOD
ctaatgagtgagctaatctcugtcagcggcactgcctatacagcg

SEQ ID NO: 47 - DNA, 49 bp, Artificial sequence; identical to SEQ ID NO: 17
  Synthetic primer PUC19-anti-3-MOD

SEQ ID NO: 48 - DNA, 47 bp, Artificial sequence
  Synthetic primer 2-pUC19-4-DR-sense-3-MOD
cacttctttgacattggaggaccagctgcattaatgaatcggccaac

SEQ ID NO: 49 - DNA, 33 bp, Artificial sequence
  Synethic primer 2-G
gtcacgctgaacctgaacatttccgatcaaatc

SEQ ID NO: 50 - DNA, 32 bp, Artificial sequence
  Synthetic primer 2-H
cacttctttgacattggcggacattcattagc

SEQ ID NO: 51 - DNA, 49 bp, Artificial sequence
  Synthetic primer 2-DR-HG-FW
cattctttagctggtatgaagatgcctgccttggttcatcaagaactgg

SEQ ID NO: 52 - DNA, 49 bp, Artificial sequence
  Synthetic primer 2-HG-4-vect+DR-BK
gagtgcagaatactcaaaccggtcacgctgaacctgaacatttccgatc

SEQ ID NO: 53 - DNA, 49 bp, Artificial sequence
  Synthetic primer 2-DR-in-Vect-4-HG-BK#1
cttcataccagctaaagaatgtcctccaatgtcaaagaagtggtcaaag

SEQ ID NO: 54 - DNA, 45 bp, Artificial sequence; identical to SEQ ID NO: 24
  Synthetic primer Vector-FW-4-HG-1&2

SEQ ID NO: 55 - DNA, 32 bp, Artificial sequence; identical to SEQ ID NO: 25
  Synthetic primer 1CD-and-2CD-FW

SEQ ID NO: 56 - DNA, 45 bp, Artificial sequence
  Synthetic primer 2CD-BstXI-BK
atgctaatccactctcttgggtcaaagataccagccttctcaacg

SEQ ID NO: 57 - DNA, 50 bp, Artificial sequence; identical to SEQ ID NO: 27
  Synthetic primer UPP-KAN-BstXI-FW

SEQ ID NO: 58 - DNA, 52 bp, Artificial sequence; identical to SEQ ID NO: 28
  Synthetic primer UPP-KAN-and-KAN-BglI-BK

SEQ ID NO: 59 - DNA, 48 bp, Artificial sequence
  Synthetic primer 1HG-and-2HG-BglI-FW
attactacgcctctctggcacacaacatacgagccggaagcataaagt

SEQ ID NO: 60 - DNA, 36 bp, Artificial sequence; identical to SEQ ID NO: 30
  Synthetic primer 1HG-and-2HG-BK

SEQ ID NO: 61 - protein, 10 aa, Artificial sequence
  Synthetic
LNVEKAGIFD

SEQ ID NO: 62 - protein, 10 aa, Artificial sequence
  Synthetic
HFFELGGHSL

SEQ ID NO: 63 - protein, 10 aa, Artificial sequence
  Synthetic
LGVSGIGILD

SEQ ID NO: 64 - protein, 10 aa, Artificial sequence
  Synthetic
HFFDIGGHSL

SEQ ID NO: 65 - protein, 10 aa, Artificial sequence
  Synthetic
LNVEKAGIFD

SEQ ID NO: 66 - protein, 10 aa, Artificial sequence
  Synthetic
NFFELGGHSL

SEQ ID NO: 67 - protein, 10 aa, Artificial sequence
  Synthetic
LGVETIGVHD

SEQ ID NO: 68 - protein, 10 aa, Artificial sequence
  Synthetic
HFFDIGGHSL

SEQ ID NO: 69 - protein, 10 aa, Artificial sequence
  Synthetic
LNVEKAGIFD

SEQ ID NO: 70 - protein, 10 aa, Artificial sequence
  Synthetic
HFFTLGGHSL

SEQ ID NO: 71 - protein, 10 aa, Artificial sequence
  Synthetic
LGISGVGVLD

SEQ ID NO: 72 - protein, 10 aa, Artificial sequence
  Synthetic
HFFDIGGHSL

SEQ ID NO: 73 - protein, 20 aa, Artificial sequence
  Synthetic
KAIAAIWQDVLNVEKAGIFD

SEQ ID NO: 74 - protein, 20 aa, Artificial sequence
  Synthetic
HFFDIGGHSLAGMKMLALVH

SEQ ID NO: 75 - protein, 20 aa, Artificial sequence
  Synthetic
KAIAAIWQDVLNVEKAGIFD

SEQ ID NO: 76 - protein, 19 aa, Artificial sequence
  Synthetic
HFDIGGHSLAGMKMPALVH

SEQ ID NO: 77 - protein, 25 aa, Artificial sequence
  Synthetic
PEADAELIDLDQAIEEGAEESLNAD

SEQ ID NO: 78 - protein, 28 aa, Artificial sequence
  Synthetic
ADEEESYHADARNLALPLDSAAMANLTY

SEQ ID NO: 79 - protein, 25 aa, Artificial sequence
  Synthetic
PEADAELIDLDQAIEEGAEESLNAD

SEQ ID NO: 80 - protein, 26 aa, Artificial sequence
  Synthetic
EEESYHADARNLALPLDSAAMANLTY

SEQ ID NO: 81 - protein, 24 aa, Artificial sequence
  Synthetic
PEADAELIDLDQAIEEGAEESLNA

SEQ ID NO: 82 - protein, 20 aa, Artificial sequence
  Synthetic
ADARNLALPLDSAAMANLTY

SEQ ID NO: 83 - protein, 24 aa, Artificial sequence
  Synthetic
PEADAELIDLDQAIEEGAEESLNA

SEQ ID NO: 84 - protein, 17 aa, Artificial sequence
  Synthetic
RNLALPLDSAAMANLTY

SEQ ID NO: 85 - protein, 23 aa, Artificial sequence
  Synthetic
PEADAELIDLDQAIEEGAEESLN

SEQ ID NO: 86 - protein, 27 aa, Artificial sequence
  Synthetic
DEEESYHADARNLALPLDSAAMANLTY

SEQ ID NO: 87 - protein, 23 aa, Artificial sequence
  Synthetic
PEADAELIDLDQAIEEGAEESLN

SEQ ID NO: 88 - protein, 25 aa, Artificial sequence
  Synthetic
EESYHADARNLALPLDSAAMANLTY

SEQ ID NO: 89 - protein, 23 aa, Artificial sequence
  Synthetic
PEADAELIDLDQAIEEGAEESLN

SEQ ID NO: 90 - protein, 23 aa, Artificial sequence
  Synthetic
SYHADARNLALPLDSAAMANLTY

SEQ ID NO: 91 - protein, 23 aa, Artificial sequence
  Synthetic
PEADAELIDLDQAIEEGAEESLN

SEQ ID NO: 92 - protein, 21 aa, Artificial sequence
  Synthetic
HADARNLALPLDSAAMANLTY

SEQ ID NO: 93 - protein, 23 aa, Artificial sequence
  Synthetic
PEADAELIDLDQAIEEGAEESLN

SEQ ID NO: 94 - protein, 20 aa, Artificial sequence
  Synthetic
ADARNLALPLDSAAMANLTY

SEQ ID NO: 95 - protein, 23 aa, Artificial sequence
  Synthetic
PEADAELIDLDQAIEEGAEESLN

SEQ ID NO: 96 - protein, 17 aa, Artificial sequence
  Synthetic
RNLALPLDSAAMANLTY

SEQ ID NO: 97 - protein, 23 aa, Artificial sequence
  Synthetic
PEADAELIDLDQAIEEGAEESLN

SEQ ID NO: 98 - protein, 14 aa, Artificial sequence
  Synthetic
ALPLDSAAMANLTY

SEQ ID NO: 99 - protein, 21 aa, Artificial sequence
  Synthetic
PEADAELIDLDQAIEEGAEES

SEQ ID NO: 100 - protein, 27 aa, Artificial sequence
  Synthetic
DEEESYHADARNLALPLDSAAMANLTY

SEQ ID NO: 101 - protein, 21 aa, Artificial sequence
  Synthetic
PEADAELIDLDQAIEEGAEES

SEQ ID NO: 102 - protein, 25 aa, Artificial sequence
  Synthetic
EESYHADARNLALPLDSAAMANLTY

SEQ ID NO: 103 - protein, 21 aa, Artificial sequence
  Synthetic
PEADAELIDLDQAIEEGAEES

SEQ ID NO: 104 - protein, 23 aa, Artificial sequence
  Synthetic
SYHADARNLALPLDSAAMANLTY

SEQ ID NO: 105 - protein, 21 aa, Artificial sequence
  Synthetic
PEADAELIDLDQAIEEGAEES

SEQ ID NO: 106 - protein, 22 aa, Artificial sequence
  Synthetic
YHADARNLALPLDSAAMANLTY

SEQ ID NO: 107 - protein, 21 aa, Artificial sequence
  Synthetic
PEADAELIDLDQAIEEGAEES

SEQ ID NO: 108 - protein, 17 aa, Artificial sequence
  Synthetic
RNLALPLDSAAMANLTY

SEQ ID NO: 109 - protein, 21 aa, Artificial sequence
  Synthetic
PEADAELIDLDQAIEEGAEES

SEQ ID NO: 110 - protein, 15 aa, Artificial sequence
  Synthetic
LALPLDSAAMANLTY

SEQ ID NO: 111 - protein, 20 aa, Artificial sequence
  Synthetic
PEADAELIDLDQAIEEGAEE

SEQ ID NO: 112 - protein, 25 aa, Artificial sequence
  Synthetic
EESYHADARNLALPLDSAAMANLTY

SEQ ID NO: 113 - protein, 20 aa, Artificial sequence
  Synthetic
PEADAELIDLDQAIEEGAEE

SEQ ID NO: 114 - protein, 23 aa, Artificial sequence
  Synthetic
SYHADARNLALPLDSAAMANLTY

SEQ ID NO: 115 - protein, 20 aa, Artificial sequence
  Synthetic
PEADAELIDLDQAIEEGAEE

SEQ ID NO: 116 - protein, 21 aa, Artificial sequence
  Synthetic
HADARNLALPLDSAAMANLTY

SEQ ID NO: 117 - protein, 20 aa, Artificial sequence
  Synthetic
PEADAELIDLDQAIEEGAEE

SEQ ID NO: 118 - protein, 17 aa, Artificial sequence
  Synthetic
RNLALPLDSAAMANLTY

SEQ ID NO: 119 - protein, 20 aa, Artificial sequence
  Synthetic
PEADAELIDLDQAIEEGAEE

SEQ ID NO: 120 - protein, 15 aa, Artificial sequence
  Synthetic
LALPLDSAAMANLTY

SEQ ID NO: 121 - protein, 17 aa, Artificial sequence
  Synthetic
PEADAELIDLDQAIEEG

SEQ ID NO: 122 - protein, 27 aa, Artificial sequence
  Synthetic
DEEESYHADARNLALPLDSAAMANLTY

SEQ ID NO: 123 - protein, 17 aa, Artificial sequenc
  Synthetic
PEADAELIDLDQAIEEG

SEQ ID NO: 124 - protein, 25 aa, Artificial sequence
  Synthetic
EESYHADARNLALPLDSAAMANLTY

SEQ ID NO: 125 - protein, 17 aa, Artificial sequence
  Synthetic
PEADAELIDLDQAIEEG

SEQ ID NO: 126 - protein, 23 aa, Artificial sequence
  Synthetic
SYHADARNLALPLDSAAMANLTY

SEQ ID NO: 127 - protein, 17 aa, Artificial sequence
  Synthetic
PEADAELIDLDQAIEEG

SEQ ID NO: 128 - protein, 21 aa, Artificial sequence
  Synthetic
HADARNLALPLDSAAMANLTY

SEQ ID NO: 129 - protein, 17 aa, Artificial sequence
  Synthetic
PEADAELIDLDQAIEEG

SEQ ID NO: 130 - protein, 20 aa, Artificial sequence
  Synthetic
ADARNLALPLDSAAMANLTY

SEQ ID NO: 131 - protein, 17 aa, Artificial sequence
  Synthetic
PEADAELIDLDQAIEEG

SEQ ID NO: 132 - protein, 17 aa, Artificial sequence
  Synthetic
RNLALPLDSAAMANLTY

SEQ ID NO: 133 - protein, 26 aa, Artificial sequence
  Synthetic
ADEEESYHADARNLALPLDSAAMANL

SEQ ID NO: 134 - protein, 8 aa, Artificial sequence
  Synthetic
EENPENPE

SEQ ID NO: 135 - protein, 25 aa, Artificial sequence
  Synthetic
ADEEESYHADARNLALPLDSAAMAN

SEQ ID NO: 136 - protein, 20 aa, Artificial sequence
  Synthetic
RTILSLPLDENDEENPENPE

SEQ ID NO: 137 - protein, 25 aa, Artificial sequence
  Synthetic
ADEEESYHADARNLALPLDSAAMAN

SEQ ID NO: 138 - protein, 14 aa, Artificial sequence
  Synthetic
PLDENDEENPENPE

SEQ ID NO: 139 - protein, 25 aa, Artificial sequence
  Synthetic
ADEEESYHADARNLALPLDSAAMAN

SEQ ID NO: 140 - protein, 16 aa, Artificial sequence
  Synthetic
SLPLDENDEENPENPE

SEQ ID NO: 141 - protein, 25 aa, Artificial sequence
  Synthetic
ADEEESYHADARNLALPLDSAAMAN

SEQ ID NO: 142 - protein, 4 aa, Artificial sequence
  Synthetic
ENPE

SEQ ID NO: 143 - protein, 13 aa, Artificial sequence
  Synthetic
ADEEESYHADARN

SEQ ID NO: 144 - protein, 11 aa, Artificial sequence
  Synthetic
ADEEESYHADA

SEQ ID NO: 145 - protein, 20 aa, Artificial sequence
  Synthetic
RTILSLPLDENDEENPENPE

SEQ ID NO: 146 - protein, 26 aa, Artificial sequence
  Synthetic
ADEEESYHADARNLALPLDSAAMANL

SEQ ID NO: 147 - protein, 6 aa, Artificial sequence
  Synthetic
NPENPE

SEQ ID NO: 148 - protein, 26 aa, Artificial sequence
  Synthetic
ADEEESYHADARNLALPLDSAAMANL

SEQ ID NO: 149 - protein, 12 aa, Artificial sequence
  Synthetic
DENDEENPENPE

SEQ ID NO: 150 - protein, 11 aa, Artificial sequence
  Synthetic
ADEEESYHADA

SEQ ID NO: 151 - protein, 13 aa, Artificial sequence
  Synthetic
ADEEESYHADARN

SEQ ID NO: 152 - protein, 14 aa, Artificial sequence
  Synthetic
PLDENDEENPENPE

SEQ ID NO: 153 - DNA, 22 bp, Artificial sequence
  Synthetic primer
tattgtcgggaatgcgatcatg

SEQ ID NO: 154 - DNA, 24 bp, Artificial sequence
  Synthetic primer
agattcaaccaaatgatgaacctg

SEQ ID NO: 155 - DNA, 22 bp, Artificial sequence
  Synthetic primer
tattgtcgggaatgcgatcatg

SEQ ID NO: 156 - DNA, 47 bp, Artificial sequence
  Synthetic primer
atgtgctaccactcctctggatcagcattcaggctttcttctgcacc

SEQ ID NO: 157 - DNA, 50 bp, Artificial sequence
  Synthetic primer
atgctaagccagaggagtgggttttttgacgatgttcttgaaactcaatg

SEQ ID NO: 158 - DNA, 51 bp, Artificial sequence
  Synthetic primer
atgtgctaccaacttcctggcagagtatggacagttgcggatgtacttcag

SEQ ID NO: 159 - DNA, 24 bp, Artificial sequence
  Synthetic primer
atgcagcatttcttccgtgacagc

SEQ ID NO: 160 - DNA, 26 bp, Artificial sequence
  Synthetic primer
gcagctcgtccatttggataaacacc

SEQ ID NO: 161 - DNA, 46 bp, Artificial sequence
  Synthetic primer
atatctgtccaggaagttgggccgatgaggaagaaagctatcatgc

SEQ ID NO: 162 - DNA, 26 bp, Artificial sequence
  Synthetic primer
gcagctcgtccatttggataaacacc

SEQ ID NO: 163 - DNA, 50 bp, Artificial sequence
  Synthetic primer
aaacaatttgaatctgtgccugaacttgtctctttgaaacggaatgcatc

SEQ ID NO: 164 - DNA, 47 bp, Artificial sequence
  Synthetic primer
atagctttcttcctcatcggcatcagcattcaggctttcttctgcac

SEQ ID NO: 165 - DNA, 45 bp, Artificial sequence
  Synthetic primer
gccgatgaggaagaaagctauaccagtgaattagagctcggtacc

SEQ ID NO: 166 - DNA, 49 bp, Artificial sequence
  Synthetic primer
aggcacagattcaaattgttuacccaacttaatcgccttgcagcacatc

SEQ ID NO: 167 - DNA, 50 bp, Artificial sequence
  Synthetic
gccgatgaggaagaaagctaucatgcagacgcaagaaatctcgcactgcc

SEQ ID NO: 168 - DNA, 48 bp, Artificial sequence
  Synthetic
aatttttccattccctgtcagcggcagctcgtccatttggataaacac

SEQ ID NO: 169 - DNA, 45 bp, Artificial sequence
  Synthetic
ctgacagggaatggaaaaatuttacgcttactcgctcactgactc

SEQ ID NO: 170 - DNA, 47 bp, Artificial sequence; identical to SEQ ID NO: 164
  Synthetic

SEQ ID NO: 171 - DNA, 47 bp, Artificial sequence
  Synthetic
gaatgctgatgaggaagaaagctatcatgcagacgcaagaaatctcg

SEQ ID NO: 172 - DNA, 45 bp, Artificial sequence
  Synthetic primer
ctttcttcctcatcagcattcaggctttcttctgcaccttcctca

SEQ ID NO: 173 - DNA, 45 bp, Artificial sequence
  Synthetic primer
aagaaagcctgaatgctgcagacgcaagaaatctcgcactgcctc

SEQ ID NO: 174 - DNA, 45 bp, Artificial sequence
  Synthetic primer
ctgcagcattcaggctttctuctgcaccttcctcaatcgcctgat

SEQ ID NO: 175 - DNA, 45 bp, Artificial sequence
  Synthetic primer
aagcctgaatgctagaaatcucgcactgcctcttgattctgcagc

SEQ ID NO: 176 - DNA, 45 bp, Artificial sequence
  Synthetic primer
agatttctagcattcaggctutcttctgcaccttcctcaatcgcc

SEQ ID NO: 177 - DNA, 45 bp, Artificial sequence
  Synthetic
aagaaagcctgaatgctctcgcactgcctcttgattctgcagcaa

SEQ ID NO: 178 - DNA, 44 bp, Artificial sequence
  Synthetic primer
cgagagcattcaggctttctuctgcaccttcctcaatcgcctga

SEQ ID NO: 179 - DNA, 46 bp, Artificial sequence
  Synthetic primer
gaatgatgaggaagaaagctaucatgcagacgcaagaaatctcgca

SEQ ID NO: 180 - DNA, 46 bp, Artificial sequence
  Synthetic primer
atagctttcttcctcatcattcaggctttcttctgcaccttcctca

SEQ ID NO: 181 - DNA, 45 bp, Artificial sequence
  Synthetic primer
gaatgaagaaagctatcatgcagacgcaagaaatctcgcactgcc

SEQ ID NO: 182 - DNA, 45 bp, Artificial sequence
  Synthetic primer
gcatgatagctttcttcattcaggctttcttctgcaccttcctca

SEQ ID NO: 183 - DNA, 45 bp, Artificial sequence
  Synthetic primer
aaagcctgaatagctatcatgcagacgcaagaaatctcgcactgc

SEQ ID NO: 184 - DNA, 45 bp, Artificial sequence
  Synthetic primer
catgatagctattcaggcttucttctgcaccttcctcaatcgcct

SEQ ID NO: 185 - DNA, 45 bp, Artificial sequence
  Synthetic primer
cagaagaaagcctgaatcatgcagacgcaagaaatctcgcactgc

SEQ ID NO: 186 - DNA, 46 bp, Artificial sequence
  Synthetic primer
catgattcaggctttcttctgcaccttcctcaatcgcctgatctaa

SEQ ID NO: 187 - DNA, 45 bp, Artificial sequence
  Synthetic primer
gaagaaagcctgaatgcagacgcaagaaatctcgcactgcctctt

SEQ ID NO: 188 - DNA, 45 bp, Artificial sequence
  Synthetic primer
gtctgcattcaggctttcttctgcaccttcctcaatcgcctgatc

SEQ ID NO: 189 - DNA, 45 bp, Artificial sequence
  Synthetic primer
agaagaaagcctgaatagaaauctcgcactgcctcttgattctgc

SEQ ID NO: 190 - DNA, 47 bp, Artificial sequence
  Synthetic primer
atttctattcaggctttcttcugcaccttcctcaatcgcctgatcta

SEQ ID NO: 191 - DNA, 45 bp, Artificial sequence
  Synthetic primer
cagaagaaagcctgaatctcgcactgcctcttgattctgcagcaa

SEQ ID NO: 192 - DNA, 45 bp, Artificial sequence
  Synthetic primer
cgagattcaggctttcttctgcaccttcctcaatcgcctgatcta

SEQ ID NO: 193 - DNA, 47 bp, Artificial sequence
  Synthetic primer
agaaagcgatgaggaagaaagctatcatgcagacgcaagaaatctcg

SEQ ID NO: 194 - DNA, 45 bp, Artificial sequence
  Synthetic primer
ctttcttcctcatcgctttcutctgcaccttcctcaatcgcctga

SEQ ID NO: 195 - DNA, 45 bp, Artificial sequence
  Synthetic primer
gaagaaagcgaagaaagctaucatgcagacgcaagaaatctcgca

SEQ ID NO: 196 - DNA, 45 bp, Artificial sequence
  Synthetic primer
atagctttcttcgctttcttctgcaccttcctcaatcgcctgatc

SEQ ID NO: 197 - DNA, 45 bp, Artificial sequence
  Synthetic primer
cagaagaaagcagctatcatgcagacgcaagaaatctcgcactgc

SEQ ID NO: 198 - DNA, 46 bp, Artificial sequence
  Synthetic primer
catgatagctgctttcttctgcaccttcctcaatcgcctgatctaa

SEQ ID NO: 199 - DNA, 45 bp, Artificial sequence
  Synthetic primer
gcagaagaaagcagaaatctcgcactgcctcttgattctgcagca

SEQ ID NO: 200 - DNA, 47 bp, Artificial sequence
  Synthetic primer
gagatttctgctttcttctgcaccttcctcaatcgcctgatctaagt

SEQ ID NO: 201 - DNA, 44 bp, Artificial sequence
  Synthetic primer
aaggtgcagaagaaagcctcgcactgcctcttgattctgcagca

SEQ ID NO: 202 - DNA, 48 bp, Artificial sequence
  Synthetic primer
cgaggctttcttctgcacctucctcaatcgcctgatctaagtcaatca

SEQ ID NO: 203 - DNA, 45 bp, Artificial sequence
  Synthetic primer
aagaagatgaggaagaaagcuatcatgcagacgcaagaaatctcg

SEQ ID NO: 204 - DNA, 45 bp, Artificial sequence
  Synthetic primer
agctttcttcctcatcttctuctgcaccttcctcaatcgcctgat

SEQ ID NO: 205 - DNA, 47 bp, Artificial sequence
  Synthetic primer
cagaagaagaagaaagctatcaugcagacgcaagaaatctcgcactg

SEQ ID NO: 206 - DNA, 48 bp, Artificial sequence
  Synthetic primer
atgatagctttcttcttcttctgcaccttcctcaatcgcctgatctaa

SEQ ID NO: 207 - DNA, 45 bp, Artificial sequence
  Synthetic primer
gaagaaagctatcatgcagacgcaagaaatctcgcactgcctctt

SEQ ID NO: 208 - DNA, 45 bp, Artificial sequence
  Synthetic primer
gtctgcatgatagctttcttctgcaccttcctcaatcgcctgatc

SEQ ID NO: 209 - DNA, 45 bp, Artificial sequence
  Synthetic primer
aggaaggtgcagaagaacatgcagacgcaagaaatctcgcactgc

SEQ ID NO: 210 - DNA, 47 bp, Artificial sequence
  Synthetic primer
catgttcttctgcaccttccucaatcgcctgatctaagtcaatcagc

SEQ ID NO: 211 - DNA, 45 bp, Artificial sequence
  Synthetic primer
aggtgcagaagaaagaaatcucgcactgcctcttgattctgcagc

SEQ ID NO: 212 - DNA, 48 bp, Artificial sequence
  Synthetic primer
agatttctttcttctgcaccutcctcaatcgcctgatctaagtcaatc

SEQ ID NO: 213 - DNA, 45 bp, Artificial sequence
  Synthetic primer
agaagaactcgcactgcctcutgattctgcagcaatggccaacct

SEQ ID NO: 214 - DNA, 46 bp, Artificial sequence
  Synthetic primer
agaggcagtgcgagttcttcugcaccttcctcaatcgcctgatcta

SEQ ID NO: 215 - DNA, 45 bp, Artificial sequence
  Synthetic primer
aggtgatgaggaagaaagctatcatgcagacgcaagaaatctcgc

SEQ ID NO: 216 - DNA, 48 bp, Artificial sequence
  Synthetic primer
tagctttcttcctcatcaccutcctcaatcgcctgatctaagtcaatc

SEQ ID NO: 217 - DNA, 45 bp, Artificial sequence
  Synthetic primer
gaggaaggtgaagaaagctaucatgcagacgcaagaaatctcgca

SEQ ID NO: 218 - DNA, 48 bp, Artificial sequence
  Synthetic primer
atagctttcttcaccttcctcaatcgcctgatctaagtcaatcagctc

SEQ ID NO: 219 - DNA, 45 bp, Artificial sequence
  Synthetic primer
gaaggtagctatcatgcagacgcaagaaatctcgcactgcctctt

SEQ ID NO: 220 - DNA, 48 bp, Artificial sequence
  Synthetic primer
gtctgcatgatagctaccttcctcaatcgcctgatctaagtcaatcag

SEQ ID NO: 221 - DNA, 45 bp, Artificial sequence
  Synthetic primer
attgaggaaggtcatgcagacgcaagaaatctcgcactgcctctt

SEQ ID NO: 222 - DNA, 46 bp, Artificial sequence
  Synthetic primer
gtctgcatgaccttcctcaaucgcctgatctaagtcaatcagctca

SEQ ID NO: 223 - DNA, 45 bp, Artificial sequence
  Synthetic primer
aggtgcagacgcaagaaatcucgcactgcctcttgattctgcagc

SEQ ID NO: 224 - DNA, 46 bp, Artificial sequence
  Synthetic primer
agatttcttgcgtctgcaccutcctcaatcgcctgatctaagtcaa

SEQ ID NO: 225 - DNA, 46 bp, Artificial sequence
  Synthetic primer
gattgaggaaggtagaaatctcgcactgcctcttgattctgcagca

SEQ ID NO: 226 - DNA, 48 bp, Artificial sequence
  Synthetic primer
gagatttctaccttcctcaatcgcctgatctaagtcaatcagctcagc